(19) United States
(12) Patent Application Publication
(10) Pub. No.: US 2025/0051332 A1
(43) Pub. Date: Feb. 13, 2025

(54) OPIOID RECEPTOR AGONIST AND METHODS OF USE THEREOF

(71) Applicants: THE BOARD OF TRUSTEES OF THE UNIVERSITY OF ILLINOIS, URBANA, IL (US); PURDUE RESEARCH FOUNDATION, WEST LAFAYETTE, IN (US)

(72) Inventors: ANDREW RILEY, CHICAGO, IL (US); RICHARD VAN RIJN, WEST LAFAYETTE, IN (US); ANNA GUTRIDGE, WEST LAFAYETTE, IN (US); SIMONE CREED, CHICAGO, IL (US)

(21) Appl. No.: 18/922,525

(22) Filed: Oct. 22, 2024

Related U.S. Application Data
(63) Continuation of application No. PCT/US23/66050, filed on Apr. 21, 2023.
(60) Provisional application No. 63/333,633, filed on Apr. 22, 2022.

(51) Int. Cl. C07D 471/20; A61K 31/438; A61K 31/4545; A61K 31/5377; A61P 25/04

(52) U.S. Cl. CPC C07D 471/20; A61K 31/438; A61K 31/4545; A61K 31/5377; A61P 25/04

(57) ABSTRACT

In accordance with the purpose(s) of the present disclosure, as embodied and broadly described herein, the disclosure, in one aspect, relates to compounds that can bind to one or more opioid receptors. In one aspect, the compounds described herein can bind to the kappa opioid receptor (κOR) and behave as an opioid receptor agonist. The ability of the compounds to bind to opioid receptors make them effective in the treatment or prevention of pain such as chronic pain.

### CROSS-REFERENCE TO RELATED APPLICATIONS

This application is a continuation-in-part of international application number PCT/US2023/066050, filed Apr. 21, 2023, which claims the benefit of and priority to U.S. provisional application No. 63/333,633, filed on Apr. 22, 2022, the contents of which are incorporated by reference herein in their entireties.

### BACKGROUND

Emerging evidence indicates that biased kappa opioid receptor agonists are effective in animal models of pain and itch, but lack the negative side-effects typically associated with traditional kappa opioid receptor agonists, most notably sedation and dysphoria. Furthermore, kappa opioid receptor agonists have been shown to decrease self-administration in animals trained to self-administer certain drugs of abuse (cocaine, morphine, alcohol, and nicotine). Emerging evidence indicates that the kappa opioid receptor may play a role in multiple sclerosis (MS) and agonists have shown to increases remyleination of neurons and alleviates the symptoms in animal models of MS.

Opioid analgesics such as morphine and its semisynthetic derivatives have been integral components of effective pain management. However, chronic pain remains an escalating and poorly managed health concern (˜20% of adults globally) and opioid prescriptions for the treatment of chronic pain have risen dramatically despite their reduced effectiveness against chronic pain states. The prolonged use of opioid analgesics elicits numerous adverse effects including respiratory depression, tolerance, and drug dependence. Increased prescription and duration of use of short and long-acting/extended-release opioids, combined with the side effect profile of opioid medications, has led to the current opioid epidemic characterized by >40,000 opioid overdose deaths per year since 2016. Thus, there is a need for other suitable options for managing pain without the use of opioid analgesics.

### SUMMARY

In accordance with the purpose(s) of the present disclosure, as embodied and broadly described herein, the disclosure, in one aspect, relates to compounds that can bind to one or more opioid receptors. In one aspect, the compounds described herein can bind to the kappa opioid receptor (κOR) and behave as an opioid receptor agonist. The ability of the compounds to bind to opioid receptors make them effective in the treatment or prevention of pain such as chronic pain.

In one aspect, the compounds have the formula I

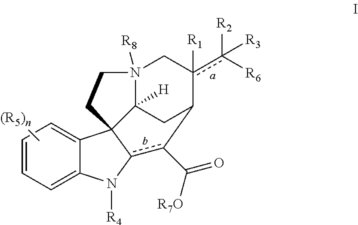

or a pharmaceutically acceptable salt thereof,
wherein
each [symbol] independently represents a single or double bond;
R₁ is absent when [symbol] is a double bond or R₁ is hydrogen, hydroxyl, or acetyloxy when [symbol] is a single bond;
R₂ is hydrogen;
R₃ is absent when [symbol] is a double bond or is hydrogen, hydroxyl, or acetyloxy when [symbol] is a single bond, or R₂ and R₃ together form a carbonyl group when [symbol] is a single bond;
R₄ is hydrogen, substituted or unsubstituted alkyl, or aralkyl;
R₆ and R₇ are alkyl; and
R₅ is selected from hydrogen, hydroxy, alkoxy, hydroxyalkyl, alkoxyalkyl, hale, haloalkyl, nitrile, nitro, amino, amido, acetyl, acetylamino, acetyloxy, substituted or unsubstituted alkyl, substituted or unsubstituted cycloalkyl, substituted or unsubstituted heterocycloalkyl, substituted or unsubstituted aryl, or substituted or unsubstituted heteroaryl;
n is 1, 2, 3, or 4; and
R₈ is absent or is a substituted or unsubstituted alkyl, an alkenyl group, or an aralkyl group.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows exemplary procedures for modifying the C10 position of akuammicine.

FIG. 2 shows exemplary procedures for the oxidation and reduction of akuammicine olefins.

FIG. 3 shows exemplary procedures for the synthesis of quaternary amines of akuammicine.

FIG. 4 shows exemplary procedures for the synthesis of doubly modified derivatives.

FIGS. 5A-5D show the pharmacological characterization of the akuammicine (1) analogues 2-6 at the μOR and κOR. Compounds 1-6 were evaluated using the HitHunter assay that measures inhibition of forskolin-induced cAMP at the κOR (A and C) μOR (B and D). All curves are representative of the averaged values from a minimum of three independent assays.

FIG. 6 shows β-arrestin-2 (βArr2) recruitment to the κOR induced by 1, 2, 3, and 5. Recruitment of @Arr2 in a PathHunter assay at κOR of 1-3 and 5 with U50,488 as a control. All curves are representative of the averaged values from a minimum of three independent assays.

FIG. 7 shows GPCRome screening of compounds 1, 2, and 3 using the Presto-Tango Assay. Data collected by the PDSP using PRESTO-Tango to measure @Arr recruitment at 3 μM. Compounds 1-3 were screened at 320 non-olfactory GPCRs @Arr2 for agonism. Data is presented as the fold-change from basal relative luminesce units (RLUs) from an average of three assays. GPCRs that induced >3-fold change are labeled, while a comprehensive list of GPCRs was reported previously.⁵⁸

FIG. 8 shows the binding position of 5 with the κOR predicted from alignment with MOM-SalB. The structure of 5 was superimposed onto MOM-SalB bound to the κOR (PDB: 8DZP) by aligning their furan rings and esters. The 5-κOR complex was further energy minimized using Prime (Schrödinger Suite 2023-1). Salt-bridge interactions are shown in yellow. Hydrogen bond interactions shown in pink.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “an excipient” include, but are not limited to, mixtures or combinations of two or more such excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y”’, where ‘x’ and ‘y’ are numerical values, includes “about ‘x’ to about ‘y”’.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range. Thus, for example, if a component is in an amount of about 1%, 2%, 3%, 4%, or 5%, where any value can be a lower and upper endpoint of a range, then any range is contemplated between 1% and 5% (e.g., 1% to 3%, 2% to 4%, etc.).

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “IC₅₀,” is intended to refer to the concentration of a substance (e.g., a compound or a drug) that is required for 50% inhibition of a biological process, or component of a process. For example, IC₅₀ refers to the half maximal (50%) inhibitory concentration (IC) of a substance as determined in a suitable assay.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The position of a substituent can be defined relative to the positions of other substituents in an aromatic ring. For example, as shown below in relationship to the “R” group, a second substituent can be “ortho,” “para,” or “meta” to the R group, meaning that the second substituent is bonded to a carbon labeled ortho, para, or meta as indicated below. Combinations of ortho, para, and meta substituents relative to a given group or substituent are also envisioned and should be considered to be disclosed.

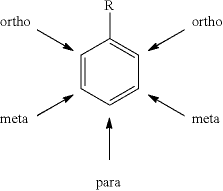

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or -OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. In one aspect, the alkenyl group is a vinyl group or an allyl group. In another aspect, the alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cyclooctynyl, cyclononynyl, and the like. The term “heterocycloalkynyl” is a type of cycloalkenyl group as defined above and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl. Fused aryl groups including, but not limited to, indene and naphthalene groups are also contemplated. The aryl group can be substituted with one or more groups including, but not limited to, alkyl, cycloalkyl, alkoxy, amino, ether, halide, hydroxy, nitro, silyl, sulfo-oxo, or thiol as described herein.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula -NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “acetyloxy” as used herein is represented by the formula —OC(O)CH₃.

The term “amide” or “amido” as used herein is represented by the formula —NHC(O)R, where can be hydrogen, an alkyl group, an aryl group, a heteroaryl group, or a cycloalkyl group as defined herein.

The term “acetyl” as used herein is represented by the formula —C(O)CH₃.

The term “acetyl amino” as used herein is represented by the formula —NHC(O)CH₃.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl. The heteroaryl group can be substituted with one or more groups including, but not limited to, alkyl, cycloalkyl, alkoxy, amino, ether, halide, hydroxy, nitro, silyl, sulfo-oxo, or thiol as described herein.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘},

—O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN;

—N₃; —(CH₂)₀₋₄N(R^{∘})₂; —(CH₂)₀₋₄N(R^{∘})C(O)R^{∘}; —N(R^{∘})C(S)R^{∘};

—(CH₂)₀₋₄N(R^{∘})C(O)NR^{∘}₂; —N(R^{∘})C(S)NR^{∘}₂; —(CH₂)₀₋₄N(R^{∘})C(O)OR^{∘};

—N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘};

—(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘};

—(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂;

—P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}—(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂,

—(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic,

—CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{●} include ═O and ═S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH,

—OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†},

—S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen,

—R^{●}, -(haloR^{●}), —OH, —OR^{●}, —O(haloR^{●}), —CN, —C(O)OH, —C(O)OR^{●}, —NH₂, —NHR^{●}, —NR^{●}₂, or

—NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³¹Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an α-hydrogen can exist in an equilibrium of the keto form and the enol form.

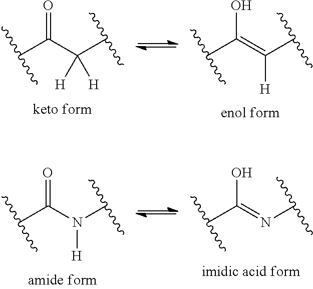 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

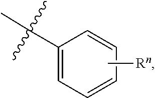

which is understood to be equivalent to a formula:

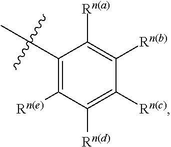

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravitreal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term “treatment” as used herein can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Compounds and Methods of Making and Using the Compounds

In one aspect, disclosed herein is a compound having a structure according to structure I or the pharmaceutically acceptable salt thereof

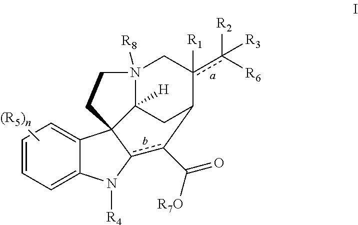

wherein
each [symbol] independently represents a single or double bond;
R₁ is absent when [symbol] is a double bond or R₁ is hydrogen, hydroxyl, or acetyloxy when [symbol] is a single bond;
R₂ is hydrogen;
R₃ is absent when [symbol] is a double bond or is hydrogen, hydroxyl, or acetyloxy when [symbol] is a single bond, or R₂ and R₃ together form a carbonyl group when [symbol] is a single bond;
R₄ is hydrogen, substituted or unsubstituted alkyl, or aralkyl;
R₆ and R₇ are alkyl; and
R₅ is selected from hydrogen, hydroxy, alkoxy, hydroxyalkyl, alkoxyalkyl, halide, haloalkyl, nitrile, nitro, amino, amido, acetyl, acetylamino, acetyloxy, substituted or unsubstituted alkyl, substituted or unsubstituted cycloalkyl, substituted or unsubstituted heterocycloalkyl, substituted or unsubstituted aryl, or substituted or unsubstituted heteroaryl;
n is 1, 2, 3, or 4; and
R₈ is absent or is a substituted or unsubstituted alkyl, an alkenyl group, or an aralkyl group

In one aspect, [symbol] in structure I is a double bond. In another aspect, R₇ in structure I is a C1 to C5 alkyl group. In another aspect, R₇ in structure I is a methyl group. In another aspect, R₄ in structure I is hydrogen. In another aspect, [symbol] in structure I is a double bond. In another aspect, R₆ in structure I is a C1 to C5 alkyl group. In another aspect, R₆ in structure I is a methyl group. In another aspect, [symbol] in structure I is a single bond. In another aspect, R₆ in structure I is a C1 to C5 alkyl group. In another aspect, R₆ in structure I is a methyl group. In another aspect, R₃ in structure I is hydroxyl. In another aspect, when [symbol] in structure I is a single bond and R₂ and R₃ together form a carbonyl group. In another aspect, R₆ in structure I is a C1 to C5 alkyl group. In another aspect, R₆ in structure I is a methyl group. In another aspect, R₁ in structure I is acetyloxy. In another aspect, R₈ in structure I is absent. In another aspect, n in structure I is 1.

In one aspect, the compound has the structure II or the pharmaceutically acceptable salt thereof

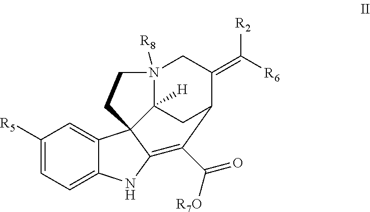

In one aspect, R₈ in structure II is absent or a substituted or unsubstituted alkyl, an alkenyl group, or an aralkyl group and R₇ is a C1 to C5 alkyl group. In another aspect, R₈ in structure II is absent and R₇ is a methyl group. In another aspect, R₂ in structure II is hydrogen and R₆ is a C1 to C5 alkyl group. In another aspect, R₂ in structure II is hydrogen and R₆ is a methyl group. In another aspect, R₅ in structure II is selected from hydrogen, hydroxy, alkoxy, hydroxyalkyl, alkoxyalkyl, halide, haloalkyl, nitrile, nitro, amino, amido, acetyl, acetylamino, acetyloxy, substituted or unsubstituted alkyl, substituted or unsubstituted cycloalkyl, substituted or unsubstituted heterocycloalkyl, substituted or unsubstituted aryl, or substituted or unsubstituted heteroaryl.

In one aspect, the compound has the structure Ill or the pharmaceutically acceptable salt thereof

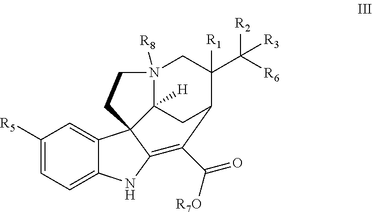

In one aspect, R₈ in structure Ill is absent or a substituted or unsubstituted alkyl, an alkenyl group, or an aralkyl group and R₇ is a C1 to C5 alkyl group. In another aspect, R₈ in structure Ill is absent and R₇ is a methyl group. In another aspect, R₂ in structure Ill is hydrogen, R₃ is hydrogen or hydroxyl, and R₆ is a C1 to C5 alkyl group. In another aspect, R₂ in structure Ill is hydrogen, R₃ is hydrogen or hydroxyl, and R₆ is a methyl group. In another aspect, R₂ and R₃ in structure Ill together form a carbonyl group and R₆ is a C1 to C5 alkyl group. In another aspect, R₂ and R₃ in structure Ill together form a carbonyl group and R₆ is a methyl group. In another aspect, R₁ in structure Ill is hydrogen, hydroxyl, or acetyloxy. In another aspect, R₅ in structure Ill is selected from hydrogen, hydroxy, alkoxy, hydroxyalkyl, alkoxyalkyl, halide, haloalkyl, nitrile, nitro, amino, amido, acetyl, acetylamino, acetyloxy, substituted or unsubstituted alkyl, substituted or unsubstituted cycloalkyl, substituted or unsubstituted heterocycloalkyl, substituted or unsubstituted aryl, or substituted or unsubstituted heteroaryl.

In one aspect, the compound has the formula V:

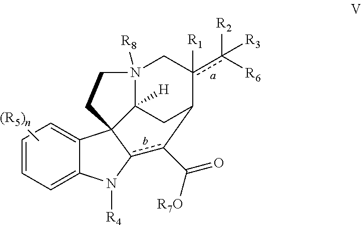

or a pharmaceutically acceptable salt thereof,
wherein
each [symbol] independently represents a single or double bond;
R₁ is absent when [symbol] is a double bond or R₁ is hydrogen, hydroxyl, or acetyloxy when [symbol] is a single bond;
R₂ is hydrogen;
R₃ is absent when [symbol] is a double bond or is hydrogen, hydroxyl, or acetyloxy when [symbol] is a single bond, or R₂ and R₃ together form a carbonyl group when [symbol] is a single bond;
R₄ is hydrogen, substituted or unsubstituted alkyl, or aralkyl;
R₆ and R₇ are alkyl; and
R₅ is selected from hydrogen, hydroxy, alkoxy, hydroxyalkyl, alkoxyalkyl, halide, haloalkyl, nitrile, nitro, amino, amido, acetyl, acetylamino, acetyloxy, substituted or unsubstituted alkyl, substituted or unsubstituted cycloalkyl, substituted or unsubstituted heterocycloalkyl, substituted or unsubstituted aryl, or substituted or unsubstituted heteroaryl, wherein at least one of R₅ is not hydrogen;
n is 1, 2, 3, or 4; and
R₈ is absent or is a substituted or unsubstituted alkyl, an alkenyl group, a cycloalkyl group, or an aralkyl group,
provided the compound of formula I does not include:

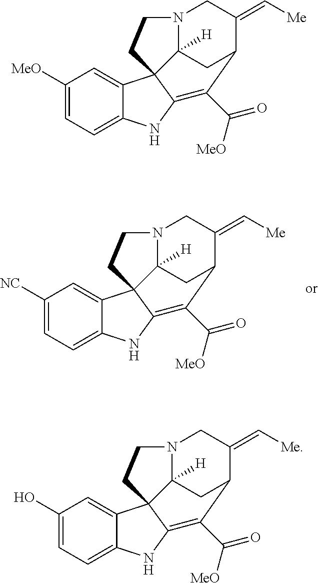

Referring to formula V, at least one of R₅ is not hydrogen. In other words, the phenyl ring of formula V is substituted with at least one group other than hydrogen.

In one aspect, the compound has the formula VI:

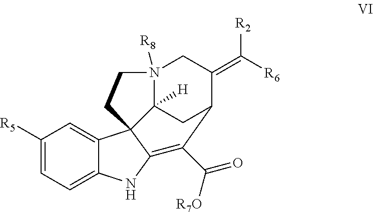

or a pharmaceutically acceptable salt thereof,
wherein
   R₂ is hydrogen;
   R₆ and R₇ are alkyl; and
   R₅ is selected from hydroxy, alkoxy, hydroxyalkyl, alkoxyalkyl, halide, haloalkyl, nitrile, nitro, amino, amido, acetyl, acetylamino, acetyloxy, substituted or unsubstituted alkyl, substituted or unsubstituted cycloalkyl, substituted or unsubstituted heterocycloalkyl, substituted or unsubstituted aryl, or substituted or unsubstituted heteroaryl; and
   R₈ is absent or is a substituted or unsubstituted alkyl, an alkenyl group, a cycloalkyl group, or an aralkyl group.

In one aspect, the compound has the formula VII:

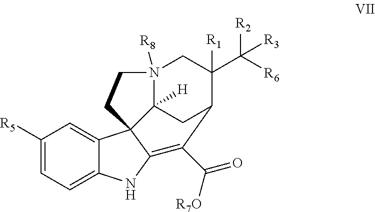

wherein R₁-R₃ and R₅-R₈ are as defined above.

In one aspect, the compound has the formula VIII:

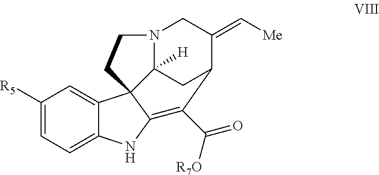

or a pharmaceutically acceptable salt thereof,
wherein
   R₇ is alkyl; and
   R₅ is selected from halide, substituted or unsubstituted heteroaryl, or substituted or unsubstituted heteroaryl.

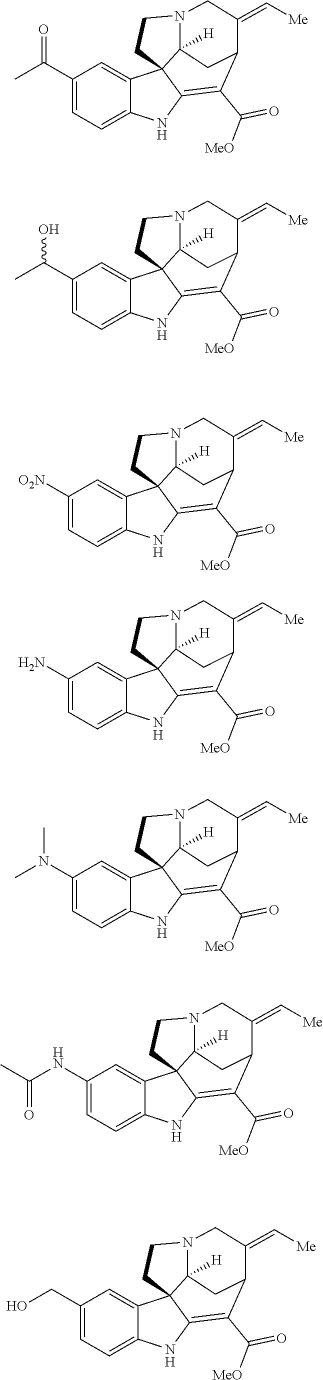

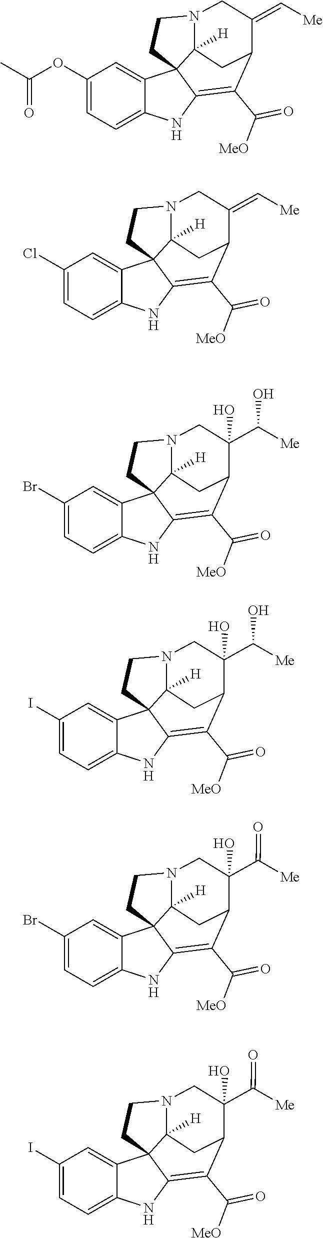

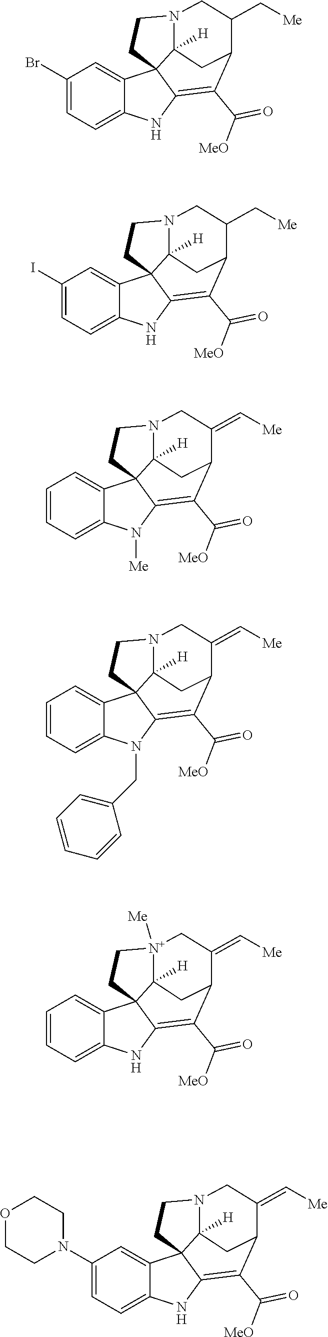

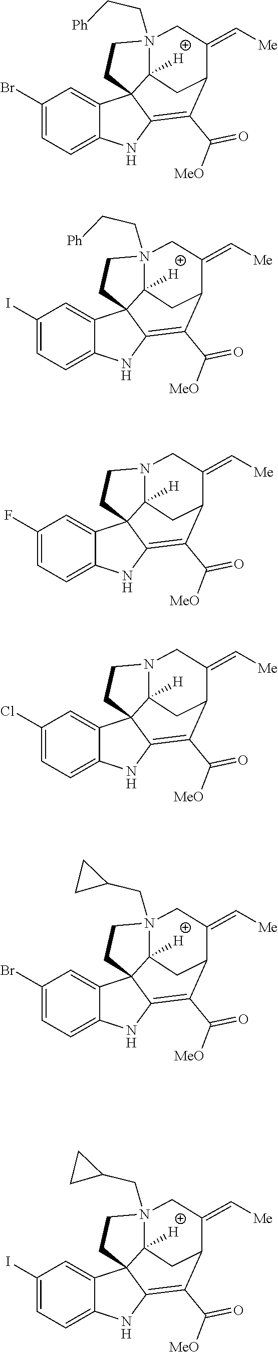

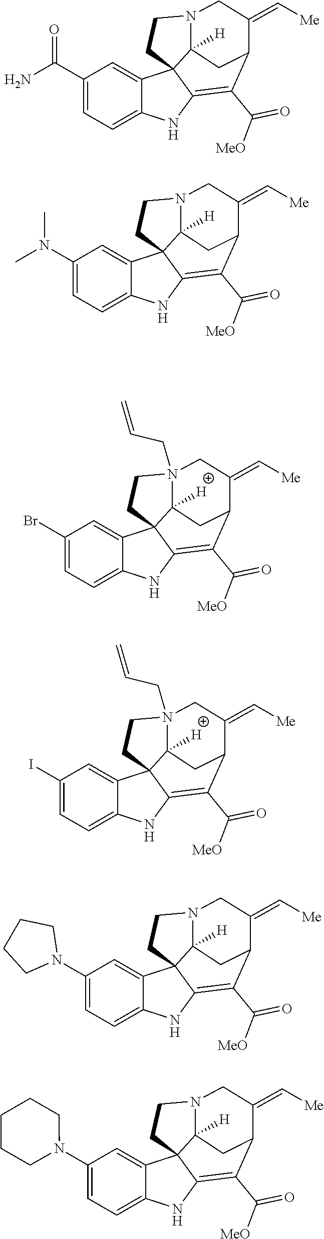

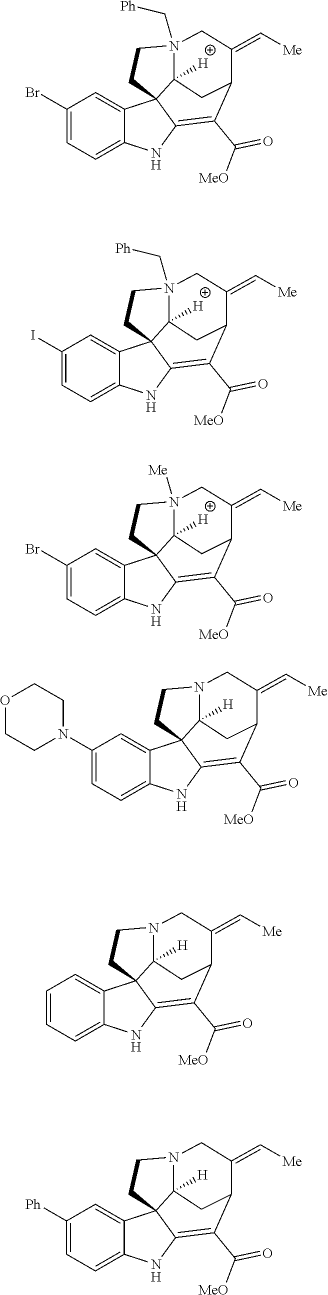

In another aspect, the compound is

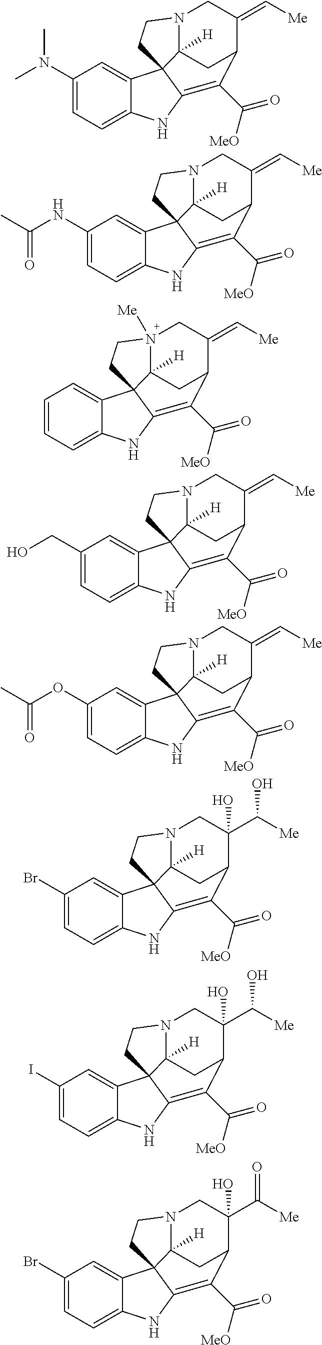

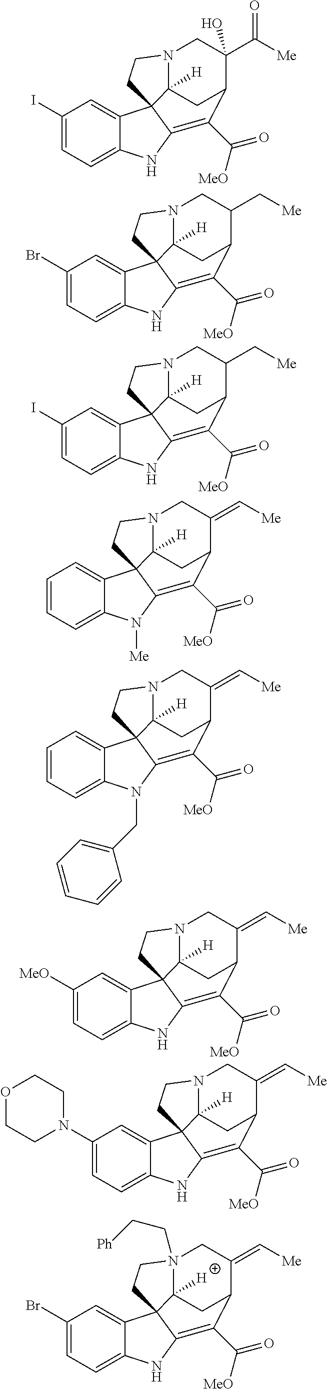

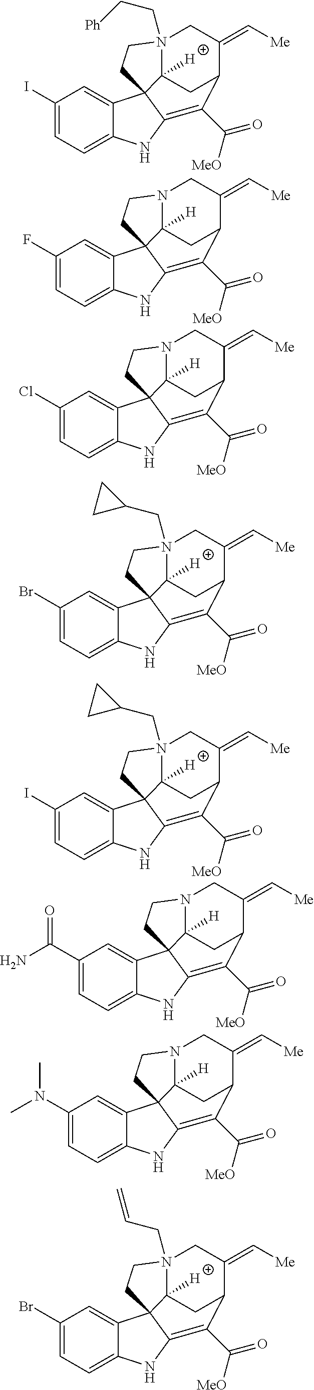

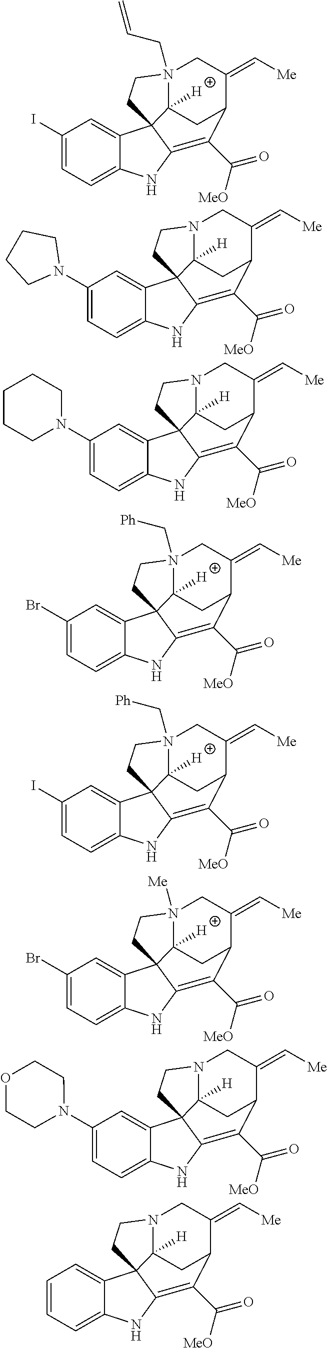

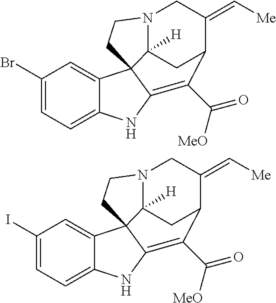

In one aspect, the compound is not

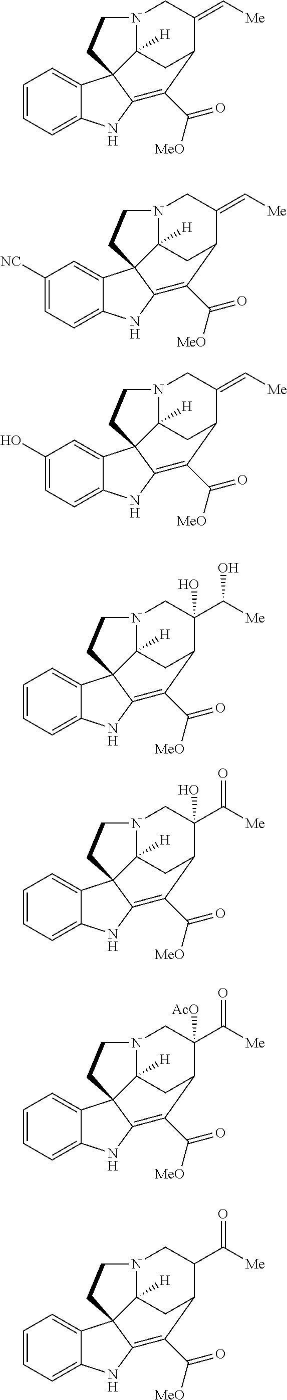

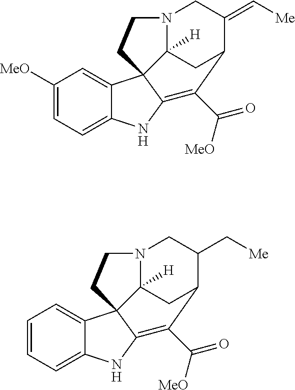

In another aspect, the compound is not

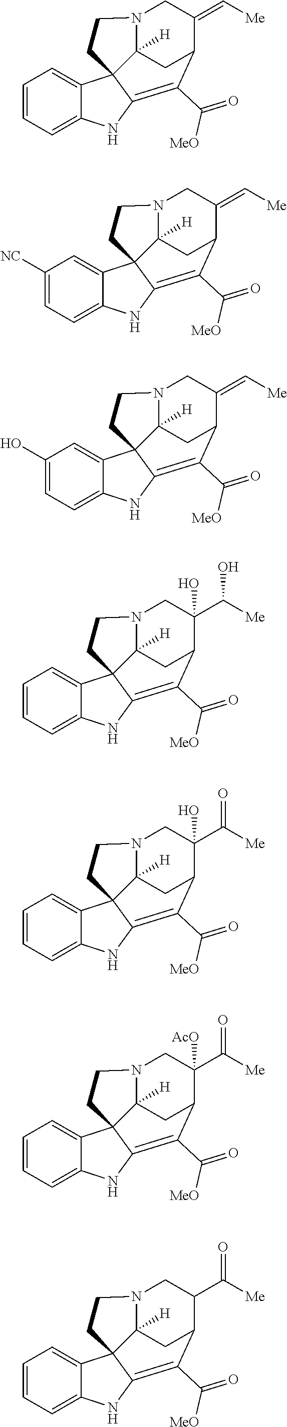

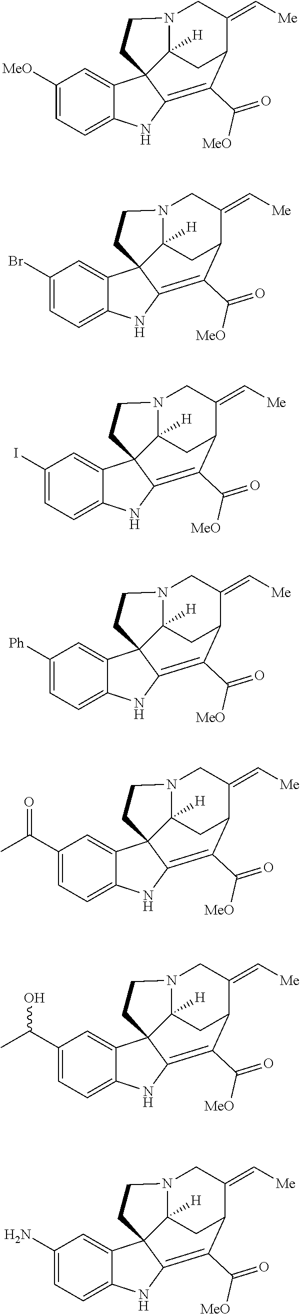

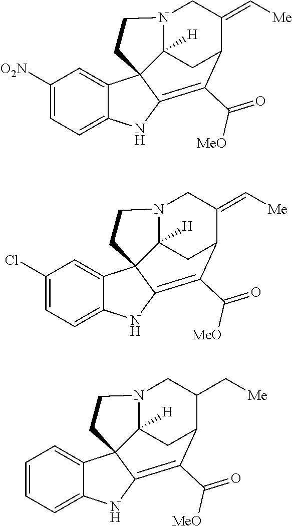

In another aspect, the compound is not

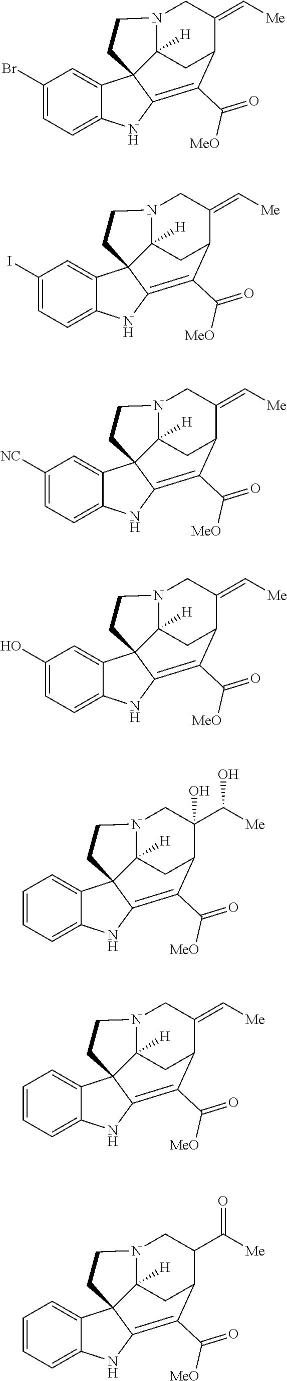

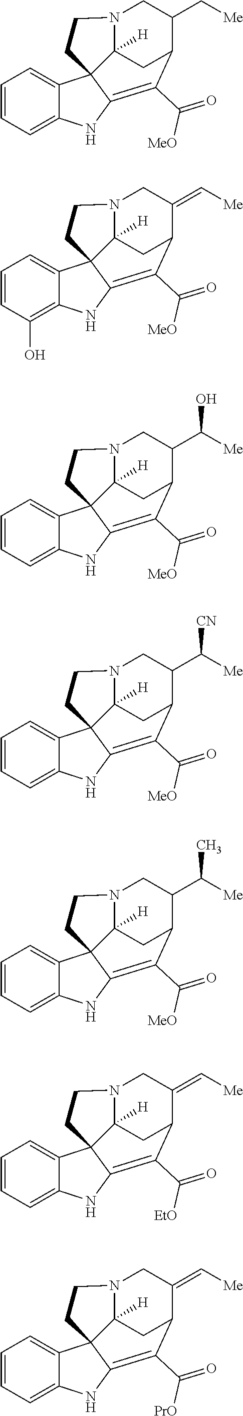

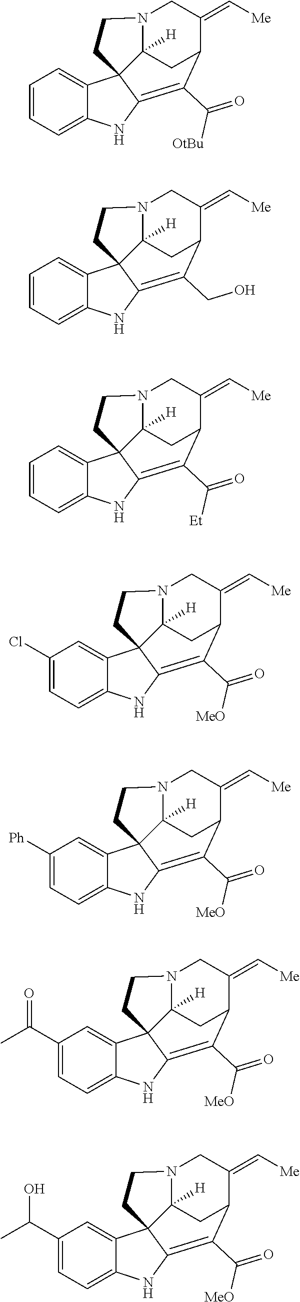

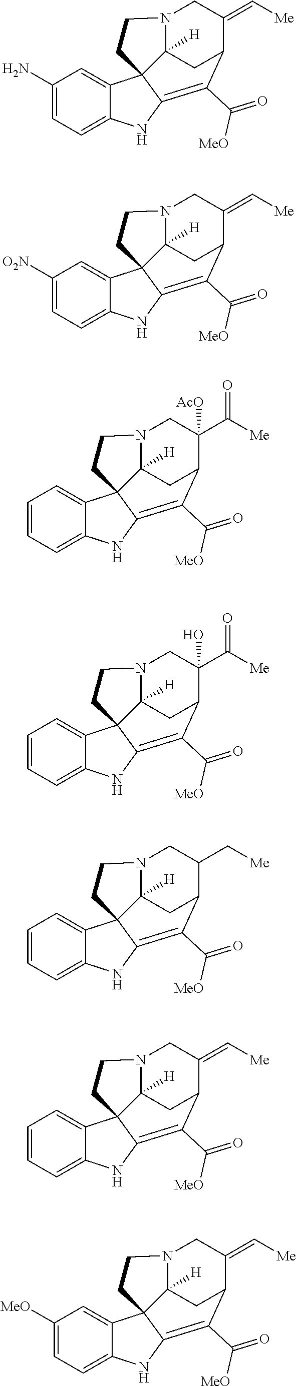

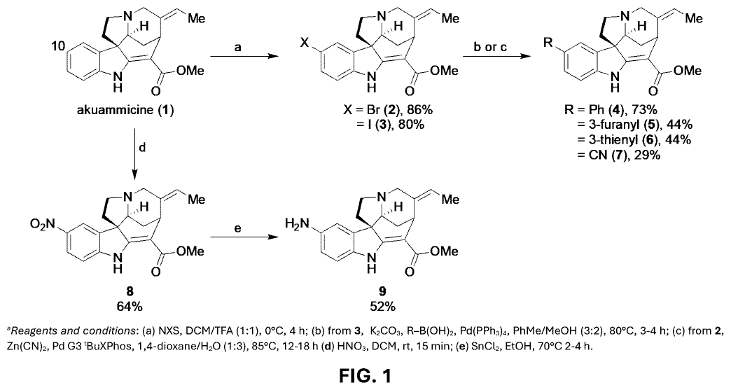

Exemplary methods for producing compounds described herein, as well as characterization information, are provided in the Examples. For example, FIGS. 1-4 provide synthetic procedures for making a variety of different compounds described herein. Solvents, temperatures, presence or absence of protecting groups, and other reaction conditions may vary according to the specific substituents in the compound being synthesized.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethyl carbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In one aspect, an appropriate dosage level will generally be about 0.01 to 1000 mg of a compound described herein per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

### Methods of Use

Classical opioid analgesics, including morphine, mediate all of their desired and undesired effects by specific activation of the μ-opioid receptor (μ receptor). The use of morphine for treating chronic pain, however, is limited by the development of constipation, respiratory depression, tolerance and dependence. Analgesic effects can also be mediated through other members of the opioid receptor family such as the κ-Opioid receptor (κ receptor), δ-opioid receptor (δ receptor) and the nociceptin/orphanin FQ peptide receptor (NOP receptor).

In one aspect, the compounds described herein can bind to the kappa opioid receptor (κOR) and behave as an agonist. As shown in the Examples, compounds described herein demonstrate the ability of the compounds described herein to bind to κOR. The ability of the compounds to bind to opioid receptors make them effective in the treatment or prevention of pain. In one aspect, the pain can be chronic or acute pain.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

Aspect 1. A compound of formula I

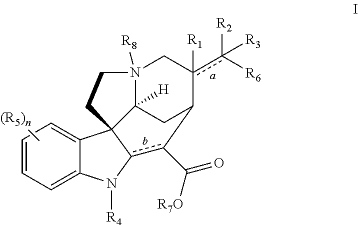

or a pharmaceutically acceptable salt thereof,
wherein
each [symbol] independently represents a single or double bond;
R₁ is absent when [symbol] is a double bond or R₁ is hydrogen, hydroxyl, or acetyloxy when [symbol] is a single bond;
R₂ is hydrogen;
R₃ is absent when [symbol] is a double bond or is hydrogen, hydroxyl, or acetyloxy when [symbol] is a single bond, or R₂ and R₃ together form a carbonyl group when [symbol] is a single bond;
R₄ is hydrogen, substituted or unsubstituted alkyl, or aralkyl;
R₆ and R₇ are alkyl; and
R₅ is selected from hydrogen, hydroxy, alkoxy, hydroxyalkyl, alkoxyalkyl, halide, haloalkyl, nitrile, nitro, amino, amido, acetyl, acetylamino, acetyloxy, substituted or unsubstituted alkyl, substituted or unsubstituted cycloalkyl, substituted or unsubstituted heterocycloalkyl, substituted or unsubstituted aryl, or substituted or unsubstituted heteroaryl;
n is 1, 2, 3, or 4; and
R₈ is absent or is a substituted or unsubstituted alkyl, an alkenyl group, or an aralkyl group.

Aspect 2. The compound of Aspect 1, wherein when [symbol] is a double bond.

Aspect 3. The compound of Aspect 1 or 2, wherein R₇ is a C1 to C5 alkyl group.

Aspect 4. The compound of Aspect 1 or 2, wherein R₇ is a methyl group.

Aspect 5. The compound of any one of Aspects 1-4, wherein R₄ is hydrogen.

Aspect 6. The compound of any one of Aspects 1-5, wherein when [symbol] is a double bond.

Aspect 7. The compound of Aspect 6, wherein R₆ is a C1 to C5 alkyl group.

Aspect 8. The compound of Aspect 6, wherein R₆ is a methyl group.

Aspect 9. The compound of any one of Aspects 1-5, wherein when [symbol] is a single bond.

Aspect 10. The compound of Aspect 9, wherein R₆ is a C1 to C5 alkyl group.

Aspect 11. The compound of Aspect 9, wherein R₆ is a methyl group.

Aspect 12. The compound of any one of Aspects 9-11, wherein R₃ is hydroxyl.

Aspect 13. The compound of any one of Aspects 1-5, wherein when [symbol] is a single bond and R₂ and R₃ together form a carbonyl group.

Aspect 14. The compound of Aspect 13, wherein R₆ is a C1 to C5 alkyl group.

Aspect 15. The compound of Aspect 13, wherein R₆ is a methyl group.

Aspect 16. The compound of any one of Aspects 13-15, wherein R₁ is acetyloxy.

Aspect 17. The compound of any one of Aspects 1-16, wherein R₈ is absent.

Aspect 18. The compound of any one of Aspects 1-17, wherein n is 1.

Aspect 19. The compound of Aspect 1, wherein the compound is formula II

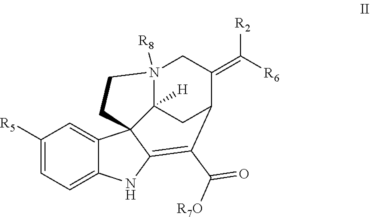

Aspect 20. The compound of Aspect 19, wherein R₈ is absent or a substituted or unsubstituted alkyl, an alkenyl group, or an aralkyl group and R₇ is a C1 to C5 alkyl group.

Aspect 21. The compound of Aspect 19, wherein R₈ is absent and R₇ is a methyl group.

Aspect 22. The compound of any one of Aspects 19-21, wherein and R₂ is hydrogen and R₆ is a C1 to C5 alkyl group.

Aspect 23. The compound of any one of Aspects 19-21, wherein and R₂ is hydrogen and R₆ is a methyl group.

Aspect 24. The compound of any one of Aspects 19-23, wherein R₅ is selected from hydrogen, hydroxy, alkoxy, hydroxyalkyl, alkoxyalkyl, halide, haloalkyl, nitrile, nitro, amino, amido, acetyl, acetylamino, acetyloxy, substituted or unsubstituted alkyl, substituted or unsubstituted cycloalkyl, substituted or unsubstituted heterocycloalkyl, substituted or unsubstituted aryl, or substituted or unsubstituted heteroaryl.

Aspect 25. The compound of Aspect 1, wherein the compound is formula III

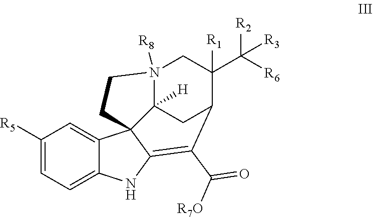

Aspect 26. The compound of Aspect 25, wherein R₈ is absent or a substituted or unsubstituted alkyl, an alkenyl group, or an aralkyl group and R₇ is a C1 to C5 alkyl group.

Aspect 27. The compound of Aspect 25, wherein R₈ is absent and R₇ is a methyl group.

Aspect 28. The compound of any one of Aspects 25-27, wherein and R₂ is hydrogen, R₃ is hydrogen or hydroxyl, and R₆ is a C1 to C5 alkyl group.

Aspect 29. The compound of any one of Aspects 25-27, wherein and R₂ is hydrogen, R₃ is hydrogen or hydroxyl, and R₆ is a methyl group.

Aspect 30. The compound of any one of Aspects 25-27, wherein R₂ and R₃ together form a carbonyl group and R₆ is a C1 to C5 alkyl group.

Aspect 31. The compound of any one of Aspects 25-27, wherein R₂ and R₃ together form a carbonyl group and R₆ is a methyl group.

Aspect 32. The compound of any one of Aspects 25-31, wherein R₁ is hydrogen, hydroxyl, or acetyloxy.

Aspect 33. The compound of any one of Aspects 25-32, wherein R₅ is selected from hydrogen, hydroxy, alkoxy, hydroxyalkyl, alkoxyalkyl, halide, haloalkyl, nitrile, nitro, amino, amido, acetyl, acetylamino, acetyloxy, substituted or unsubstituted alkyl, substituted or unsubstituted cycloalkyl, substituted or unsubstituted heterocycloalkyl, substituted or unsubstituted aryl, or substituted or unsubstituted heteroaryl.

Aspect 34. The compound of Aspect 1 selected from

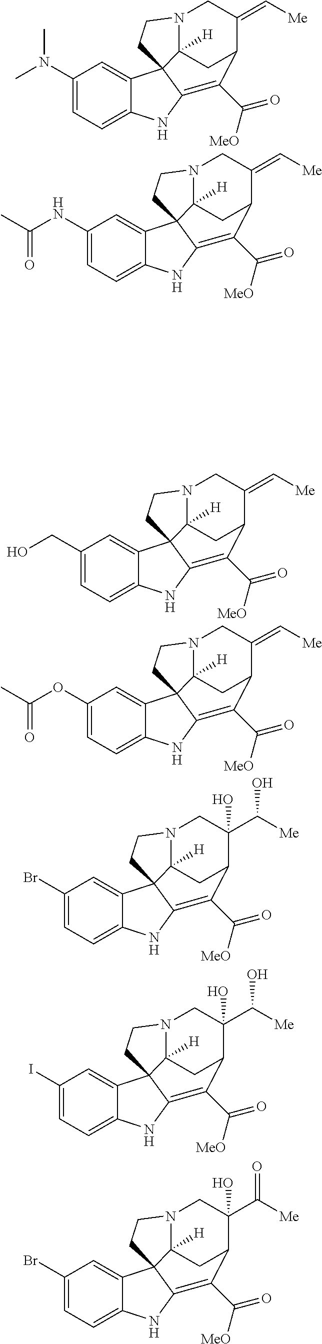

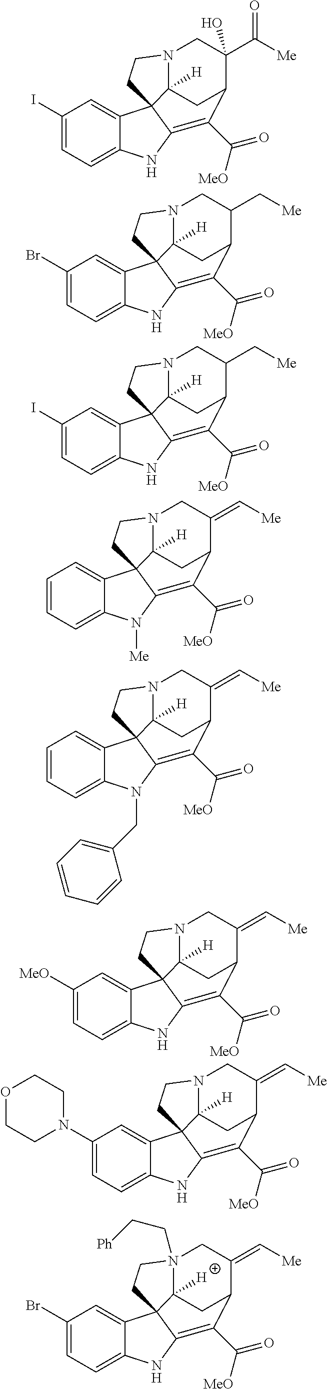

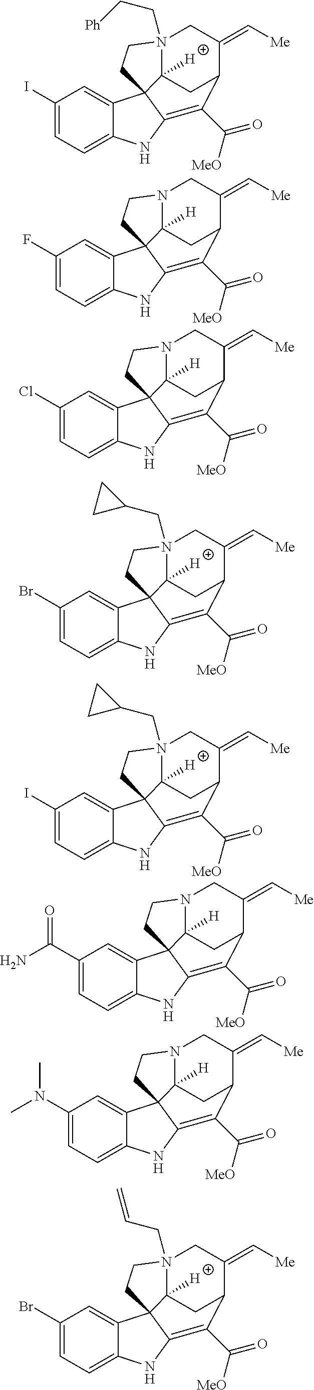

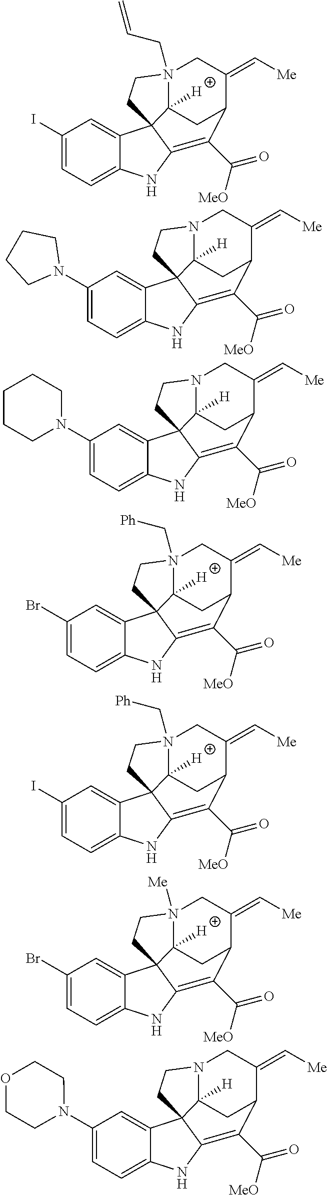

Aspect 35. The compound of Aspect 1 selected from

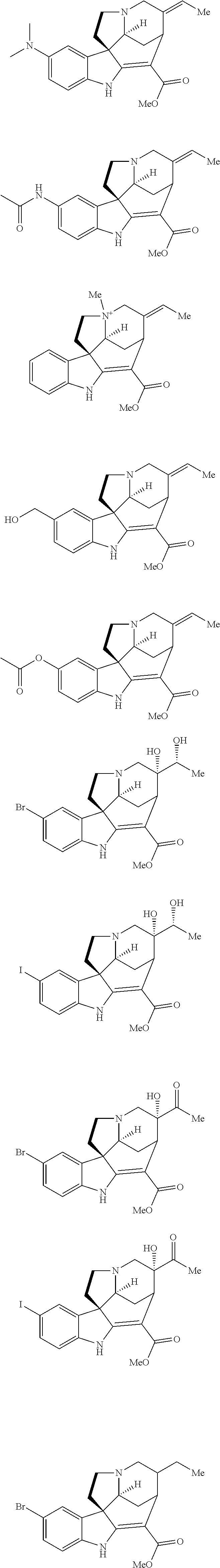

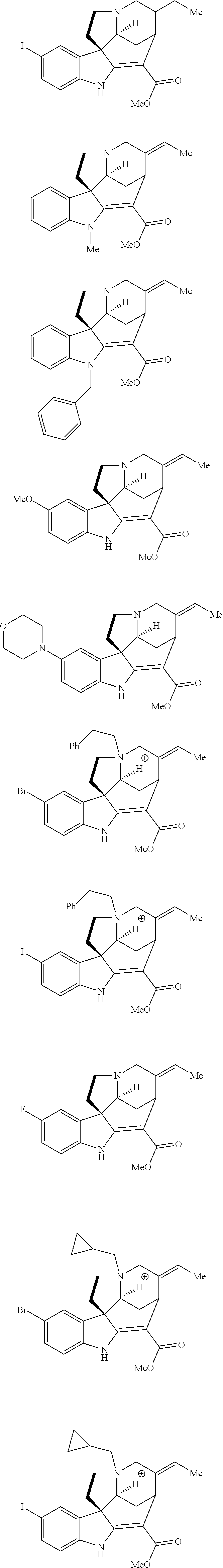

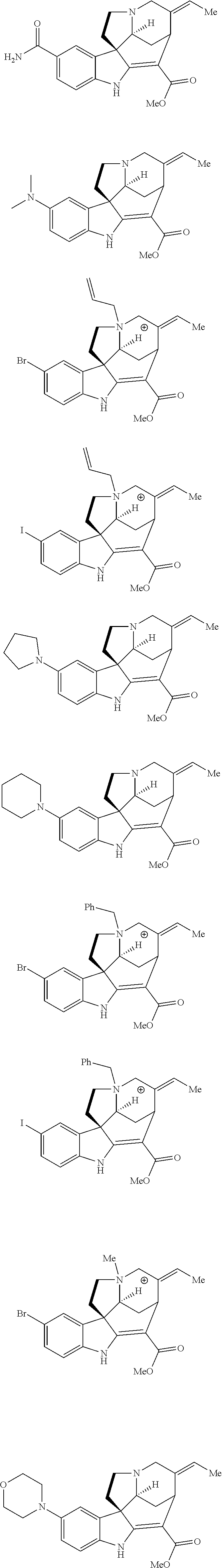

Aspect 36. The compound of Aspect 1, wherein the compound is not

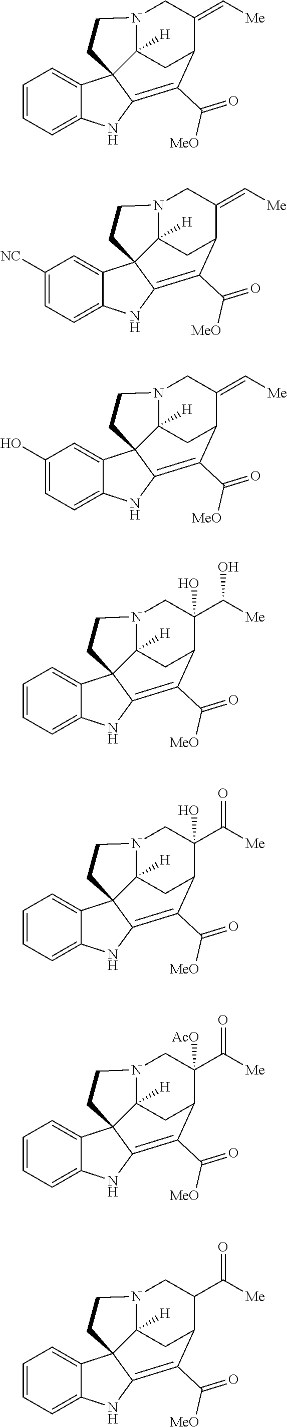

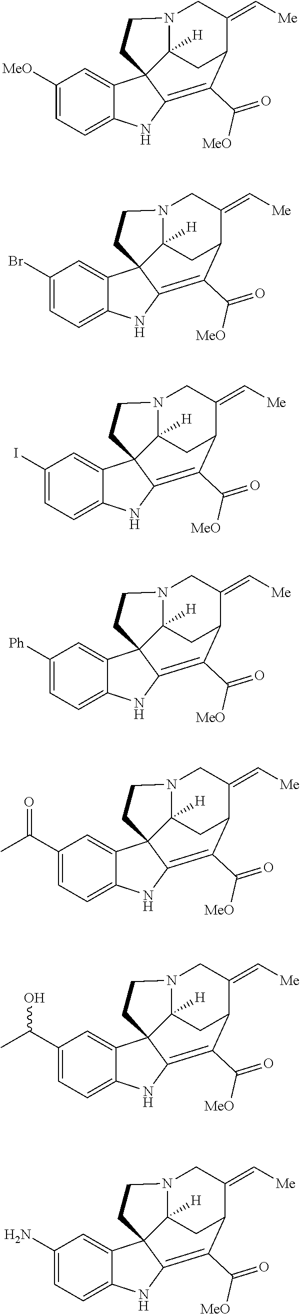

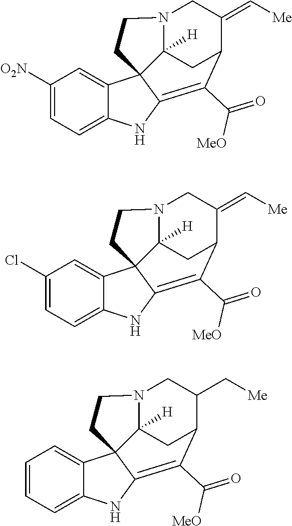

Aspect 37. The compound of Aspect 1, wherein the compound is not

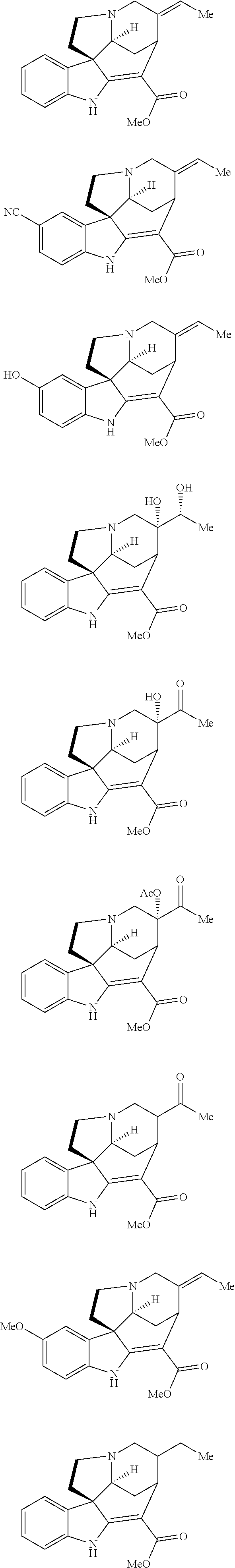

Aspect 38. A pharmaceutical composition comprising the compound of any one of Aspects 1-37 and a pharmaceutically acceptable carrier.

Aspect 39. A method of treating or preventing pain in a subject comprising administering to the subject a compound of any one of Aspects 1-37.

Aspect 40. The method of Aspect 39, wherein the subject has chronic pain.

Aspect 41. A compound of formula V

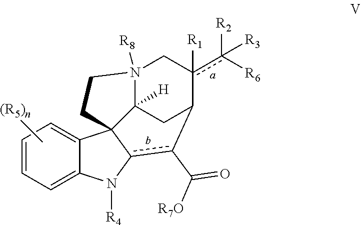

or a pharmaceutically acceptable salt thereof,
wherein
   each [symbol] independently represents a single or double bond;
   R₁ is absent when [symbol] is a double bond or R₁ is hydrogen, hydroxyl, or acetyloxy when
   [symbol] is a single bond;
   R₂ is hydrogen;
   R₃ is absent when [symbol] is a double bond or is hydrogen, hydroxyl, or acetyloxy when [symbol] is a single bond, or R₂ and R₃ together form a carbonyl group when [symbol] is a single bond;
   R₄ is hydrogen, substituted or unsubstituted alkyl, or aralkyl;
   R₆ and R₇ are alkyl; and
   R₅ is selected from hydrogen, hydroxy, alkoxy, hydroxyalkyl, alkoxyalkyl, halide, haloalkyl, nitrile, nitro, amino, amido, acetyl, acetylamino, acetyloxy, substituted or unsubstituted alkyl, substituted or unsubstituted cycloalkyl, substituted or unsubstituted heterocycloalkyl, substituted or unsubstituted aryl, or substituted or unsubstituted heteroaryl, wherein at least one of R₅ is not hydrogen;
   n is 1, 2, 3, or 4; and
   R₈ is absent or is a substituted or unsubstituted alkyl, an alkenyl group, a cycloalkyl group, or an aralkyl group,
   provided the compound of formula I does not include:

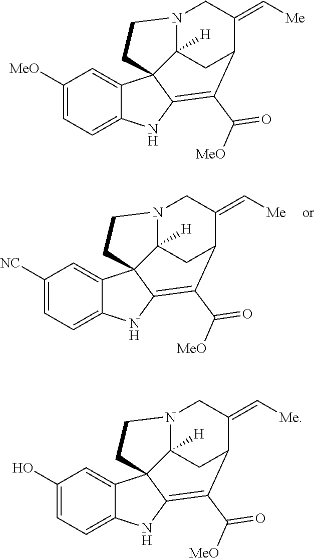

Aspect 42. The compound of Aspect 41, wherein [symbol] is a double bond.

[symbol]

Aspect 43. The compound of Aspect 41 or 42, wherein R₇ is a C1 to C5 alkyl group.

Aspect 44. The compound of Aspect 41 or 42, wherein R₇ is a methyl group.

Aspect 45. The compound of any one of Aspects 41-44, wherein R₄ is hydrogen.

Aspect 46. The compound of any one of Aspects 41-45, wherein when [symbol] is a double bond.

Aspect 47. The compound of Aspect 46, wherein R₆ is a C1 to C5 alkyl group.

Aspect 48. The compound of Aspect 6, wherein R₆ is a methyl group.

Aspect 49. The compound of any one of Aspects 41-45, wherein when [symbol] is a single bond.

Aspect 50. The compound of Aspect 49, wherein R₆ is a C1 to C5 alkyl group.

Aspect 51. The compound of Aspect 49, wherein R₆ is a methyl group.

Aspect 52. The compound of Aspect 49, wherein R₃ is hydroxyl.

Aspect 53. The compound of Aspect 41, wherein when [symbol] is a single bond and R₂ and R₃ together form a carbonyl group.

Aspect 54. The compound of Aspect 53, wherein R₆ is a C1 to C5 alkyl group.

Aspect 55. The compound of Aspect 53, wherein R₆ is a methyl group.

Aspect 56. The compound of any one of Aspects 53-55, wherein R₁ is acetyloxy.

Aspect 57. The compound of any one of Aspects 41-56, wherein R₈ is absent.

Aspect 58. The compound of any one of Aspects 41-57, wherein n is 1.

Aspect 59. The compound of Aspect 41, wherein the compound is formula VI

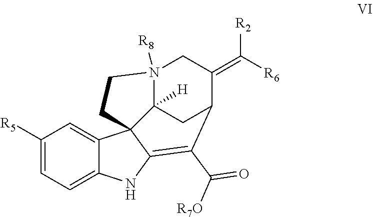

or a pharmaceutically acceptable salt thereof,
wherein
R₂ is hydrogen;
R₆ and R₇ are alkyl; and
R₅ is selected from hydroxy, alkoxy, hydroxyalkyl, alkoxyalkyl, halide, haloalkyl, nitrile, nitro, amino, amido, acetyl, acetylamino, acetyloxy, substituted or unsubstituted alkyl, substituted or unsubstituted cycloalkyl, substituted or unsubstituted heterocycloalkyl, substituted or unsubstituted aryl, or substituted or unsubstituted heteroaryl; and
R₈ is absent or is a substituted or unsubstituted alkyl, an alkenyl group, a cycloalkyl group, or an aralkyl group.

Aspect 60. The compound of Aspect 59, wherein R₇ is a C1 to C5 alkyl group.

Aspect 61. The compound of Aspect 59, wherein R₈ is absent and R₇ is a methyl group.

Aspect 62. The compound of any one of Aspects 59-61, wherein and R₂ is hydrogen and R₆ is a C1 to C5 alkyl group.

Aspect 63. The compound of any one of Aspects 59-61, wherein and R₂ is hydrogen and R₆ is a methyl group.

Aspect 64. The compound of any one of Aspects 59-63, wherein R₅ is a halide or a substituted or unsubstituted heteroaryl.

Aspect 65. The compound of Aspect 41, wherein the compound is formula VII

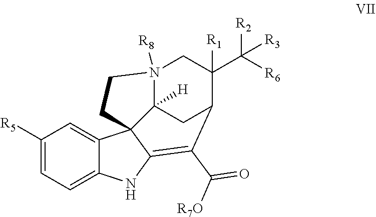

Aspect 66. The compound of Aspect 65, wherein R₈ is absent or a substituted or unsubstituted alkyl, an alkenyl group, or an aralkyl group and R₇ is a C1 to C5 alkyl group.

Aspect 67. The compound of Aspect 65, wherein R₈ is absent and R₇ is a methyl group.

Aspect 68. The compound of any one of Aspects 65-67, wherein and R₂ is hydrogen, R₃ is hydrogen or hydroxyl, and R₆ is a C1 to C5 alkyl group.

Aspect 69. The compound of any one of Aspects 65-67, wherein and R₂ is hydrogen, R₃ is hydrogen or hydroxyl, and R₆ is a methyl group.

Aspect 70. The compound of any one of Aspects 65-67, wherein R₂ and R₃ together form a carbonyl group and R₆ is a C1 to C5 alkyl group.

Aspect 71. The compound of any one of Aspects 65-67, wherein R₂ and R₃ together form a carbonyl group and R₆ is a methyl group.

Aspect 72. The compound of any one of Aspects 65-71, wherein R₁ is hydrogen, hydroxyl, or acetyloxy.

Aspect 73. The compound of any one of Aspects 65-72, wherein R₅ is selected from hydrogen, hydroxy, alkoxy, hydroxyalkyl, alkoxyalkyl, halide, haloalkyl, nitrile, nitro, amino, amido, acetyl, acetylamino, acetyloxy, substituted or unsubstituted alkyl, substituted or unsubstituted cycloalkyl, substituted or unsubstituted heterocycloalkyl, substituted or unsubstituted aryl, or substituted or unsubstituted heteroaryl.

Aspect 74. The compound of Aspect 41, wherein the compound is formula VIII

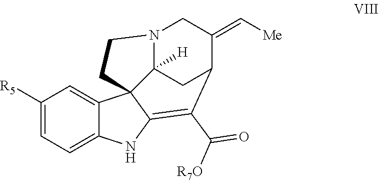

or a pharmaceutically acceptable salt thereof,
wherein
   R₇ is alkyl; and
   R₅ is selected from halide, substituted or unsubstituted heteroaryl, or substituted or unsubstituted heteroaryl.

Aspect 75. The compound of Aspect 41 selected from

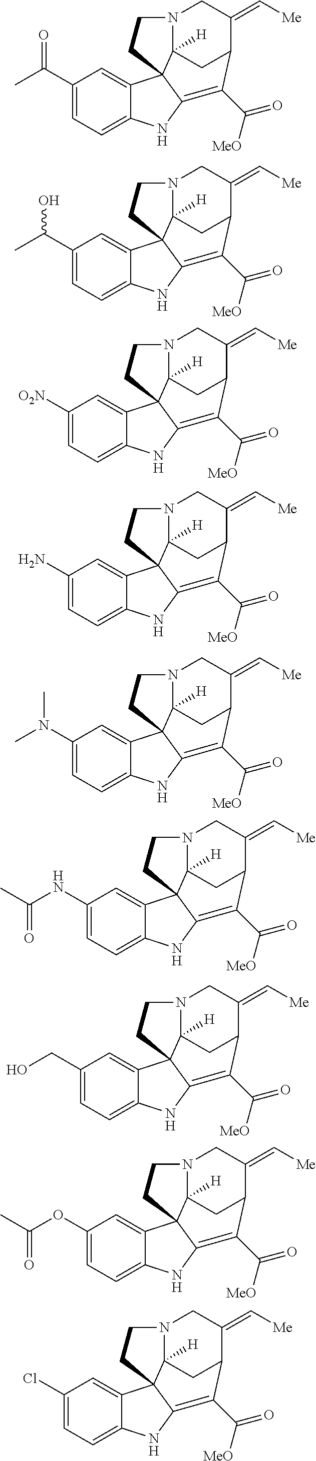

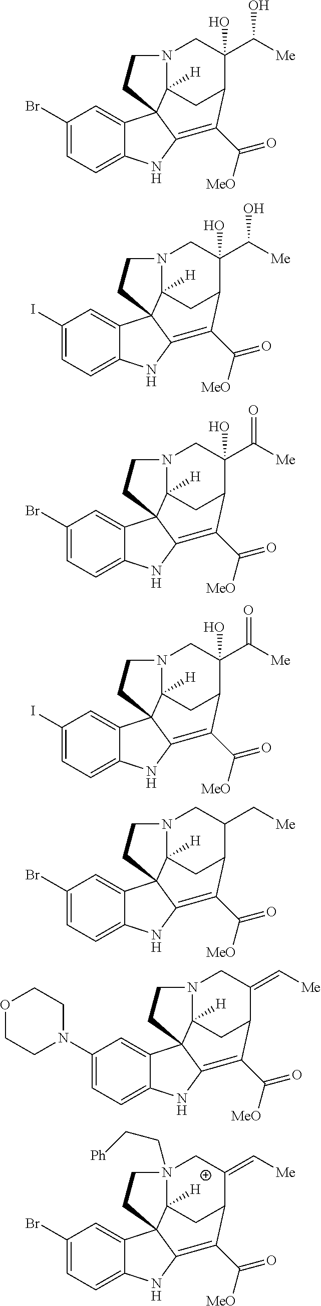

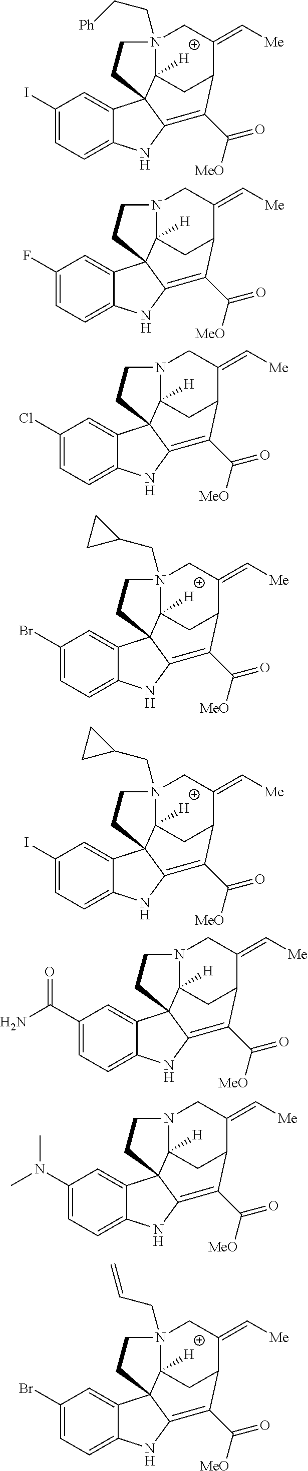

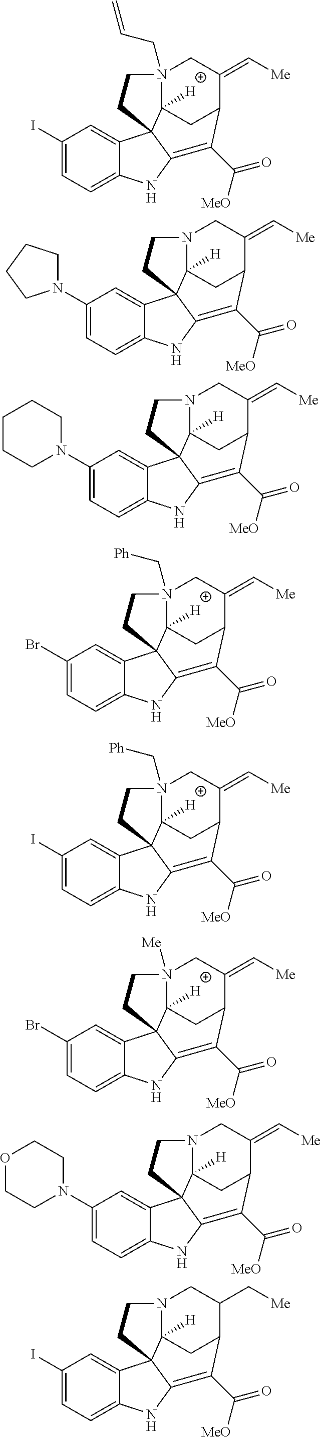

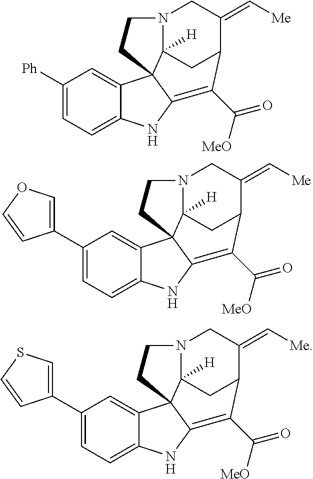

Aspect 76. The compound of Aspect 41 selected from

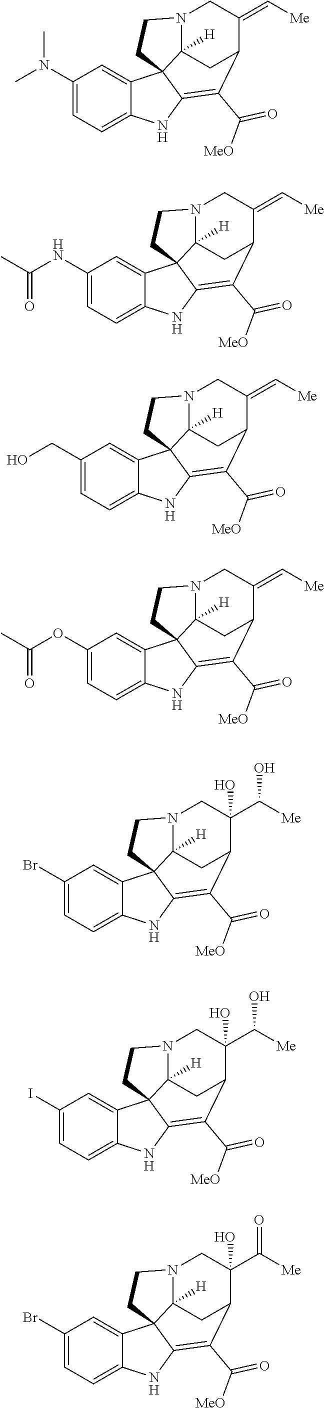

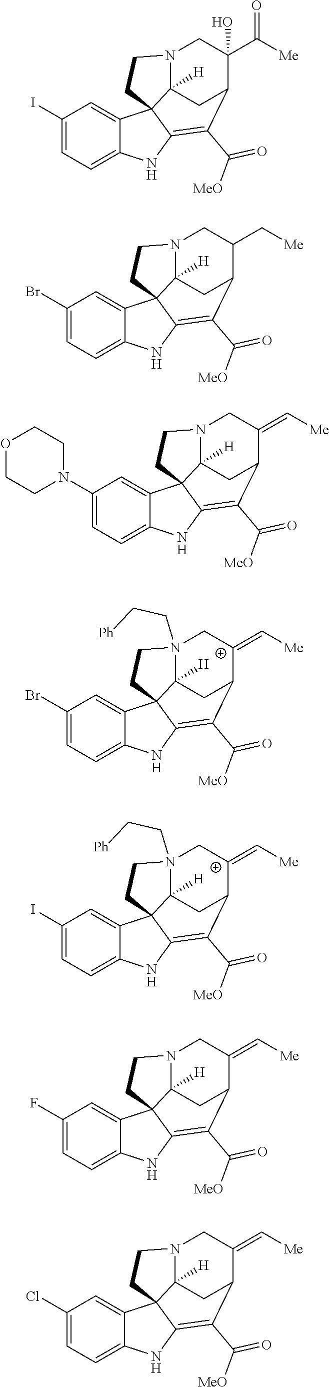

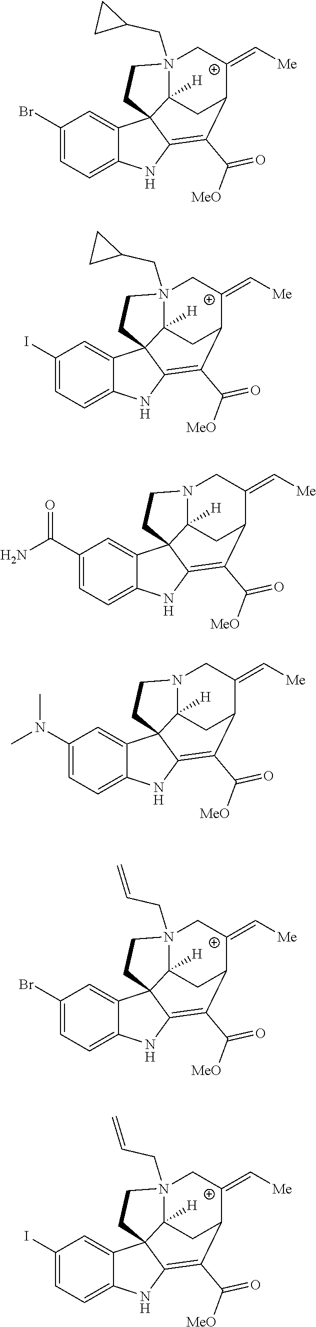

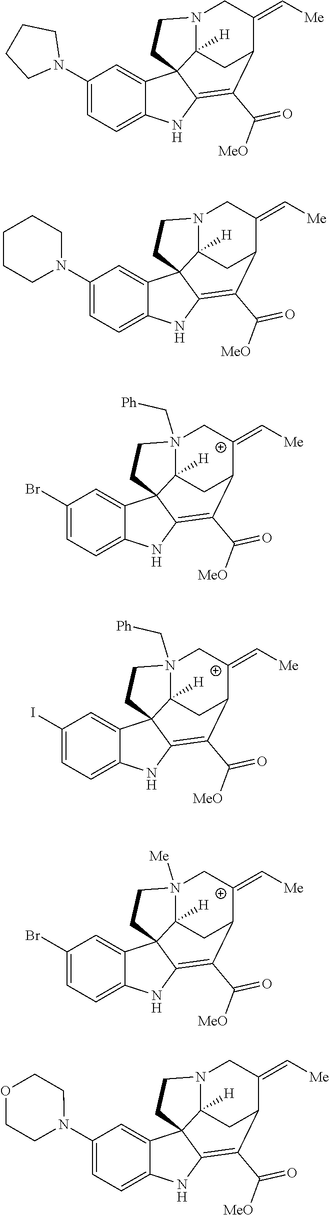

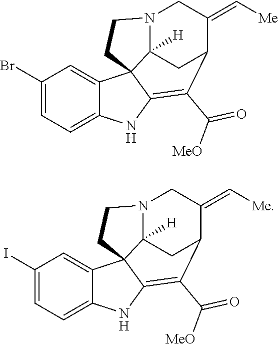

Aspect 77. The compound of Aspect 41 selected from

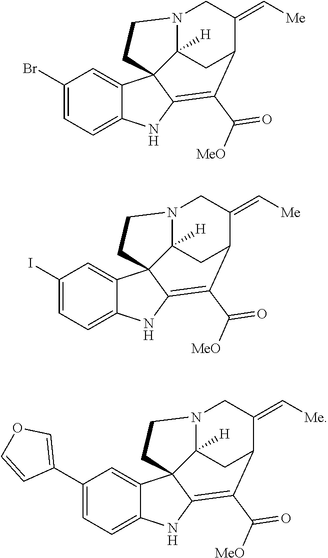

Aspect 78. A pharmaceutical composition comprising the compound of any one of Aspects 41-77 and a pharmaceutically acceptable carrier.

Aspect 79. A method of treating or preventing pain in a subject comprising administering to the subject a compound of any one of Aspects 41-77.

Aspect 80. The method of Aspect 79, wherein the subject has chronic pain.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure.

### Experimental

### Chemistry

General Experimental Procedures. All solvents and reagents were purchased from commercial sources and used directly without further purification. Akuamma seed powder was purchased from Relax Remedy and alkaloids were isolated and purified as previously described.⁴⁰ Bruker 400 MHz, Bruker 400 MHz HD, and Bruker 600 MHz spectrometers were used to record ¹H and ¹³C NMR spectra, and they were referenced to the residual solvent peaks (CHCl₃^{:1}H δ=7.26, ¹³C δ=77.16 ppm). High-performance liquid chromatography (HPLC) was conducted on an Agilent 1260 Infinity II fitted with a DAD detector and Phenomenox Luna Omega PS-C18 column (3 μm, 100×4.6 mm). Unless otherwise noted, acetonitrile and water each containing 0.1% formic acid with a flow rate of 1 mL/min were used as the mobile phase with following gradient: 20% MeCN for 1 min, increased to 45% MeCN over 10 min, decreased to 20% MeCN over 2 min, held at 20% MeCN for 1 min.

General procedure A: Akuammicine (1) (1.0 equiv) was placed in a clean, dry round-bottomed flask backfilled with nitrogen charged with a solution of 1:1 DCM/TFA (10 mL) on ice. NXS (1.1 eq) was dissolved in cooled 1:1 DCM/TFA (2 mL) and added dropwise to reaction mixture over 30 minutes while the reaction stirred at 0° C. After the addition of NXS, the reaction was allowed to stir at 0° C. for 3 h. Upon consumption of the starting material as indicated by TLC (9:1 DCM:MeOH), the reaction mixture was basified to pH 9-10 with 28% NH₄OH (aq). The aqueous layer was extracted with DCM (3×10 mL). The combined organic layers were dried with Na₂SO₄, filtered, and concentrated in vacuo. The crude residue was purified via silica gel chromatography (0-6% MeOH/DCM) to afford the product as an off-white solid.

General procedure B: To a clean, oven dried sealed tube was added Pd(PPh₃)₄ (0.05 equiv), RB(OH)₂ (2.0 equiv), oven dried K₂CO₃ (2.0 equiv), and 3 (1.0 equiv). The tube was sealed and purged with nitrogen. 2 mL of anhydrous toluene and anhydrous methanol (3:2) were degassed and injected into the tube. The reaction was heated to 80° C. for 24 h followed by cooling to room temperature and removal of solvent in vacuo. The crude residue was purified via silica gel chromatography (0-5% MeOH/DCM) to afford the product as a white solid.

General Procedure C: To a clean, oven-dried round bottom flask 1 or 2 (1 equiv) and NMO (3 equiv) was dissolved in a 3:2:1 solution of THF/tBuOH/H₂O (15 mL). The flask was purged with nitrogen and a catalytic amount of OSO₄ (4% wt. in H₂O, 0.1 mL) was added via syringe. The reaction was stirred for 12-18 h. The reaction was quenched with Na₂S₂O₃ (40% w.t. in H₂O) and extracted with DCM (4×30 mL). The combined organic layers were dried with Na₂SO₄, filtered, and concentrated under vacuum. The residue was purified using silica gel chromatography (6-10% MeOH in DCM, 5% NH₄OH by vol.) to give the final products as white solids.

General Procedure D: 1, 2, or 3 (1.0 equiv) were placed into a clean, dry round bottom flask and backfilled with nitrogen. The flask was charged with a 3:2 solution of AcOH/H₂O (5 mL) on ice. To the cooled solution was added NaCNBH₃ (1.1 equiv) and the reaction was stirred for 4 h. The reaction was diluted with DCM and basified with 28% NH₄OH (aq). The aqueous layer was extracted with DCM (4×10 mL). The combined organic layers were dried with Na₂SO₄, filtered, and concentrated in vacuo. The residue was purified using silica gel chromatography (0-4% MeOH in DCM) to give the final products as white solids.

General Procedure E: 1 or 2 (1.0 equiv) was placed in a clean, dry round-bottomed flask backfilled with nitrogen and dissolved in anhydrous chloroform (5 mL). Alkyl halide RI or RBr (200 equiv) was added to the flask via syringe at room temperature. The reaction was heated to 60° C. until consumption of the starting material as indicated by TLC (9:1 DCM:MeOH). The reaction mixture was cooled to room temperature and concentrated in vacuo. The residue was chromatographed over silica gel pre-treated with 12% NaBr in MeOH (4-10% MeOH in DCM) to yield the final products as white solids.

10-Bromoakuammicine (2). Prepared from 1 (120 mg, 0.371 mmol) according to General Procedure A to yield 161 mg (86%) of 2. ¹H NMR (400 MHz, CDCl₃) δ 8.98 (s, 1H), 7.37 (d, J=2.0 Hz, 1H), 7.28 (dd, J=8.3, 2.0 Hz, 1H), 6.71 (d, J=8.3 Hz, 1H), 5.47 (q, J=7.3 Hz, 1H), 4.21 (s, 1H), 4.02 (d, J=18.6 Hz, 2H), 3.81 (s, 3H), 3.46-3.40 (m, 1H), 3.16-3.04 (m, 2H), 2.57 (td, J=12.9, 6.9 Hz, 1H), 2.50-2.43 (m, 1H), 1.96 (dd, J=12.8, 5.9 Hz, 1H), 1.63 (dt, J=7.0, 1.8 Hz, 3H), 1.35 (dt, J=13.8, 2.9 Hz, 1H). ¹³C NMR (101 MHz, CDCl₃) δ 167.97, 166.99, 142.67, 139.02, 138.42, 130.70, 124.28, 121.39, 113.24, 110.91, 102.14, 62.00, 57.62, 56.76, 56.15, 51.28, 46.18, 30.90, 29.76, 13.02. HRMS calculated for C₂₀H₂₂N₂O₂Br: [M+H]⁺: 401.0868 (found); 401.0865 (calcd).

10-Iodoakuammicine (3). Prepared from 1 (120 mg, 0.371 mmol) according to General Procedure A to yield 167 mg (80%) of 3. ¹H NMR (400 MHz, CDCl₃) δ 8.98 (s, 1H), 7.52 (s, 1H), 7.46 (d, J=8.2 Hz, 1H), 6.61 (d, J=8.2 Hz, 1H), 5.43 (q, 1H), 4.13 (s, 1H), 3.97 (s, 2H), 3.81 (d, J=1.0 Hz, 3H), 3.36 (s, 1H), 3.13-3.00 (m, 2H), 2.55 (td, J=12.9, 6.8 Hz, 1H), 2.44 (d, J=13.8 Hz, 1H), 1.92 (s, 1H), 1.62 (d, J=6.9 Hz, 3H), 1.33 (d, J=13.6 Hz, 1H). ¹³C NMR (101 MHz, CDCl₃) δ 167.86, 166.30, 143.30, 139.05, 137.58, 136.89, 129.88, 122.24, 111.65, 102.23, 82.91, 62.02, 57.21, 56.65, 55.99, 51.33, 45.85, 30.71, 29.63, 13.09. HRMS calculated for C₂₀H₂₂N₂O₂I: [M+H]⁺: 449.0727 (found); 449.0726 (calcd).

10-Phenylakuammicine (4). Prepared from 3 (15 mg, 0.033 mmol) according to General Procedure B to yield 13 mg (73%) of 4. ¹H NMR (400 MHz, CDCl₃) δ 9.04 (s, 1H), 7.56-7.47 (m, 3H), 7.46-7.37 (m, 3H), 7.30 (t, J=7.6 Hz, 1H), 6.91 (d, J=8.2 Hz, 1H), 5.51 (q, J=7.2 Hz, 1H), 4.39 (s, 1H), 4.10 (d, J=14.9 Hz, 1H), 4.03 (s, 1H), 3.83 (s, 3H), 3.55 (td, J=12.6, 6.0 Hz, 1H), 3.21-3.11 (m, 2H), 2.62 (td, J=13.0, 6.8 Hz, 1H), 2.57-2.44 (m, 1H), 2.04 (dd, J=12.8, 5.3 Hz, 1H), 1.66 (d, J=7.0 Hz, 3H), 1.42 (dt, J=13.7, 2.9 Hz, 1H). ¹³C NMR (101 MHz, CDCl₃) δ 167.91, 167.16, 142.91, 141.11, 137.55, 137.08, 134.84, 128.97, 127.19, 126.98, 126.89, 122.61, 119.89, 109.88, 101.90, 62.05, 57.24, 56.71, 55.97, 51.29, 45.82, 30.74, 29.66, 13.16. HRMS calculated for C₂₆H₂₇N₂O₂: [M+H]⁺: 399.2074 (found); 399.2073 (calcd).

10-(3-Furanyl)akuammicine (5). Prepared from 3 (40 mg, 0.089 mmol) according to General Procedure B to yield 15.3 mg (44%) of 5. ¹H NMR (600 MHz, CDCl₃) δ 9.00 (s, 1H), 7.68 (t, J=1.2 Hz, 1H), 7.45 (t, J=1.7 Hz, 1H), 7.41 (d, J=1.6 Hz, 1H), 7.31 (dd, J=8.0, 1.7 Hz, 1H), 6.84 (d, J=8.1 Hz, 1H), 6.65 (dd, J=1.9, 0.9 Hz, 1H), 5.49 (q, J=7.3 Hz, 1H), 4.35 (s, 1H), 4.06 (d, J=14.9 Hz, 1H), 4.01 (s, 1H), 3.82 (s, 3H), 3.54 (s, 1H), 3.13 (m, J=6.4 Hz, 2H), 2.59 (td, J=12.9, 6.8 Hz, 1H), 2.48 (ddd, J=13.9, 4.1, 2.3 Hz, 1H), 2.01 (dd, J=12.8, 5.9 Hz, 1H), 1.65 (dt, J=7.0, 1.8 Hz, 3H), 1.39 (dt, J=14.0, 3.0 Hz, 1H), 0.88 (td, J=6.9, 1.8 Hz, 1H).¹³C NMR (151 MHz, CDCl₃) δ 167.74, 166.43, 143.85, 142.38, 137.97, 136.48, 136.24, 126.31, 126.12, 126.05, 124.05, 118.84, 110.04, 108.92, 101.86, 61.91, 56.51, 55.69, 51.36, 45.24, 30.43, 29.84, 29.41, 13.28. HRMS calculated for C₂₄H₂₅N₂O₃: [M+H]⁺: 389.1858 (found); 389.1865 (calcd).

10-(3-Thienyl)akuammicine (6). Prepared from 3 (40 mg, 0.089 mmol) according to General Procedure B to yield 16.0 mg (44%) of 6. Purity=36% (λ=254 nm) and 94% (λ=330 nm). ¹H NMR (600 MHz, CDCl₃) δ 9.02 (s, 1H), 7.54 (s, 1H), 7.41 (dd, J=8.1, 1.6 Hz, 1H), 7.37-7.32 (m, 3H), 6.85 (d, J=8.1 Hz, 1H), 5.46 (d, J=7.4 Hz, 1H), 4.31 (s, 1H), 4.03 (d, J=15.4 Hz, 1H), 3.99 (s, 1H), 3.82 (s, 3H), 3.51 (s, 1H), 3.13 (d, J=9.1 Hz, 1H), 3.11 (s, 1H), 2.58 (td, J=12.8, 6.7 Hz, 1H), 2.50-2.46 (m, 1H), 1.98 (dd, J=12.8, 5.8 Hz, 1H), 1.64 (dt, J=7.0, 1.7 Hz, 3H), 1.37 (d, J=13.8 Hz, 1H). ¹³C NMR (151 MHz, CDCl₃) δ 167.80, 166.71, 142.52, 142.15, 136.74, 135.87, 129.73, 126.57, 126.43, 126.30, 123.41, 119.40, 119.30, 109.89, 101.90, 61.85, 56.93, 56.49, 55.72, 51.33, 45.50, 30.54, 29.50, 13.23. HRMS calculated for C₂₄H₂₅N₂O₃S: [M+H]⁺: 405.1631 (found); 405.1637 (calcd).

10-Cyanoakuammicine (7). An oven dried, Schlenk tube was charged with 2 (30 mg, 0.075 mmol), Pd G3 tBuXPhos (3.6 mg, 0.004 mmol, 0.06 eq), and Zn(CN)₂ (6.6 mg, 0.056 mmol, 0.75 eq) and placed under an atmosphere of nitrogen. 2 mL of 1,4-dioxane and H₂O were added to the tube and the reaction was heated to 85° C. for 24 h. The reaction was cooled to room temperature and quenched with saturated NaHCO₃ (aq) and EtOAc, then stirred for 10 minutes. The reaction mixture was extracted with EtOAc (3×5 ml). The combined organic layers were dried with Na₂SO₄, filtered, and concentrated under vacuum. The crude residue was purified via silica gel chromatography (2-5% MeOH/DCM) to afford the product as an off-white solid (7.6 mg, 29% yield). ¹H NMR (400 MHz, CDCl₃) δ 9.22 (s, 1H), 7.51-7.44 (m, 2H), 6.85 (d, J=8.1 Hz, 1H), 5.41 (q, J=6.7 Hz, 1H), 4.09 (s, 1H), 3.99-3.88 (m, 2H), 3.82 (s, 3H), 3.37-3.25 (m, 1H), 3.08 (dd, J=12.6, 6.2 Hz, 1H), 3.00 (d, J=15.0 Hz, 1H), 2.53 (td, J=12.7, 6.8 Hz, 1H), 2.45 (ddd, J=13.7, 4.0, 2.3 Hz, 1H), 1.86 (dd, J=12.7, 5.0 Hz, 1H), 1.60 (dt, J=6.8, 1.8 Hz, 3H), 1.32 (dt, J=13.7, 3.2 Hz, 1H).¹³C NMR (101 MHz, CDCl₃) δ 167.76, 165.45, 147.30, 138.04, 137.73, 133.49, 124.49, 121.70, 119.78, 109.74, 104.25, 103.61, 62.11, 57.05, 56.72, 56.24, 51.50, 46.37, 30.80, 29.83, 13.04. HRMS calculated for C₂₁H₂₂N₃O₂: [M+H]⁺: 348.1710 (found); 348.1712 (calcd).

10-Nitroakuammicine (8). In an oven-dried round bottomed flask, 1 (40 mg, 0.12 mmol) was dissolved in anhydrous DCM. To the solution was added concentrated HNO₃ (420 mg, 6.7 mmol, 55 equiv). The reaction was allowed to stir for 1 h, then set in a cooling bath. The reaction was diluted with DI H₂O and saturated NaHCO₃ (aq) was added dropwise until basic. The aqueous solution was extracted with DCM (5×10 mL). The combined organic layers were dried with Na₂SO₄, filtered, and concentrated under vacuum. The crude sample was purified via silica gel chromatography (0-4% MeOH/DCM) to afford the product as a bright yellow solid (30 mg, 64% yield). ¹H NMR (400 MHz, CDCl₃) δ 9.36 (s, 1H), 8.16 (dd, J=8.6, 2.3 Hz, 1H), 8.11 (d, J=2.3 Hz, 1H), 6.86 (d, J=8.7 Hz, 1H), 5.40 (q, J=7.0 Hz, 1H), 4.11-4.05 (m, 1H), 3.98 (s, 1H), 3.92-3.82 (m, 4H), 3.31 (td, J=12.6, 5.7 Hz, 1H), 3.08 (dd, J=12.6, 6.6 Hz, 1H), 2.98 (d, J=15.1 Hz, 1H), 2.56 (td, J=12.6, 6.8 Hz, 1H), 2.46 (ddd, J=13.5, 4.0, 2.3 Hz, 1H), 1.87 (dd, J=12.5, 5.6 Hz, 1H), 1.62 (dt, J=6.9, 1.7 Hz, 3H), 1.34 (dt, J=13.6, 3.0 Hz, 1H). ¹³C NMR (101 MHz, CDCl₃) δ 167.74, 165.77, 149.19, 141.93, 138.36, 137.75, 125.75, 121.15, 117.14, 108.55, 105.20, 62.32, 57.17, 56.83, 56.43, 51.55, 46.57, 30.95, 30.00, 12.99. HRMS calculated for C₂₀H₂₂N₃O₄: [M+H]⁺: 368.1612 (found); 368.1610 (calcd).

10-Aminoakuammicine (9). In an oven-dried round bottom flask, 13 (35 mg, 0.095 mmol) and SnCl₂ (91 mg, 0.48 mmol, 5 eq) were dissolved in anhydrous EtOH. The flask was purged with nitrogen and heated to 70° C. for 4 h. The reaction was cooled to room temperature and concentrated under vacuum. The resulting residue was purified using silica gel chromatography (10-20% MeOH in DCM with 5% NH₄OH by vol.). The final product was obtained as a tan solid (17 mg, 52% yield). ¹H NMR (400 MHz, CDCl₃) δ 8.84 (s, 1H), 6.68 (d, J=2.2 Hz, 1H), 6.63 (d, J=8.1 Hz, 1H), 6.50 (dd, J=8.2, 2.2 Hz, 1H), 5.38 (q, J=7.1 Hz, 1H), 4.06 (s, 1H), 3.97-3.89 (m, 2H), 3.79 (s, 3H), 3.32 (ddd, J=12.6, 12.6, 5.8 Hz, 1H), 3.08-2.95 (m, 2H), 2.52 (ddd, J=12.7, 12.7, 6.8 Hz, 1H), 2.42 (ddd, J=13.6, 4.0, 2.3 Hz, 1H), 1.87 (dd, J=12.5, 5.7 Hz, 1H), 1.61 (dt, J=7.0, 1.7 Hz, 3H), 1.31 (dt, J=13.4, 3.0 Hz, 1H).¹³C NMR (101 MHz, CDCl₃) δ 168.53, 168.13, 140.89, 139.27, 138.44, 136.04, 120.90, 114.18, 110.08, 109.38, 100.12, 62.07, 57.98, 57.07, 56.33, 51.01, 46.27, 31.12, 29.84, 13.00. HRMS calculated for C₂₀H₂₄N₃O₂: [M+H]⁺: 338.1869 (found); 338.1869 (calcd).

19R,20R-Dihydroxyakuammicine (10) and 19S,20S-Dihydroxyakuammicine (11). Prepared from 1 (300 mg, 0.930 mmol) according to General Procedure C to afford 246 mg (74%) of 10 and 30.5 mg (9%) of 11. The ¹H and ¹³C NMR spectra of 10 were consistent with previously published spectral data.⁴⁵ Purity=93% as measured by HPLC at λ=254 nm and 330 nm. Compound 11: ¹H NMR (400 MHz, CDCl₃) δ 8.71 (s, 1H), 7.21-7.12 (m, 2H), 6.93 (t, J=7.4 Hz, 1H), 6.85 (d, J=7.8 Hz, 1H), 4.00 (q, J=6.4 Hz, 1H), 3.93 (s, 1H), 3.84 (s, 3H), 3.28 (d, J=12.3 Hz, 1H), 3.18-3.11 (m, 1H), 3.09 (d, J=4.1 Hz, 2H), 3.01-2.83 (m, 2H), 2.33 (d, J=12.3 Hz, 1H), 2.02 (dt, J=13.7, 3.3 Hz, 1H), 1.87 (ddd, J=13.0, 6.7, 1.9 Hz, 1H), 1.28 (d, J=6.4 Hz, 3H). ¹³C NMR (101 MHz, CDCl₃) δ 171.70, 169.26, 144.05, 135.48, 127.93, 121.62, 120.33, 109.94, 97.84, 72.80, 69.84, 59.77, 58.01, 54.30, 52.64, 51.84, 44.18, 35.65, 27.19, 16.82. HRMS calculated for C₂₀H₂₅N₂O₄: [M+H]⁺: 357.1808 (found); 357.1814 (calcd).

19-Hydroxyalstolucine (12). In a round bottomed flask, 10 (19.8 mg, 0.0555 mmol, 1 eq) was dissolved in DCM. Dess-Martin periodinane (47.5 mg, 0.122 mmol, 2 eq) and a drop of water were added. The flask was placed under nitrogen and allowed to stir at room temperature for 3-5 h. The reaction was quenched with saturated NaHCO₃(aq) and 40% wt. Na₂S2O₃ (aq) in a 1:1 ratio. The aqueous layer was extracted with DCM. The combined organic layers were washed with brine, dried with Na₂SO₄, filtered, and concentrated under vacuum. The residue was purified using silica gel chromatography (0-5% MeOH in DCM) to give the product as an off-white solid (10.2 mg, 54% yield). The ¹H NMR was consistent with previously published spectral data.^{45,76}

19,20-dihydroakuammicine (13). An oven-dried, round bottom flask was charged with 1 (50 mg, 0.19 mmol) and PtO₂ (6 mg, 10% by wt.) was added MeOH. The reaction was placed under an atmosphere of H₂ (g) and stirred for 8 h. Upon completion according to consumption of starting material by TLC, the reaction mixture was filtered through Celite and concentrated under vacuum. The residue was purified via silica gel flash chromatography (3-5% MeOH in DCM) to afford the final product as a white solid (53.2 mg, 88% yield). ¹H NMR (400 MHz, CDCl₃) δ 9.04 (s, 1H), 7.21 (d, J=7.4 Hz, 1H), 7.17 (td, J=7.7, 1.1 Hz, 1H), 6.93 (t, J=7.5 Hz, 1H), 6.84 (d, J=7.8 Hz, 1H), 4.21 (s, 1H), 3.77 (s, 3H), 3.43-3.12 (m, 3H), 3.08-2.84 (m, 2H), 2.32-2.12 (m, 2H), 2.01 (dd, J=13.1, 7.3 Hz, 1H), 1.95-1.82 (m, 1H), 1.48 (ddd, J=13.5, 4.0, 2.5 Hz, 1H), 1.40 (dt, J=13.3, 7.0 Hz, 1H), 1.14-0.97 (m, 4H). ¹³C NMR (101 MHz, CDCl₃) δ 170.16, 168.59, 144.37, 133.96, 128.38, 121.58, 119.90, 110.12, 98.76, 61.12, 55.20, 53.24, 51.34, 50.84, 41.58, 38.53, 30.69, 30.11, 25.89, 11.57. HRMS calculated for C₂₀H₂₅N₂O₂: [M+H]⁺: 325.1916 (found); 325.1916 (calcd).

2,16-dihydroakuammicine (14). Prepared from 1 (40.0 mg, 0.0892 mmol) according to General Procedure D to yield 78 mg (97%) of 14 without further purification. ¹H NMR (400 MHz, CDCl₃) δ 7.12-7.00 (m, 2H), 6.78 (td, J=7.4, 1.0 Hz, 1H), 6.61 (d, J=7.7 Hz, 1H), 5.29 (q, J=6.6 Hz, 1H), 4.15 (t, J=4.7 Hz, 1H), 4.07 (d, J=4.3 Hz, 1H), 3.79 (s, 3H), 3.60-3.52 (m, 2H), 3.24-3.20 (m, 1H), 3.20-3.10 (m, 2H), 2.96 (dt, J=10.8, 6.8 Hz, 1H), 2.70 (dd, J=5.4, 3.1 Hz, 1H), 2.41 (dt, J=13.5, 7.2 Hz, 1H), 2.22 (ddd, J=13.9, 3.9, 1.8 Hz, 1H), 2.10 (dt, J=12.9, 6.3 Hz, 1H), 1.71 (ddd, J=13.8, 4.5, 2.9 Hz, 1H), 1.62 (dd, J=6.9, 1.4 Hz, 3H). ¹³C NMR (101 MHz, CDCl₃) δ 174.72, 149.66, 140.87, 135.24, 128.02, 122.62, 119.75, 118.60, 110.02, 65.07, 64.35, 54.24, 54.04, 52.51, 52.33, 46.77, 38.59, 27.19, 23.17, 12.75. HRMS calculated for C₂₀H₂₅N₂O₂: [M+H]⁺: 325.1913 (found); 325.1916 (calcd).

2,16,19,20-tetrahydroakuammicine (15). To a round bottom flask with a 1:1 solution of AcOH/DI H₂O (2.5 mL) cooled to 0° C. was added 13 (18 mg, 0.056 mmol) and NaCNBH₃ (18 mg, 0.29 mmol, 5 eq). The reaction flask was purged with nitrogen and the reaction was stirred for 3 h. The reaction was diluted with CHCl₃ and basified with 28% NH₄OH (aq). The aqueous layer was extracted with CHCl₃ (4×5 ml). The combined organic layers were dried with Na₂SO₄, filtered, and concentrated under vacuum. The residue was purified via silica gel flash chromatography (3-5% MeOH in DCM) to afford the final product as a yellow solid (15 mg, 82% yield). ¹H NMR (400 MHz, CDCl₃) δ 7.10-6.98 (m, 2H), 6.77 (t, J=7.4 Hz, 1H), 6.59 (d, J=7.7 Hz, 1H), 4.20 (d, J=3.4 Hz, 2H), 3.78 (s, 3H), 3.16-3.03 (m, 1H), 3.02-2.84 (m, 3H), 2.60 (dt, J=19.6, 3.3 Hz, 2H), 2.35-2.15 (m, 4H), 1.74-1.64 (m, 2H), 1.32-1.20 (m, 1H), 1.17-1.06 (m, 1H), 0.95 (t, J=7.3 Hz, 3H).¹³C NMR (101 MHz, CDCl₃) δ 175.24, 149.26, 137.31, 127.80, 122.35, 119.77, 109.78, 67.05, 64.70, 55.63, 54.02, 52.30, 50.32, 42.67, 39.60, 39.00, 27.46, 26.15, 24.44, 11.67. HRMS calculated for C₂₀H₂₇N₂O₂: [M+H]⁺: 327.2075 (found); 327.2073 (calcd).

N-4-Methylakuammicine (16). Prepared from 1 (17 mg, 0.054 mmol) according to General Procedure E using methyl iodide (200 equiv) to afford 18 mg (65%) of 16. High-performance liquid chromatography (HPLC) was conducted on a Shimadzu LC-20AB fitted with a Shimadzu SPD-20A detector and Phenomenox Luna Omega PS-C18 column (3 μm, 100×4.6 mm). Acetonitrile and water each containing 0.1% formic acid with a flow rate of 1 mL/min were used as the mobile phase with following gradient: 5% MeCN for 5 min, increased to 20% MeCN over 7 min, increased to 45 MeCN over 5 min, increased to 95% over 3 min, held at 95% for 5 min, decreased to 5% MeCN over 2 min. ¹H NMR (400 MHz, CDCl₃) δ 8.82 (s, 1H), 8.03 (d, J=7.5 Hz, 1H), 7.20 (t, J=7.7 Hz, 1H), 7.01 (t, J=7.5 Hz, 1H), 6.84 (d, J=7.8 Hz, 1H), 5.86 (q, J=6.8 Hz, 1H), 4.90 (s, 1H), 4.54-4.32 (m, 3H), 4.23-4.10 (m, 2H), 3.89 (s, 3H), 3.80 (s, 3H), 2.97-2.84 (m, 1H), 2.52 (dt, J=14.9, 3.2 Hz, 1H), 2.25 (dd, J=14.2, 7.2 Hz, 1H), 1.78 (d, J=7.0 Hz, 3H), 1.68-1.57 (m, 1H). ¹³C NMR (101 MHz, CDCl₃) δ 166.95, 165.38, 143.24, 132.31, 129.43, 129.24, 128.16, 122.71, 122.65, 110.32, 101.43, 77.48, 77.16, 76.84, 72.82, 64.73, 63.35, 55.26, 51.75, 50.81, 40.92, 29.83, 28.28, 28.10, 13.40. HRMS calculated for C₂₁H₂₅N₂O₂: [M+H]⁺: 337.1922 (found); 337.1916 (calcd).

N-4-Allylakuammicine (17). Prepared from 1 (16 mg, 0.049 mmol) according to General Procedure E using allyl bromide (200 equiv) to afford 17 mg (83%) of 17. ¹H NMR (400 MHz, CDCl₃) δ 8.84 (s, 1H), 7.96 (d, J=7.5 Hz, 1H), 7.19 (m, 1H), 7.00 (td, J=7.6, 1.1 Hz, 1H), 6.84 (d, J=7.8 Hz, 1H), 6.21 (ddt, J=17.0, 9.7, 7.3 Hz, 1H), 6.00 (d, J=16.9 Hz, 1H), 5.80 (m, 2H), 5.16 (dd, J=12.8, 6.8 Hz, 1H), 5.05 (d, J=3.1 Hz, 1H), 4.50 (m, 2H), 4.21 (s, 1H), 4.14 (s, 1H), 3.94 (dd, J=11.9, 7.2 Hz, 1H), 3.81 (s, 3H), 2.87 (td, J=13.7, 7.3 Hz, 1H), 2.65 (dt, J=14.8, 3.2 Hz, 1H), 2.23 (dd, J=14.2, 7.3 Hz, 1H), 1.76 (d, J=7.0 Hz, 4H), 1.61 (s, 1H).¹³C NMR (101 MHz, CDCl₃) δ 166.97, 165.07, 143.08, 132.63, 130.47, 129.42, 129.29, 129.07, 125.39, 122.65, 122.59, 110.29, 101.53, 71.04, 63.58, 61.80, 60.36, 55.20, 51.72, 40.89, 28.72, 28.33, 13.51. HRMS calculated for C₂₃H₂₇N₂O₂: [M+H]⁺: 363.2068 (found); 363.2073 (calcd).

N-4-Propylakuammicine (18). Prepared from 1 (15 mg, 0.047 mmol) according to General Procedure E using 1-bromopropane (200 equiv) to afford 20 mg (90%) of 18. ¹H NMR (400 MHz, CDCl₃) δ 8.85 (s, 1H), 7.93 (d, J=7.5 Hz, 1H), 7.19 (d, J=7.6 Hz, 1H), 6.98 (t, J=7.5 Hz, 1H), 6.84 (d, J=7.8 Hz, 1H), 5.89 (q, J=6.9 Hz, 1H), 4.86-4.74 (m, 1H), 4.42 (d, J=14.2 Hz, 1H), 4.39-4.28 (m, 1H), 4.28-4.15 (m, 3H), 4.12 (s, 1H), 3.90 (td, J=12.0, 5.3 Hz, 1H), 3.80 (s, 3H), 2.89 (td, J=13.8, 7.3 Hz, 1H), 2.73 (dt, J=14.9, 3.2 Hz, 1H), 2.18 (dt, J=12.4, 6.2 Hz, 1H), 2.11-1.95 (m, 2H), 1.71 (d, J=6.9 Hz, 3H), 1.56 (d, J=14.9 Hz, 1H), 1.12 (t, J=7.2 Hz, 3H). ¹³C NMR (101 MHz, CDCl₃) δ 166.95, 164.47, 142.97, 132.87, 129.72, 129.65, 129.41, 122.52, 122.39, 110.28, 101.68, 77.48, 77.16, 76.84, 73.36, 65.03, 62.19, 61.11, 55.12, 51.66, 41.61, 28.85, 28.47, 17.72, 13.57, 11.05. HRMS calculated for C₂₃H₂₉N₂O₂: [M+H]⁺: 365.2223 (found); 365.2229 (calcd).

N-4-Cyclopropylmethylakuammicine (19). Prepared from 1 (16 mg, 0.085 mmol) according to General Procedure E using bromomethyl cyclopropyl (200 equiv) to afford 17 mg (77%) of 19. ¹H NMR (400 MHz, CDCl₃) δ 8.84 (s, 1H), 7.98 (d, J=7.5 Hz, 1H), 7.19 (t, J=7.7 Hz, 1H), 6.99 (t, J=7.5 Hz, 1H), 6.84 (d, J=7.8 Hz, 1H), 5.83 (q, J=6.9 Hz, 1H), 5.01 (d, J=3.2 Hz, 1H), 4.50 (td, J=12.6, 7.4 Hz, 1H), 4.40-4.28 (m, 3H), 4.21-4.09 (m, 2H), 3.89 (dd, J=13.2, 6.9 Hz, 1H), 3.80 (s, 3H), 2.91 (td, J=13.7, 7.2 Hz, 1H), 2.62 (dt, J=14.8, 3.2 Hz, 1H), 2.24 (dd, J=14.2, 7.2 Hz, 1H), 1.75 (d, J=7.0 Hz, 3H), 1.57 (dt, J=14.8, 2.9 Hz, 1H), 1.32-1.20 (m, 2H), 0.91-0.82 (m, 3H).¹³C NMR (101 MHz, CDCl₃) δ 166.97, 165.07, 143.08, 132.76, 129.38, 129.27, 129.15, 122.59, 122.53, 110.27, 101.53, 71.39, 65.91, 61.65, 60.67, 55.16, 51.69, 41.19, 28.74, 28.38, 13.49, 5.87, 5.20, 5.05. HRMS calculated for C₂₄H₂₉N₂O₂: [M+H]⁺: 377.2231 (found); 377.2229 (calcd).

N-4-Benzylakuammicine (20). Prepared from 1 (16 mg, 0.049 mmol) according to General Procedure E using benzyl bromide (200 equiv) to afford 21 mg (86%) of 20. ¹H NMR (400 MHz, CDCl₃) δ 8.81 (s, 1H), 7.88-7.81 (m, 2H), 7.53-7.42 (m, 3H), 7.24 (d, J=7.5 Hz, 1H), 7.14 (td, J=7.8, 1.1 Hz, 1H), 6.88-6.77 (m, 2H), 5.82-5.73 (m, 2H), 5.20 (d, J=12.6 Hz, 1H), 5.09 (d, J=3.2 Hz, 1H), 4.49 (td, J=12.5, 7.3 Hz, 1H), 4.34 (d, J=14.3 Hz, 1H), 4.26 (d, J=14.3 Hz, 1H), 4.16 (s, 1H), 3.89 (dd, J=11.8, 7.0 Hz, 1H), 3.80 (s, 3H), 2.91-2.71 (m, 2H), 2.15-2.05 (m, 1H), 1.75 (d, J=7.4 Hz, 3H), 1.58 (dd, J=11.0, 7.3 Hz, 1H). ¹³C NMR (101 MHz, CDCl₃) δ 166.99, 164.90, 142.96, 133.94, 132.80, 130.82, 129.69, 129.46, 129.26, 129.21, 128.14, 122.45, 122.18, 110.18, 101.62, 77.48, 77.16, 76.84, 69.63, 63.74, 62.08, 59.45, 55.04, 51.66, 40.73, 31.69, 29.81, 28.55, 28.45, 22.76, 14.22, 13.54. HRMS calculated for C₂₇H₂₉N₂O₂: [M+H]⁺: 413.2233 (found); 413.2229 (calcd).

10-Bromo-2,16-dihydroakuammicine (21). Prepared from 2 (28.3 mg, 0.0705 mmol) according to General Procedure D to yield 13.0 mg (46%) of 21. ¹H NMR (600 MHz, CDCl₃) δ 7.15 (d, J=2.0 Hz, 1H), 7.13 (dd, J=8.2, 2.0 Hz, 1H), 6.48 (d, J=8.2 Hz, 1H), 5.34 (q, J=7.6 Hz, 1H), 4.16-4.11 (m, 2H), 3.77 (s, 3H), 3.58 (d, J=14.7 Hz, 1H), 3.53 (t, J=3.4 Hz, 1H), 3.28-3.25 (m, 1H), 3.24-3.15 (m, 2H), 2.96 (dt, J=11.1, 6.9 Hz, 1H), 2.71 (dd, J=5.3, 3.1 Hz, 1H), 2.36 (dt, J=14.1, 7.3 Hz, 1H), 2.22 (ddd, J=14.0, 3.9, 1.9 Hz, 1H), 2.11 (dt, J=13.1, 6.3 Hz, 1H), 1.77-1.71 (m, 1H), 1.67-1.60 (m, 3H). ¹³C NMR (151 MHz, CDCl₃) δ 174.29, 148.61, 139.28, 137.08, 130.87, 125.75, 119.82, 111.40, 111.24, 64.74, 64.22, 54.20, 53.76, 52.45, 52.34, 46.40, 38.33, 27.11, 22.92, 12.84. HRMS calculated for C₂₀H₂₂N₂O₂Br: [M+H]⁺: 403.1019 (found); 403.1016 (calcd).

10-Iodo-2,16-dihydroakuammicine (22). Prepared from 3 (30.0 mg, 0.0669 mmol) according to General Procedure D to yield 11.3 mg (36%) of 22. ¹H NMR (600 MHz, CDCl₃) δ 7.34-7.29 (m, 2H), 6.40 (d, J=8.1 Hz, 1H), 5.35 (q, J=7.6 Hz, 1H), 4.16-4.11 (m, 2H), 3.77 (s, 3H), 3.60 (d, J=14.7 Hz, 1H), 3.53 (q, J=3.3 Hz, 1H), 3.30-3.26 (m, 1H), 3.25-3.21 (m, 1H), 3.20-3.15 (m, 1H), 2.96 (dt, J=11.1, 6.9 Hz, 1H), 2.71 (dd, J=5.4, 3.1 Hz, 1H), 2.35 (dt, J=14.2, 7.3 Hz, 1H), 2.22 (ddd, J=14.1, 3.9, 1.9 Hz, 1H), 2.11 (dt, J=13.1, 6.3 Hz, 1H), 1.75 (ddd, J=14.0, 4.5, 3.0 Hz, 1H), 1.63 (dt, J=6.9, 1.4 Hz, 3H). ¹³C NMR (151 MHz, CDCl₃) δ 174.23, 149.27, 138.97, 137.41, 136.87, 131.49, 120.09, 112.10, 80.43, 64.67, 64.02, 54.04, 53.70, 52.47, 52.30, 46.37, 38.27, 27.09, 22.85, 12.86. HRMS calculated for C₂₀H₂₄N₂O₂I: [M+H]⁺: 451.0887 (found); 451.0882 (calcd).

10-Bromo-N-4-methylakuammicine (23). Prepared from 2 (20.3 mg, 0.0506 mmol) according to General Procedure E using methyl iodide (200 equiv) to yield 29 mg (100%) of 23. ¹H NMR (600 MHz, CDCl₃) δ 8.83 (s, 1H), 8.31 (d, J=1.9 Hz, 1H), 7.29 (dd, J=8.3, 1.9 Hz, 1H), 6.73 (d, J=8.3 Hz, 1H), 5.87-5.81 (m, 1H), 5.04 (d, J=3.2 Hz, 1H), 4.51 (td, J=12.3, 7.4 Hz, 1H), 4.41 (dd, J=11.8, 7.4 Hz, 1H), 4.37 (d, J=14.3 Hz, 1H), 4.22 (d, J=14.2 Hz, 1H), 4.11 (d, J=3.4 Hz, 1H), 3.87 (s, 3H), 3.79 (s, 3H), 2.91 (td, J=13.7, 7.4 Hz, 1H), 2.51 (dt, J=14.8, 3.2 Hz, 1H), 2.26 (dd, J=14.5, 7.3 Hz, 1H), 1.76 (d, J=6.8 Hz, 3H), 1.62-1.56 (m, 1H). ¹³C NMR (151 MHz, CDCl₃) δ 166.89, 164.68, 142.44, 134.42, 132.20, 129.06, 127.89, 125.86, 114.54, 111.67, 102.09, 72.13, 64.27, 63.04, 55.17, 51.83, 50.12, 40.85, 28.18, 28.04, 13.35. HRMS calculated for C₂₁H₂₁N₂O₂Br: [M+H]⁺: 415.1021 (found); 415.1021 (calcd).

10-Bromo-N-4-allylakuammicine (24). Prepared from 2 (25.0 mg, 0.0623 mmol) according to General Procedure E using allyl bromide (200 equiv) to yield 31.3 mg (96%) of 24. ¹H NMR (600 MHz, CDCl₃) δ 8.87 (s, 1H), 8.12 (d, J=1.9 Hz, 1H), 7.30 (dd, J=8.3, 1.9 Hz, 1H), 6.75 (d, J=8.3 Hz, 1H), 6.22 (ddt, J=17.1, 10.0, 7.3 Hz, 1H), 6.03-5.97 (m, 1H), 5.79 (dt, J=4.0, 2.1 Hz, 1H), 5.12 (dd, J=12.8, 6.9 Hz, 1H), 5.04 (q, J=2.4 Hz, 1H), 4.59 (dd, J=12.7, 7.8 Hz, 1H), 4.41 (td, J=12.4, 7.4 Hz, 1H), 4.26 (q, J=14.3 Hz, 2H), 4.13 (s, 1H), 4.05 (dd, J=11.9, 7.2 Hz, 1H), 3.81 (s, 3H), 2.89 (td, J=13.7, 7.3 Hz, 1H), 2.72 (dt, J=15.0, 3.2 Hz, 1H), 2.24 (dd, J=14.3, 7.2 Hz, 1H), 1.76-1.72 (m, 3H), 1.56 (d, J=14.9 Hz, 1H). ¹³C NMR (151 MHz, CDCl₃) δ 166.82, 163.93, 142.22, 134.58, 132.27, 130.52, 129.48, 128.84, 125.62, 125.32, 114.43, 111.70, 102.31, 70.71, 63.64, 61.95, 60.30, 55.06, 51.80, 40.93, 28.69, 28.21, 13.49. HRMS calculated for C₂₃H₂₆N₂O₂Br: [M+H]⁺: 441.1180 (found); 441.1178 (calcd).

10-Bromo-N-4-cyclopropylmethylakuammicine (25). Prepared from 2 (25.0 mg, 0.0623 mmol) according to General Procedure E using bromomethyl cyclopropane (25 equiv) to yield 10.4 mg (31%) of 25. ¹H NMR (600 MHz, CDCl₃) δ 8.14 (d, J=2.0 Hz, 1H), 7.32 (dd, J=8.3, 1.9 Hz, 1H), 6.75 (d, J=8.3 Hz, 1H), 5.85 (q, J=7.3 Hz, 1H), 5.05 (s, 1H), 4.46 (td, J=12.4, 7.4 Hz, 1H), 4.36 (d, J=5.7 Hz, 2H), 4.32 (dd, J=12.9, 7.2 Hz, 1H), 4.24-4.20 (m, 1H), 4.14 (s, 1H), 3.91 (dd, J=13.1, 6.9 Hz, 1H), 3.81 (s, 3H), 2.93 (td, J=13.8, 7.2 Hz, 1H), 2.65 (dt, J=15.0, 3.2 Hz, 1H), 2.27 (dd, J=14.3, 7.2 Hz, 1H), 1.75 (d, J=6.9 Hz, 3H), 1.57 (d, J=14.9 Hz, 1H), 1.28-1.24 (m, 1H), 0.88 (t, J=6.4 Hz, 3H). ¹³C NMR (151 MHz, CDCl₃) δ 166.88, 164.12, 142.28, 134.68, 132.34, 129.43, 128.88, 125.63, 114.53, 111.75, 102.33, 71.16, 65.91, 61.63, 60.59, 55.09, 51.85, 41.16, 28.72, 28.31, 13.53, 5.88, 5.30, 5.09. HRMS calculated for C₂₄H₂₈N₂O₂Br: [M+H]⁺: 455.1331 (found); 455.1334 (calcd).

10-Bromo-19R,20R-dihydroxyakuammicine (26). Prepared from 2 (55.3 mg, 0.138 mmol) according to General Procedure C to yield 27.6 mg (46%) of 26. ¹H NMR (600 MHz, CDCl₃) δ 9.08 (s, 1H), 7.28 (d, J=2.0 Hz, 1H), 7.23 (dd, J=8.2, 2.0 Hz, 1H), 6.68 (d, J=8.2 Hz, 1H), 3.81 (d, J=3.2 Hz, 1H), 3.76 (s, 3H), 3.56 (q, J=6.2 Hz, 1H), 3.10-3.03 (m, 2H), 2.97 (t, J=2.6 Hz, 1H), 2.93-2.82 (m, 2H), 2.71 (dt, J=13.4, 3.2 Hz, 1H), 2.45 (d, J=13.0 Hz, 1H), 1.87 (dd, J=13.4, 6.7 Hz, 1H), 1.39 (d, J=6.2 Hz, 3H), 1.13 (dt, J=13.2, 3.0 Hz, 1H). ¹³C NMR (151 MHz, CDCl₃) δ 171.06, 168.25, 143.25, 137.79, 130.49, 123.28, 113.45, 111.21, 101.42, 73.52, 72.79, 60.38, 56.76, 54.10, 52.61, 51.34, 42.97, 34.31, 26.24, 17.76. HRMS calculated for C₂₀H₂₄N₂O₄Br: [M+H]⁺: 435.0924 (found); 435.0919 (calcd).

10-Bromo-19,20-dihydroakuammicine (27). Prepared from 13 (20.2 mg, 0.062 mmol) using General Procedure A to yield 8.6 mg (42%) of 27. ¹H NMR (600 MHz, CDCl₃) δ 9.03 (s, 1H), 7.25 (d, J=2.0 Hz, 1H), 7.22 (dd, J=8.2, 1.9 Hz, 1H), 6.67 (d, J=8.2 Hz, 1H), 3.86 (d, J=3.8 Hz, 1H), 3.75 (s, 3H), 3.17-3.13 (m, 1H), 3.10-3.03 (m, 1H), 2.97-2.90 (m, 2H), 2.85 (dd, J=11.6, 7.2 Hz, 1H), 2.09-1.96 (m, 2H), 1.85 (dd, J=13.4, 6.7 Hz, 1H), 1.70 (s, 1H), 1.40-1.33 (m, 2H), 1.06-0.98 (m, 1H), 0.97 (t, J=7.1 Hz, 3H). ¹³C NMR (151 MHz, CDCl₃) δ 170.51, 168.94, 143.65, 137.84, 130.41, 123.23, 113.23, 111.12, 99.66, 61.27, 56.50, 54.15, 51.41, 51.28, 43.15, 39.40, 31.53, 31.04, 26.24, 11.82. HRMS calculated for C₂₀H₂₄N₂O₂Br: [M+H]⁺: 405.0997 (found); 405.0995 (calcd).

### In Vitro PHARMACOLOGY

Drugs. Dimethyl sulfoxide (DMSO), forskolin, [D-Ala2, N-Me-Phe4, Gly5-ol]-Enkephalin acetate salt (DAMGO), and trans-(±)-3,4-dichloro-n-methyl-N-(2-(1 pyrrolidinyl cyclohexyl)benzeneacetamide methanesulfonate (U50,488) were purchased from Sigma-Aldrich (St. Louis, MO, United States). [³H]DAMGO (53.7 Ci/mmol, lot #2376538; 51.7 Ci/mmol, lot #2815607) and [3H]U69,593 (60 Ci/mmol, lot #2367921 and lot #2644168; 49.2 Ci/mmol, lot #2791786) were purchased from PerkinElmer (Waltham, MA, United States).

Cell Lines and Cell Culture. To perform the HitHunter cAMP accumulation assay, Chinese hamster ovary cells (CHO-K1) stably expressing human μ-opioid receptor (OPRM1, catalog no. 95-0107C2, DiscoverX, Fremont, CA, USA), K-opioid receptor (OPRK1, catalog no. 95-0088C2, DiscoverX, Fremont, CA, USA), or 6-opioid receptor (OPRD1, catalog no. 95-0108C2, DiscoverX, Fremont, CA, USA) were maintained at 80% confluence in F-12 media supplemented with 10% fetal bovine serum, 1% penicillin/streptomycin/L-glutamine, and 800 μg/mL G418. To perform the PathHunter β-arrestin-2 recruitment assay, U2OS-human κ opioid receptor (κOR) cells stably expressing the κOR and β-arrestin-2 (RRID:CVCL_LA97, DiscoverX, Fremont, CA, USA) were maintained in McCoy's 5A media supplemented with 10% FBS and containing 500 μg/mL geneticin and 250 μg/mL hygromycin. All cells were grown 37° C. under 5% CO₂ in a humidified incubator under sterile conditions in T75 flasks. During passaging, cells were dislodged from the flask following a 3-5 min incubation with 0.25% trypsin or non-enzymatic detachment reagent and subcultivated at ratios of 1:10 (CHO) and 3:10 (U2OS).

HitHunter cAMP Accumulation Assay. CHO-K1 OPRM1, OPRK1, and OPRD1 cells (DiscoverX, Fremont, CA, USA) were maintained with F-12 media until 70-80% confluent. When confluent, cells were plated at a concentration of 10,000 cells/well in Assay Complete Plating Reagent (Eurofins) on a 384-well white tissue culture plate. The cells were incubated at 37° C. under 5% CO₂ in a humidified incubator for 24 hours. Stock solutions (5 or 10 mM) of all ligands were created in DMSO and used to serially dilute in 100% DMSO (100×). Assay buffer (HBSS+10 mM HEPES) was used to dilute the 100×DMSO dilutions to 10×. Assay buffer with forskolin (200 μM) was used to dilute the 10× dilutions to a 5× working concentrations. The HitHunter cAMP Assay for Small Molecules (Eurofins) was used according to manufacturer's instructions to measure the cAMP inhibition of the serial dilutions in triplicate. Luminescence was quantified using a FlexStation 3 (Molecular Devices) or a Cytation 5 (BioTek) microplate reader.

PathHunter β-arrestin-2 Recruitment Assay. The R-arrestin-2 recruitment assay was performed using U2OS-human (κOR) PathHunter R-arrestin-2 cells (DiscoverX) as previously described.⁷⁷

Competitive Radioligand Binding. Cell membranes were isolated from CHO and U2OS cells which stably express the μOR and the κOR, respectfully (DiscoverX). The competitive radioligand binding assay was performed on the membrane preparations as previously described with using tritiated radioligands [³H]DAMGO and [³H]U69,593 for the μOR and the κOR, respectfully.⁷⁸

Statistics. Cellular pharmacological data was analyzed using GraphPad 9 (GraphPad Prism software, La Jolla, CA, United States) and is presented as mean±SEM. Composite figures are representative curves averaged from a minimum of three independent assays which were normalized to vehicle, forskolin, and ligand controls.

### Computational

Structure alignment and refinement. The LigPrep feature in Maestro (Schrödinger Suite, 2023-1) was used to compute possible conformation of 5 at pH=7.2 using the OPLS4 force field. The conformation of 5 bearing a protonated nitrogen was aligned to MOM-SalB bound to the κOR (PDB: 8DZP), which had previously been prepared using the Protein Preparation feature. Using the superposition feature, the furan ring and ester of 5 were overlayed onto the furan ring and ester of MOM-SalB. A ligand-receptor complex was formed by replacing MOM-SalB with 5, and the resulting complex was refined with Prime using the VSGB solvation model, OPLS4 force field, and hierarchal sampling algorithm. Atoms within 5.0 Å of the ligand were refined.

Molecular dynamics simulation. The ligand-protein complex resulting from the Prime refinement was_uploaded to CHARMM-GUI web server⁷⁹ to prepare necessary files for running MD simulation in NAMD (Version 3.0b6).⁸⁰ In CHARMM-GUI web server, the ligand-protein complex was placed in a rectangular box and hydrated using TIP3P water model. Then the simulation box was neutralized using counter ions (0.15 M NaCl). Periodic boundary conditions were imposed during the course of the simulation. Electrostatic interactions were computed with the particle mesh Ewald algorithm, and Lennard-Jones interactions were truncated at 12 Å. The CHARMM36m force field⁸¹ was used for the protein atoms, while ligand parameter files were prepared using the CHARMM General force field (CgenFF).⁸² A minimization was done for 20 ps and followed by MD simulation using isothermal-isochoric ensemble (NVT) for 1 ns to relax the system. During this step, Harmonic restraint parameters were applied to protein and ligand heavy atoms to relax only waters and ions. Further system relaxation was achieved using the NPT ensemble for another 1 ns.^{83,84} Finally, the system was subjected to 100 ns MD simulation using the NPT ensemble. For all calculations, the time step parameter was set to 2 femtoseconds. The NVT ensemble was maintained at 310 K and the NPT ensemble at 310 K and 1.01325 bar. VMD software⁸⁵ was used to extract frames from the simulation trajectories, align protein structures, and calculate RMSD values for protein and ligand in each frame. Figures were prepared with PyMol.

### Results

### Chemistry

To begin our SAR studies, we recognized that 1 contained four apparent functional groups capable of making ligand-receptor interactions with the κOR: (1) the aromatic core, (2) the external olefin appended at C20, (3) the vinylogous carbamate, and (4) the tertiary nitrogen. To investigate the nature of these potential interactions, a series of highly chemoselective reactions were employed to modify these sites on 1. To begin these studies 1 was isolated from commercial P. nitida seeds and purified using a pH-zone refining counter-current chromatography as previously described.⁴⁰

### C10 Modifications

The modification of the aromatic core began with the acid-mediated halogenation of 1 with N-bromo succinimide (NBS) and N-iodo succinimide (NIS),⁴² which exclusively produced 10-bromo akuammicine (2) and 10-iodo akuammicine (3) (FIG. 1). In addition to introducing additional steric bulk, lipophilicity, and potential for halogen-bonding interactions, these aryl halides also serve as excellent functional handles to install substitutions to probe the κOR binding pocket. For instance, furan rings exist in numerous κOR ligands, including nalfurafine, salvinorin A, and triazole 1.1, suggesting this heterocycle may represent a privileged scaffold for the κOR. Fortunately, 3 demonstrated to be a competent coupling partner with aryl and heterocyclic boronic acids to produce analogues 4-6.43

Further C10 modifications were pursued to probe the effect of appending electron-withdrawing and electron-donating groups on the aromatic ring. Palladium-catalyzed cyanation⁴⁴ afforded the 10-cyanoakuammicine (7). Additionally, nitration of 1 exclusively afforded the 10-nitroakuammicine (8), further illustrating a strong preference for electrophilic aromatic substitution to occur at the C10 position of 1. Reduction of 8 with stannous chloride produced the corresponding aryl amine 9. Although 9 is a useful counterpoint to the electron-poor rings in 7 and 8, and introduces potential hydrogen-bonding interactions, the compound could not be isolated with sufficient purity despite considerable efforts to purify it via normal and reverse-phase chromatography. Furthermore, the low yields of this reduction and difficulties in its purification prevented its use as a synthetic intermediate for further modification.

### C2,16 and C19,20 Olefin Modifications

Alstolucines A-F are a small family of alkaloids isolated from Alstonia spatulata that bear the same pentacyclic framework as 1, but with oxidized side chains in place of the C19,20 olefin.⁴⁵ Despite this structural similarity to 1, the pharmacological activity of the alstolucines at the κOR has not been assessed. To explore how additional polarity at this position would influence opioid activity, we employed a similar route originally developed by the Andrade group to oxidize the external olefin of 1.45 Upjohn dihydroxylation of the C19,20 olefin of 1 yielded the diastereomeric syn-diol products 10 and 11 in a 9:1 ratio with the major product, 10, arising from the addition of osmium tetroxide to the convex side of 1 (Scheme 2).⁴⁵ Notably, 11 was not originally reported by Andrade, likely due to fact it is produced in low yield. Swern oxidation of the secondary alcohol in 10 produced the ketone 12.45

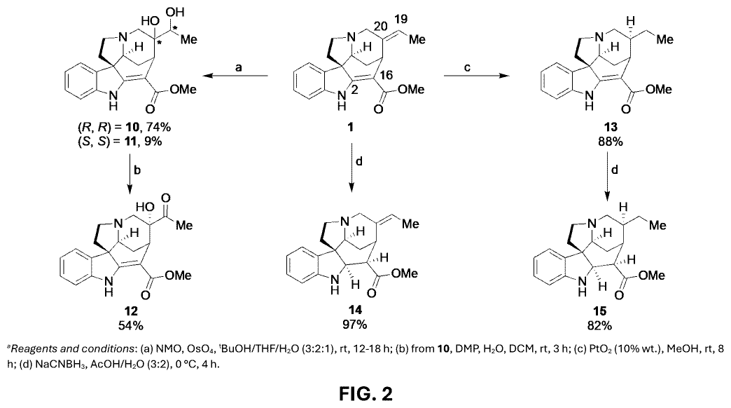

In addition to introducing hydrogen bond donors and acceptors, dihydroxylation of the exocyclic olefin in 1 also introduces a stereogenic center at C19 and flexibility in the C19-C20 bond. In an attempt to separate steric effects from the polar effects of the oxygens in 10-12, we also reduced the olefin with platinum (IV) oxide to yield 19,20-dihydroakuammicine (13).⁴⁵⁻⁴⁷ As noted by Andrade, this reduction is both stereoselective and chemoselective, producing 13 as a single diastereomer and no apparent reduction of the vinylogous carbamate. Exposure of 1 or 13 to sodium cyanoborohydride⁴⁶ selectively reduced the C2,16 internal olefin to produce the 2,16-dihydroakuammicine (14) and 2,16,19,20-tetrahydroakuammicine (15), respectively (FIG. 2).

### N4 Alkylation

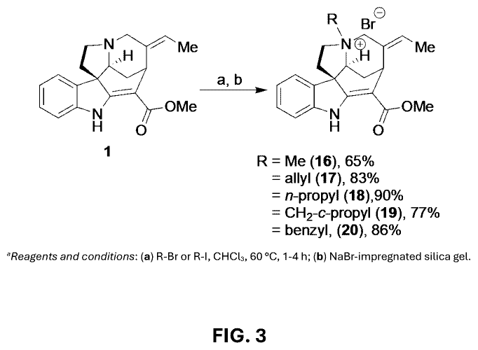

On the morphinan scaffold μOR antagonists such as naloxone and naltrexone typically feature allyl and cyclopropyl methyl appendages on the tertiary nitrogen, whereas agonists such as morphine and oxycodone have a methyl group at this position.^{48,49} Additionally, methylation of the tertiary amine, for example as seen in gut motility agent methylnaltrexone, has proven to be an effective strategy for peripherally restricting morphine-derived opioids while maintaining affinity for the receptor.⁵⁰ To see whether similar trends apply to 1, the tertiary amine was alkylated with the corresponding alkyl halide to introduce a methyl, propyl, allyl, cyclopropylmethyl, and benzyl group (FIG. 3). Because 16-20 bear quaternary amines, their purification using normal phase silica gel chromatography proved challenging. Instead, a modified ion exchange column using silica gel presaturated with a methanolic solution of sodium bromide was used to purify the alkyl derivatives as bromide salts.⁵¹

### Doubly Substituted Derivatives

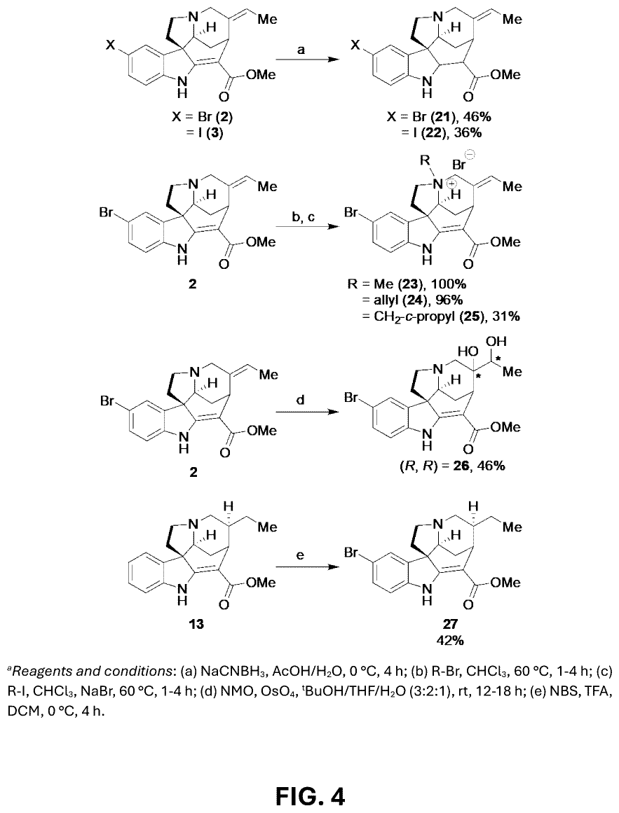

An initial pharmacological characterization of the C10-substituted, olefin-modified, and N-alkylated derivatives revealed that C10 halogenation led to significant increases in κOR potency (vide infra). Based on this observation, we also prepared a series of halogenated derivatives that contain modifications to the olefins or tertiary amine (21-27, FIG. 4).

Employing similar conditions to those described above to modify 1, reduction of the C2,16 olefin of halogenated analogues 2 and 3 selectively produced analogues 21 and 22 as single diastereomers. The N-alkylation procedure was similarly adapted to yield analogues 23-25 from 2. However, the Upjohn dihydroxylation of 2 produced considerably lower yields compared to that of 1. As a result, only the major product (26) could be isolated in sufficient amounts for evaluation. Finally, concerned that the reduction of the C19,20 olefin with PtO₂ would also result in protodehalogenation, we opted to brominate 13 to synthesize 27.

### In Vitro Pharmacology

Having generated a collection of akuammicine analogues with modifications to the C10 (2-8), C19,20 (10-13, 15, 26, 27), C2,16 (14, 21, 22) and N4 (16-20, 23-25) positions, we looked to examine how these modifications would impact opioid receptor activity. Because our previous studies indicated the akuamma alkaloids are moderately potent κOR and μOR agonists with negligible activity at the δOR,^{40,41} all compounds were first evaluated at the κOR and μOR using a cell-based system that measured inhibition of forskolin-induced production of cyclic adenosine monophosphate (cAMP) as a measure of G-protein pathway activation (Table 1). Consistent with our initial characterization of 1 as a selective κOR agonists, except in instances where the modifications removed all agonist activity, the derivatives were full κOR agonists with considerably greater potency at the κOR than the μOR. And while most of these derivatives possess similar efficacies, it was noted that several modifications had considerable effects on the compounds' potency at the κOR.

TABLE 1
Inhibition of cAMP by analogues of 1 at the κOR and the μOR

| | κOR cAMP inhibition^{a} | | | μOR cAMP inhibition^{a} | | | |
|---|---|---|---|---|---|---|---|
| | pEC₅₀ ± | EC₅₀ | Eₘₐₓ ± SEM | pEC₅₀ ± | EC₅₀ | Eₘₐₓ ± SEM | |
| Compound | SEM^{b} | (nM) | (%)^{b} | SEM^{b} | (nM) | (%)^{b} | Selectivity^{c} |
| U50,488 | 9.0 ± 0.1 | 0.98 | 100 ± 2 | — | — | — | |
| DAMGO | — | — | — | 9.2 ± 0.1 | 0.6 | 100 ± 2 | |
| 1 | 5.9 ± 0.1 | 1,200 | 91 ± 7 | — | — | <10% | — |
| 2 | 8.4 ± 0.1 | 3.9 | 96 ± 2 | 5.0 ± 0.9 | 11,000 | 62 ± 64 | 2,800 |
| 3 | 8.2 ± 0.1 | 5.7 | 98 ± 2 | 4.4 ± 1.9 | 41,000 | <10% | 7,200 |
| 4 | 7.8 ± 0.1 | 15 | 101 ± 2 | 6.1 ± 0.1 | 780 | 106 ± 6 | 52 |
| 5 | 9.1 ± 0.1 | 0.88 | 102 ± 2 | 6.1 ± 0.2 | 720 | 99 ± 12 | 820 |
| 6 | 8.1 ± 0.1 | 7.7 | 104 ± 3 | 6.1 ± 0.1 | 840 | 107 ± 3 | 110 |
| 7 | 7.1 ± 0.2 | 81 | 102 ± 6 | — | — | <10% | — |
| 8 | 6.8 ± 0.1 | 160 | 96 ± 5 | 5.4 ± 0.8 | 4,000 | <10% | 25 |
| 10 | — | — | <10% | — | — | <10% | — |
| 12 | — | — | <10% | — | — | <10% | — |
| 13 | 5.8 ± 0.1 | 1,600 | 99 ± 9 | — | — | <10% | — |
| 14 | — | — | <10% | — | — | <10% | — |
| 15 | — | — | <10% | — | — | <10% | — |
| 16 | — | — | <10% | — | — | <10% | — |
| 17 | — | — | <10% | — | — | <10% | — |
| 18 | — | — | <10% | — | — | <10% | — |
| 19 | 5.3 ± 0.3 | 4,700 | 113 ± 24 | — | — | <10% | — |
| 20 | — | — | <10% | 5.6 ± 0.1 | 2,300 | 112 ± 6 | — |
| 21 | 7.3 ± 0.1 | 47 | 100 ± 4 | — | — | <10% | — |
| 22 | 7.6 ± 0.1 | 24 | 100 ± 5 | — | — | <10% | — |
| 23 | 6.0 ± 0.1 | 940 | 102 ± 7 | — | — | <10% | — |
| 24 | 6.2 ± 0.1 | 650 | 100 ± 7 | — | — | <10% | — |
| 25 | 6.7 ± 0.1 | 210 | 94 ± 5 | — | — | <10% | — |
| 26 | — | — | <10% | — | — | <10% | — |
| 27 | 7.5 ± 0.1 | 31 | 100 ± 5 | 6.0 ± 0.1 | 1,100 | 107 ± 5 | 35 |

^{a}cAMP inhibition at the κOR and the μOR was determined through the HitHunter assay.
^{b}Mean ± standard error on the mean from a minimum of three independent assays.
— = no activity observed at 10 μM.
^{c}Selectivity index = μOR EC₅₀/κOR EC₅₀.

### C10 Modifications

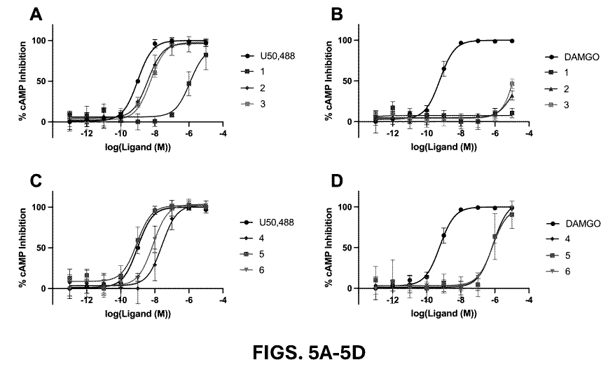

The most dramatic increase in κOR potency was observed in compounds bearing substitutions to the C10 position of the aromatic ring. Compounds 2 and 3 with a bromine and iodine at this position are potent κOR agonist (EC₅₀=3.9 and 5.7 nM), representing a >300- and 200-fold increase in potency relative to 1 (FIG. 5A, Table 1). Despite this striking increase in potency elicited by a single atom substitution, this effect appears to be specific to the κOR and 2 and 3 only weakly activated the μOR thereby maintaining >2,800-fold selectively for the κOR over the μOR (FIG. 5B, Table 1). Similar trends were also observed when the affinity of 2 and 3 were measured at the κOR and μOR using a radioligand displacement assay. Compounds 2 and 3 tightly bind to the κOR (Kᵢ=0.36 nM and 0.087 nM, respectively) and have 15,000- and 40,000-fold greater affinity for the κOR over the μOR (Kᵢ=5,500 nM and 3,600 nM, respectively).

Compounds 1-6 were evaluated using the HitHunter assay that measures inhibition of forskolin-induced cAMP at the κOR (A and C) μOR (B and D). All curves are representative of the averaged values from a minimum of three independent assays.

Functionalization of the C10 position of 1 with phenyl (4), 3′-furanyl (5), and 3′-theinyl (6) moieties also elicited a significant effect on κOR and μOR potency (FIG. 5C,D). Analogue 4 activated the κOR more potently than 1 (EC₅₀=15 nM). Likewise, compound 5 activated the κOR >1,300 times more potently (EC₅₀=0.88 nM) than the parent compound 1. Notably, 5 was the most potent analogue identified and possesses similar potency as the control agonist U50,488 (Table 1). Moreover, analogue 6 also potently activates the κOR (EC₅₀=7.7 nM). Unlike the halogenated derivatives, compounds 4-6 do activate the μOR (EC₅₀ 720-840 nM) and thus have less selectivity for the κOR than 2 and 3.

Installation of the nitrile (7) to the C10 position also modulated κOR activity (EC₅₀=81 nM) without activating the μOR. A similar effect as 7 on κOR potency was observed with the C10 nitro analogue (8), most likely due to their similar electron-withdrawing capabilities. However, compared to 7, analogue 8 has slightly reduced κOR potency (EC₅₀=160 nM) and does weakly activate the μOR (EC₅₀=4,000 nM) (FIG. 5C-D).

### Olefin Oxidation and Reduction

While substitutions at C10 appear to improve κOR potency, changes to the olefins were generally less well-tolerated (Table 1). Compounds 10 and 12 bearing olefin oxidations possess no agonist activity at either the κOR or μOR. Conversely, reduction of the C19,20 olefin (13) did not have a considerable impact on κOR potency (EC₅₀=1,600 nM), producing a similar agonist effect as the natural product 1. Because the ethyl group of 13 appears to be tolerated, the reduced activity of 10 and 12 is likely due to the additional polarity introduced by the oxygens, rather than forcing the ethyl group into an unfavorable position within the κOR binding site. As opposed to the external olefin, when the C2,16 olefin is reduced (14 and 15) all κOR agonist activity is abolished or significantly diminished. This might suggest that the vinylogous carbamate is required for κOR activation and perhaps even serving as an electrophilic site to covalently bind to the κOR. However, analogues 21 and 22 bearing halogens at the C10 of 15 do have κOR activity (EC₅₀=47 and 24 nM, respectively). Instead, it is more likely that breaking the extended system of conjugation between the ester and the arylamine forces the ester into an orientation that makes less favorable interactions with the κOR. Interestingly, halogenation of the C10 position is not sufficient to rescue the activity of 10 (26), which further supports the notion that the exocyclic olefin most likes points into a small hydrophobic pocket where small, non-polar functionalities are not tolerated.

### N4 Alkylation

Generally, alkylation of the tertiary nitrogen of 1 was detrimental to opioid activity. The alkylated analogues 16-18 and 20 have no agonistic activity at the κOR or μOR; however, the cyclopropylmethyl analogue 19, did produce κOR agonistic activity (EC₅₀=4,700 nM) albeit with about 4-fold reduced potency compared to 1. Introduction of a bromine atom at C10 once again rescues the agonist activity of the N4-alkylated analogues (23-25). While these alkylated analogues are considerably less potent than 2 (EC₅₀=210-940 nM), they did surpass the potency of the parent natural product 1. Additionally, the potency of the C10 brominated, N4 alkylated analogues increased as the steric bulk of the alkylation increased (cPr>allyl>Me), suggesting the N4 alkyl substituents may be reaching into a hydrophobic pocket of the κOR.

### Additional In Vitro Characterization of Derivatives

Our initial characterization of the akuamma alkaloids indicated that 1 had low affinity and little agonist activity at the δOR.⁴⁰ To determine whether this was also true of the derivatives of 1, several of the more potent agonists (2-8 and 22) were evaluated alongside 1 for their ability to activate the δOR. Consistent with our previous results, none of the compounds produced agonist activity at concentrations <1 μM. This indicates that the akuammicine scaffold imparts a stronger bias against δOR activation.

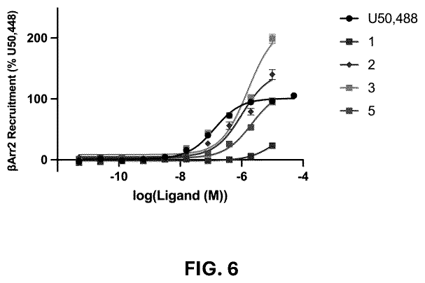

Given the striking increase in potency at the κOR in the HitHunter cAMP inhibition assay produced by C10 substitutions, the opioid receptor signaling of 1-8 and 22 were further examined. As discussed, several early studies have hypothesized that G-protein biased κOR agonists are less aversive and sedative than balanced agonists.^{12,52,53} Therefore, we first evaluated the ability of 1-8 and 22 to induce βArr2 recruitment using the PathHunter assay. Previously, we have shown that the natural product 1 only recruits βArr2 to the κOR at high doses (EC₅₀=39 μM).⁴⁰ However, in addition to having increased G-protein potencies, many of the C10-substituted derivatives induce βArr2 recruitment more potently than 1 (FIG. 6). Due to limitations in solubility that prevented testing at doses high enough to observe a plateau and calculate an Eₘₐₓ, bias values could not be accurately calculated. Nevertheless, several important trends could be observed. First, in addition to being more potent than 1 in this assay, compounds 2 and 3 appear to be more efficacious than the reference compound U50,488 at inducing βArr2 recruitment. On the other hand, derivative 5, which was the most potent derivative identified and equipotent with U50,488 in the G-protein assay, induces significantly less recruitment of βArr2 than U50488 at concentrations <10 μM. Taken together, compounds 2, 3, and 5 represent a novel class of potent κOR agonists with distinct G-protein and βArr2 signaling properties.

Previous reports by our lab demonstrated that the natural product 1 displays affinity towards a number of non-κOR CNS targets, albeit at considerably lower affinity than it binds to the κOR.⁴⁰ Because 2 and 3 have increased κOR affinity and the highest selectivity for κOR, we sought to determine whether these modifications at the C10 position would also improve selectivity against other CNS off-targets. To do so, 2 and 3 were screened for affinity against a panel of 54 GPCRs, ion channels, and transporters by the Psychoactive Drug Screening Program (PDSP). An initial radioligand binding screen at a single concentration of 10 μM of each analogue was performed to identify potential off-target receptor affinity (Table S3). In instances where 2 or 3 displaced >50% of radioligand from an off-target, multidose concentration-response curves were generated to calculate binding affinities (K) (Table 2). In comparison to the natural product 1, compounds 2, and 3 exhibit binding affinities at an increased number of targets namely the serotonin receptors and adrenergic receptors. However, 2 and 3 possess relatively weak affinity for these off-targets and have >200-fold greater affinity for the κOR over all off-targets investigated, including the μOR and δOR, indicating they are highly selective κOR ligands.

TABLE 2
Binding affinities (Kᵢ) of 1-3 at CNS receptors and transporters.^{a}

| | 1^{b} | 2 | 3 |
|---|---|---|---|
| Receptor | Kᵢ (nM) | Kᵢ (nM) | Kᵢ (nM) |
| KOR | 166 | 1 | 1 |
| MOR | 3729 | — | — |
| DOR | — | — | — |
| 5-HT2A | 4822 | — | 2314 |
| 5-HT2A | — | — | — |
| 5-HT2B | — | 1794 | 767 |
| 5-HT2B | — | — | — |
| 5-HT3 | 5494 | 210 | 384 |
| 5-HT5A | 2931 | 721 | 829 |
| 5-HT7A | 1408 | 1515 | 1385 |
| Alpha1A | — | 1004 | 883 |
| Alpha1B | — | 2877 | 2017 |
| Alpha1D | — | — | 1140 |
| Alpha2A | — | >10,000 | 732 |
| Alpha2B | 1214 | 1249 | — |
| Alpha2C | 4794 | 2909 | 2864 |
| Alpha7 | — | 6999 | 5488 |
| HERG | — | 1342 | 1102 |
| NET | — | — | 3436 |
| Sigma 2 | — | 2625 | 2218 |

^{a}Binding affinities were determined through the Psychoactive Drug Screening Program (PDSP) utilizing radioligand displacement assays through an average of three independent trials.
“—” indicates compound produced less than 50% displacement of radioligand in primary binding screen at 10 μM.
^{b}Data from reference 40.

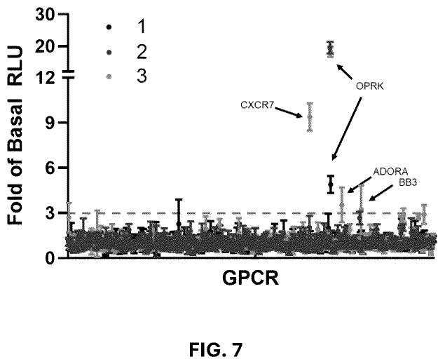

To further probe their selectivity, 1-3 were screened for functional activity against the GPCRome using a PRESTO-Tango assay that measures @Arr2 recruitment to a large panel of GPCRs. Remarkably, 1-3 possess nearly complete selectivity for the κOR and caused very little recruitment of βArr against the 320 non-olfactory GPCRs evaluated (FIG. 7). By comparison, 8 induced recruitment of a number of non-opioid targets in this same assay. The atypical chemokine receptor CXCR7 was identified as a potential target of 3; however, upon further inspection, 3 is a weak agonist compared to the CXCR7 agonist VUF11207 and only activates the receptor at concentrations >3 μM. It is interesting that CXCR7 was also identified as putative target for the indole alkaloid conolidine and metabolites of the indole alkaloid mitragynine.^{55,56} Moreover, CXCR7 is expressed in the same brain regions as the opioid receptors and is believed to be responsible for scavenging opioid peptides.⁵⁷ Thus, CXCR7 appears to be a common target for opioid ligands, especially indole alkaloids; however in the case of 3, these effects are not very potent.

Data collected by the PDSP using PRESTO-Tango to measure βArr recruitment at 3 μM. Compounds 1-3 were screened at 320 non-olfactory GPCRs βArr2 for agonism. Data is presented as the fold-change from basal relative luminesce units (RLUs) from an average of three assays. GPCRs that induced >3-fold change are labeled, while a comprehensive list of GPCRs was reported previously.⁵⁸

### Discussion

The seeds of the akuamma tree have been used as an ethnomedicine in its native West Africa for its pain-relieving effects.^{59,60} These analgesic effects have generally been attributed to the μOR-selective indole alkaloids akuammine and pseudo-akuammigine.^{40,61-63} However, the presence of 1, a selective κOR agonist, also suggests activity at this receptor may be contributing to the observed physiological effects of akuamma seeds.^{64,65} Indeed, the utility of the κOR as a safer target for pain relief has been explored due to its ability to produce analgesic effects without the addictive properties seen with μOR agonists.⁸⁻¹⁰ Moreover, because 1 is structurally distinct from previously investigated opioid ligands, it represents a new opportunity to probe the pharmacology of the κOR by developing a new class of κOR agonists. To that end, this study, which represents the first comprehensive SAR investigation of 1, provided several highly potent and selective κOR agonists.

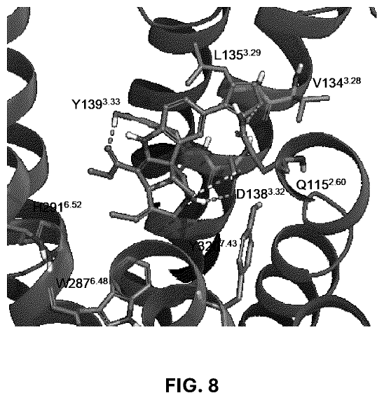

By altering four different sites on the akuammicine scaffold, it became apparent that modifications to the C10 position uniquely led to increased κOR potency. Notably, analogues 2-6, possessing halogens or aromatic substituents, exhibit similar levels of potency as some of the most potent known κOR agonists, including U50,488, EOM salvinorin B, and triazole 1.1. It is noteworthy that the most potent of these compounds, 5, bears a furan ring at C10, which is also found in the potent kappa agonists nalfurafine, triazole 1.1, and salvinorin A.^{16,66,67} Recent cryo-EM studies revealed that the furan rings of nalfurafine and MOM-SalB, a derivative of salvinorin A, fill the same hydrophobic subpocket in the κOR binding site between transmembrane domains TM2 and TM3.^{33,37} Similarly, dichlorobenzene in the arylacetamide GR89,696, and the iodobenzene in the morphan MP1104 also occupy this same space, indicating that this subpocket may be uniquely suited to bind furan rings and aryl halides.^{33,68} Furthermore, the similarity in potencies between 2, 3, and 5 to these other κOR ligands suggests they may be making similar ligand-receptor interactions with this subpocket in order to enhance binding and potency at the κOR. Supporting this hypothesis, in the energy minimized conformation of 5, the furan ring and methyl ester are nearly perfectly aligned with those of MOM-SalB. Moreover, when this superimposed structure of 5 is placed in the κOR binding site, and energy minimized in the context of the receptor-ligand complex, the protonated amine of 5 is positioned in close proximity to Asp138 and forms salt-bridge interactions (FIG. 8). During the course of a 100 nanosecond molecular dynamics simulation with this binding pose as the initial configuration, 5 maintains a similar orientation with persistent salt-bridge formation and the furan ring occupying the TM2-TM3 subpocket.

The structure of 5 was superimposed onto MOM-SalB bound to the κOR (PDB: 8DZP) by aligning their furan rings and esters. The 5-κOR complex was further energy minimized using Prime (Schrödinger Suite 2023-1). Salt-bridge interactions are shown in yellow. Hydrogen bond interactions shown in pink.

Recent studies have suggested that ligand-receptor interactions near the TM2-TM3 subpocket may influence G-protein and βArr2 signaling. In the cryo-EM structure of the κOR bound to nalfurafine, a G-protein-biased agonist, Q115^{2.60} is oriented toward TM3 and can form hydrogen bond interactions with the ligand's furan ring.³⁷ In MD simulations of WMS-X600, a βArr2-biased κOR agonist, and U50,488, a balanced κOR agonist, El Daibani et al. observed Q115^{2.60} frequently is oriented toward TM1.37 In our docking pose and MD simulations, Q115²⁶⁰ is oriented toward the furan ring of 5 (FIG. 8), which is consistent with its reduced potency in the @Arr2 recruitment assay. Conversely, the bromine and iodine atoms of 2 and 3, which more potently and efficaciously promote @Arr2 recruitment, may introduce steric bulk in the subpocket and cause Q115^{2.60} to be oriented toward TM1. Although additional studies are needed to validate this binding pose experimentally, it does appear to be consistent with the SAR trends and signaling properties described in this study.

Regardless of the molecular basis for these effects, the in vitro data clearly demonstrate that the C10-substitution influence κOR potency, selectivity, and signaling properties. Halogenated analogues 2 and 3 display remarkable selectivity for the κOR in both binding and functional activity assays. Examining the effect of 2 and 3 for their ability to induce @Arr2 recruitment unmasked that these analogues recruit @Arr2 with similar potencies and greater efficacies than the reference ligand U50,488. This suggest that 2 and 3 are either balanced or @Arr2 biased agonists at the κOR. Driven in part by early observations that the sedation and aversion may be @Arr2-dependent, most recent κOR ligand development has focused on identifying G-protein biased agonists.^{12,32,37,52,69} By comparison, few balanced or @Arr2 biased κOR agonists have been discovered or investigated. However, not all G-protein biased κOR agonists are devoid of side effects. For example, RB64 produces significant conditioned place aversion.³² Instead, more nuanced events in the signaling cascade may be responsible for these adverse effects.^{70,71} In this light, 5 may prove to be a useful comparison to 2 and 3 since it induces considerably less @Arr2 recruitment than U50,488, 2, and 3 in this assay despite being the most potent derivative in the cAMP assay. Given their potency and selectivity, 2, 3, 5 and other derivatives of 1 will be important tools to explore the behavioral effects of balanced κOR agonist and further refine the importance of @Arr2-dependent signaling in κOR-mediated adverse effects.

Because the major adverse effects caused by κOR activation are centrally mediated, the development of peripherally restricted compounds has also been explored in pursuit of safer κOR agonists.⁷² However, these efforts have generally been limited to peptides, which inherently can suffer from poor oral bioavailability and plasma stability.⁷³ With this in mind, we explored whether peripherally restricted agonists derived from 1 could be developed by alkylating its tertiary nitrogen. Because of the positive charge, these quaternary nitrogen cations are unlikely to transverse the blood-brain barrier. When applied to 1, these modifications led to near complete loss in opioid activity (16-20). However, the bromination of C10 (23-25) was able to rescue much of this activity and resulted in compounds with moderate potency. Further exploration of the SAR may thus reveal other modifications that could lead to additional improvements in this activity and high potency, peripherally restricted κOR agonists. Furthermore, the only alkylated derivative of 1 that retained activity (19), and the most potent alkylated derivative of 2 (25), both contain a cyclopropylmethyl appendage. Notably, cycopropylmehtyl groups are also found on the nitrogen of several morphine-derived κOR ligands, including MP1104, BU74, and buprenorphine, with high κOR affinity.⁷⁴ This provides further support to our binding model depicted in FIG. 5 wherein the tertiary amine of 1 and its derivatives form similar interactions with the κOR as the morphinan nitrogen (i.e., salt-bridge interactions with Asp138^{3,32}).^{68,75}

In conclusion, through a systematic investigation of the akuammicine scaffold this study has uncovered a new class of potent κOR ligands. Of note, halogenation and arylation of the C10 led to dramatic improvements in potency, suggesting the aryl ring of 1 is oriented to a subpocket of the κOR. These studies set the stage for future exploration of this complex natural product scaffold to probe how ligand-receptor interactions dictate downstream signaling events. Additionally, derivatives, 2, 3, and 5 potently activate the κOR G-protein pathway and induce differential responses in the βArr2 recruitment assay indicating they may have unique signaling properties. As such, they offer new pharmacological tools to elucidate the mechanisms that govern the behavioral effects of κOR agonists.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

ADDIN ZOTERO_BIBL {“uncited”:[ ], “omitted”:[ ], “custom”:[ ]}CSL_BIBLIOGRAPHY (1) Pathan, H.; Williams, J. Basic Opioid Pharmacology: An Update. Br. J. Pain 2012, 6 (1), 11-16.
(2) Valentino, R. J.; Volkow, N. D. Untangling the Complexity of Opioid Receptor Function. Neuropsychopharmacol. 2018, 43 (13), 2514-2520.
(3) James, A.; Williams, J. Basic Opioid Pharmacology—An Update. Br. J. Pain 2020, 14 (2), 115-121.
(4) Pasternak, G. W.; Pan, Y.-X. Mu Opioids and Their Receptors: Evolution of a Concept. Pharmacol. Rev. 2013, 65 (4), 1257-1317.
(5) Presley, C. C.; Lindsley, C. W. DARK Classics in Chemical Neuroscience: Opium, a Historical Perspective. ACS Chem. Neurosci. 2018, 9 (10), 2503-2518.
(6) Darcq, E.; Kieffer, B. L. Opioid Receptors: Drivers to Addiction? Nat. Rev. Neurosci. 2018, 19 (8), 499-514.
(7) Pergolizzi, J. V.; Varrassi, G.; Paladini, A.; LeQuang, J. Stopping or Decreasing Opioid Therapy in Patients on Chronic Opioid Therapy. Pain Ther. 2019, 8 (2), 163-176.
(8) Butelman, E. R.; Yuferov, V.; Kreek, M. J. κ-Opioid Receptor/Dynorphin System: Genetic and Pharmacotherapeutic Implications for Addiction. Trends Neurosci. 2012, 35 (10), 587-596.
(9) Knoll, A. T.; Carlezon, W. A. Dynorphin, Stress, and Depression. Brain Res. 2010, 1314, 56-73.
(10) Millan, M. J. κ-Opioid Receptors and Analgesia. Trends Pharmacol. Sci. 1990, 11 (2), 70-76.
(11) Dalefield, M. L.; Scouller, B.; Bibi, R.; Kivell, B. M. The Kappa Opioid Receptor: A Promising Therapeutic Target for Multiple Pathologies. Front. Pharmacol. 2022, 13, 837671.
(12) Dunn, A. D.; Reed, B.; Erazo, J.; Ben-Ezra, A.; Kreek, M. J. Signaling Properties of Structurally Diverse Kappa Opioid Receptor Ligands: Toward in Vitro Models of in Vivo Responses. ACS Chem. Neurosci. 2019, 10 (8), 3590-3600.
(13) Wadenberg, M.-L. G. A Review of the Properties of Spiradoline: A Potent and Selective k-Opioid Receptor Agonist. CNS Drug Rev. 2003, 9 (2), 187-198.
(14) Schattauer, S. S.; Kuhar, J. R.; Song, A.; Chavkin, C. Nalfurafine Is a G-Protein Biased Agonist Having Significantly Greater Bias at the Human than Rodent Form of the Kappa Opioid Receptor. Cell. Signal. 2017, 32, 59-65.
(15) Rives, M.-L.; Rossillo, M.; Liu-Chen, L.-Y.; Javitch, J. A. 6′-Guanidinonaltrindole (6′-GNTI) Is a G Protein-Biased κ-Opioid Receptor Agonist That Inhibits Arrestin Recruitment. J. Biol. Chem. 2012, 287 (32), 27050-27054.
(16) Munro, T. A.; Duncan, K. K.; Xu, W.; Wang, Y.; Liu-Chen, L.-Y.; Carlezon, W. A.; Cohen, B. M.; Béguin, C. Standard Protecting Groups Create Potent and Selective κ Opioids: Salvinorin B Alkoxymethyl Ethers. Bioorg. Med. Chem. 2008, 16 (3), 1279-1286.
(17) Frankowski, K. J.; Hedrick, M. P.; Gosalia, P.; Li, K.; Shi, S.; Whipple, D.; Ghosh, P.; Prisinzano, T. E.; Schoenen, F. J.; Su, Y.; Vasile, S.; Sergienko, E.; Gray, W.; Hariharan, S.; Milan, L.; Heynen-Genel, S.; Mangravita-Novo, A.; Vicchiarelli, M.; Smith, L. H.; Streicher, J. M.; Caron, M. G.; Barak, L. S.; Bohn, L. M.; Chung, T. D. Y.; Aube, J. Discovery of Small Molecule Kappa Opioid Receptor Agonist and Antagonist Chemotypes through a HTS and Hit Refinement Strategy. ACS Chem. Neurosci. 2012, 3 (3), 221-236.
(18) Fugal, J.; Serpa, S. M. Difelikefalin: A New κ-Opioid Receptor Agonist for the Treatment of Hemodialysis-Dependent Chronic Kidney Disease-Associated Pruritus. Ann. Pharmacother. 2023, 57(4), 480-488.
(19) Pfeiffer, A.; Brantl, V.; Herz, A.; Emrich, H. M. Psychotomimesis Mediated by κ Opiate Receptors. Science 1986, 233 (4765), 774-776.
(20) Ranganathan, M.; Schnakenberg, A.; Skosnik, P. D.; Cohen, B. M.; Pittman, B.; Sewell, R. A.; D'Souza, D. C. Dose-Related Behavioral, Subjective, Endocrine, and Psychophysiological Effects of the κ Opioid Agonist Salvinorin A in Humans. Biol. Psychiatry 2012, 72 (10), 871-879.
(21) Tejeda, H. A.; Counotte, D. S.; Oh, E.; Ramamoorthy, S.; Schultz-Kuszak, K. N.; Backman, C. M.; Chefer, V.; O'Donnell, P.; Shippenberg, T. S. Prefrontal Cortical Kappa-Opioid Receptor Modulation of Local Neurotransmission and Conditioned Place Aversion. Neuropsychopharmacol. 2013, 38 (9), 1770-1779.
(22) Fishbane, S.; Mathur, V.; Germain, M. J.; Shirazian, S.; Bhaduri, S.; Munera, C.; Spencer, R. H.; Menzaghi, F.; Aaronson, M.; Alford, K.; Awad, A.; Bhat, P.; Broumand, V.; Calhoun, W.; Darwish, R.; Dua, S.; Dukes, C.; Erinle, A.; Hadley, A.; Hsieh, J.; Kashif, M.; Kopyt, N.; Kumar, J.; Kusnir, J.; Lee, J.; Maasarani, E.; Miller, R.; Mizani, M. R.; Navarro, J.; Podoll, A.; Pohlman, T.; Rivers, D.; Robinson, D.; Rich, L.; Shirazian, S.; Silva, A.; Smith, M.; Topf, J.; Tumlin, J.; Ungar, S.; Zeig, S. Randomized Controlled Trial of Difelikefalin for Chronic Pruritus in Hemodialysis Patients. Kidney Int. Rep. 2020, 5 (5), 600-610.
(23) Zhang, P.; Xiang, S.; Liu, B.; Wang, X.; Yang, X.; Ye, C.; Wang, Z.; Li, Y.; Zhou, L.; Wang, C.; Li, H.; Huang, J.; Peng, A.; Wang, X.; Wang, D.; Xiao, J.; Chen, W.; Cheng, H.; Mao, N.; Wang, J.; Yang, L.; Chen, J. Randomized Controlled Trial of Nalfurafine for Refractory Pruritus in Hemodialysis Patients. Ren. Fail. 45 (1), 2175590.
(24) Morgenweck, J.; Frankowski, K. J.; Prisinzano, T. E.; Aube, J.; Bohn, L. M. Investigation of the Role of B-arrestin-2 in Kappa Opioid Receptor Modulation in a Mouse Model of Pruritus. Neuropharmacol. 2015, 99, 600-609.
(25) Land, B. B.; Bruchas, M. R.; Schattauer, S.; Giardino, W. J.; Aita, M.; Messinger, D.; Hnasko, T. S.; Palmiter, R. D.; Chavkin, C. Activation of the Kappa Opioid Receptor in the Dorsal Raphe Nucleus Mediates the Aversive Effects of Stress and Reinstates Drug Seeking. Proc. Natl. Acad. Sci. 2009, 106 (45), 19168-19173.
(26) Ehrich, J. M.; Messinger, D. I.; Knakal, C. R.; Kuhar, J. R.; Schattauer, S. S.; Bruchas, M. R.; Zweifel, L. S.; Kieffer, B. L.; Phillips, P. E. M.; Chavkin, C. Kappa Opioid Receptor-Induced Aversion Requires P38 MAPK Activation in VTA Dopamine Neurons. J. Neurosci. 2015, 35 (37), 12917-12931.
(27) Bruchas, M. R.; Chavkin, C. Kinase Cascades and Ligand-Directed Signaling at the Kappa Opioid Receptor. Psychopharmacol. (Berl.) 2010, 210 (2), 137-147.
(28) Bruchas, M. R.; Schindler, A. G.; Shankar, H.; Messinger, D. I.; Miyatake, M.; Land, B. B.; Lemos, J. C.; Hagan, C. E.; Neumaier, J. F.; Quintana, A.; Palmiter, R. D.; Chavkin, C. Selective P38a MAPK Deletion in Serotonergic Neurons Produces Stress Resilience in Models of Depression and Addiction. Neuron 2011, 71 (3), 498-511.
(29) Bruchas, M. R.; Land, B. B.; Aita, M.; Xu, M.; Barot, S. K.; Li, S.; Chavkin, C. Stress-Induced P38 Mitogen-Activated Protein Kinase Activation Mediates κ-Opioid-Dependent Dysphoria. J. Neurosci. 2007, 27(43), 11614-11623.
(30) Mores, K. L.; Cummins, B. R.; Cassell, R. J.; van Rijn, R. M. A Review of the Therapeutic Potential of Recently Developed G Protein-Biased Kappa Agonists. Front. Pharmacol. 2019, 10, 407.
(31) French, A. R.; van Rijn, R. M. An Updated Assessment of the Translational Promise of G-Protein-Biased Kappa Opioid Receptor Agonists to Treat Pain and Other Indications without Debilitating Adverse Effects. Pharmacol. Res. 2022, 177, 106091.
(32) White, K. L.; Robinson, J. E.; Zhu, H.; DiBerto, J. F.; Polepally, P. R.; Zjawiony, J. K.; Nichols, D. E.; Malanga, C. J.; Roth, B. L. The G Protein-Biased κ-Opioid Receptor Agonist RB-64 Is Analgesic with a Unique Spectrum of Activities In Vivo. J. Pharmacol. Exp. Ther. 2015, 352 (1), 98-109.
(33) Han, J.; Zhang, J.; Nazarova, A. L.; Bernhard, S. M.; Krumm, B. E.; Zhao, L.; Lam, J. H.; Rangari, V. A.; Majumdar, S.; Nichols, D. E.; Katritch, V.; Yuan, P.; Fay, J. F.; Che, T. Ligand and G-Protein Selectivity in the κ-Opioid Receptor. Nature 2023, 617(7960), 417-425.
(34) Muratspahid, E.; Deibler, K.; Han, J.; Tomašević, N.; Jadhav, K. B.; Olivé-Marti, A.-L.; Hochrainer, N.; Hellinger, R.; Koehbach, J.; Fay, J. F.; Rahman, M. H.; Hegazy, L.; Craven, T. W.; Varga, B. R.; Bhardwaj, G.; Appourchaux, K.; Majumdar, S.; Muttenthaler, M.; Hosseinzadeh, P.; Craik, D. J.; Spetea, M.; Che, T.; Baker, D.; Gruber, C. W. Design and Structural Validation of Peptide-Drug Conjugate Ligands of the Kappa-Opioid Receptor. Nat. Commun. 2023, 14 (1), 8064.
(35) Wang, Y.; Zhuang, Y.; DiBerto, J. F.; Zhou, X. E.; Schmitz, G. P.; Yuan, Q.; Jain, M. K.; Liu, W.; Melcher, K.; Jiang, Y.; Roth, B. L.; Xu, H. E. Structures of the Entire Human Opioid Receptor Family. Cell 2023, 186 (2), 413-427.
(36) Chen, Y.; Chen, B.; Wu, T.; Zhou, F.; Xu, F. Cryo-EM Structure of Human κ-Opioid Receptor-Gi Complex Bound to an Endogenous Agonist Dynorphin A. Protein & Cell 2022, 14(6), 464-468.
(37) El Daibani, A.; Paggi, J. M.; Kim, K.; Laloudakis, Y. D.; Popov, P.; Bernhard, S. M.; Krumm, B. E.; Olsen, R. H. J.; Diberto, J.; Carroll, F. I.; Katritch, V.; Wunsch, B.; Dror, R. O.; Che, T. Molecular Mechanism of Biased Signaling at the Kappa Opioid Receptor. Nat. Commun. 2023, 14 (1), 1338.
(38) Chakraborty, S.; Majumdar, S. Natural Products for the Treatment of Pain: Chemistry and Pharmacology of Salvinorin A, Mitragynine, and Collybolide. Biochemistry 2021, 60 (18), 1381-1400.
(39) Turnaturi, R.; Piana, S.; Spoto, S.; Costanzo, G.; Reina, L.; Pasquinucci, L.; Parenti, C. From Plant to Chemistry: Sources of Active Opioid Antinociceptive Principles for Medicinal Chemistry and Drug Design. Molecules 2023, 28 (20), 7089.
(40) Creed, S. M.; Gutridge, A. M.; Argade, M. D.; Hennessy, M. R.; Friesen, J. B.; Pauli, G. F.; Van Rijn, R. M.; Riley, A. P. Isolation and Pharmacological Characterization of Six Opioidergic Picralima nitida Alkaloids. J. Nat. Prod. 2021, 84 (1), 71-80.
(41) Hennessy, M. R.; Gutridge, A. M.; French, A. R.; Rhoda, E. S.; Meqbil, Y. J.; Gill, M.; Kashyap, Y.; Appourchaux, K.; Paul, B.; Wang, Z. J.; van Rijn, R. M.; Riley, A. P. Modified Akuamma Alkaloids with Increased Potency at the Mu-Opioid Receptor. J. Med. Chem. 2023, 66 (5), 3312-3326.
(42) Wang, J.; Li, R.; Dong, Z.; Liu, P.; Dong, G. Complementary Site-Selectivity in Arene Functionalization Enabled by Overcoming the Ortho Constraint in Palladium/Norbornene Catalysis. Nat. Chem. 2018, 10 (8), 866-872.
(43) Vdradi, A.; Marrone, G. F.; Palmer, T. C.; Narayan, A.; Szabó, M. R.; Le Rouzic, V.; Grinnell, S. G.; Subrath, J. J.; Warner, E.; Kalra, S.; Hunkele, A.; Pagirsky, J.; Eans, S. O.; Medina, J. M.; Xu, J.; Pan, Y.-X.; Borics, A.; Pasternak, G. W.; McLaughlin, J. P.; Majumdar, S. Mitragynine/Corynantheidine Pseudoindoxyls As Opioid Analgesics with Mu Agonism and Delta Antagonism, Which Do Not Recruit β-Arrestin-2. J. Med. Chem. 2016, 59 (18), 8381-8397.
(44) Zhu, S.-F.; Yu, Y.-B.; Li, S.; Wang, L.-X.; Zhou, Q.-L. Enantioselective Hydrogenation of α-Substituted Acrylic Acids Catalyzed by Iridium Complexes with Chiral Spiro Aminophosphine Ligands. Angew. Chem. Int. Ed. 2012, 51 (35), 8872-8875.
(45) Teijaro, C. N.; Munagala, S.; Zhao, S.; Sirasani, G.; Kokkonda, P.; Malofeeva, E. V.; Hopper-Borge, E.; Andrade, R. B. Synthesis and Biological Evaluation of Pentacyclic Strychnos Alkaloids as Selective Modulators of the ABCC10 (MRP7) Efflux Pump. J. Med. Chem. 2014, 57 (24), 10383-10390.
(46) Kokkonda, P.; Brown, K. R.; Seguin, T. J.; Wheeler, S. E.; Vaddypally, S.; Zdilla, M. J.; Andrade, R. B. Biomimetic Total Syntheses of (−)-Leucoridines A and C through the Dimerization of (−)-Dihydrovalparicine. Angew. Chem. Int. Ed. 2015, 54 (43), 12632-12635.
(47) Munagala, S.; Sirasani, G.; Kokkonda, P.; Phadke, M.; Krynetskaia, N.; Lu, P.; Sharom, F. J.; Chaudhury, S.; Abdulhameed, M. D. M.; Tawa, G.; Wallqvist, A.; Martinez, R.; Childers, W.; Abou-Gharbia, M.; Krynetskiy, E.; Andrade, R. B. Synthesis and Evaluation of Strychnos Alkaloids as MDR Reversal Agents for Cancer Cell Eradication. Bioorg. Med. Chem. 2014, 22 (3), 1148-1155.
(48) Olson, K. M.; Duron, D. I.; Womer, D.; Fell, R.; Streicher, J. M. Comprehensive Molecular Pharmacology Screening Reveals Potential New Receptor Interactions for Clinically Relevant Opioids. PLOS ONE 2019, 14 (6), e0217371.
(49) Peng, X.; Knapp, B. I.; Bidlack, J. M.; Neumeyer, J. L. Pharmacological Properties of Bivalent Ligands Containing Butorphan Linked to Nalbuphine, Naltrexone, and Naloxone at μ, δ, and κ Opioid Receptors. J. Med. Chem. 2007, 50 (9), 2254-2258.
(50) Martin-Flores, M.; Singh, B.; Walsh, C. A.; Brooks, E. P.; Taylor, L. C.; Mitchell, L. M. Effects of Buprenorphine, Methylnaltrexone, and Their Combination on Gastrointestinal Transit in Healthy New Zealand White Rabbits. J. Am. Assoc. Lab. Anim. Sci. JAALAS 2017, 56 (2), 155-159.
(51) Bluhm, L. H.; Li, T. Chromatographic Purification of Quaternary Ammonium and Pyridinium Compounds on Normal Phase Silica Gel. Tetrahedron Lett. 1998, 39 (22), 3623-3626.
(52) Brust, T. F.; Morgenweck, J.; Kim, S. A.; Rose, J. H.; Locke, J. L.; Schmid, C. L.; Zhou, L.; Stahl, E. L.; Cameron, M. D.; Scarry, S. M.; Aube, J.; Jones, S. R.; Martin, T. J.; Bohn, L. M. Biased Agonists of the Kappa Opioid Receptor Suppress Pain and Itch without Causing Sedation or Dysphoria. Sci. Signal. 2016, 9 (456), ra117.
(53) Dunn, A. D.; Reed, B.; Guariglia, C.; Dunn, A. M.; Hillman, J. M.; Kreek, M. J. Structurally Related Kappa Opioid Receptor Agonists with Substantial Differential Signaling Bias: Neuroendocrine and Behavioral Effects in C57BL6 Mice. Int. J. Neuropsychopharmacol. 2018, 21 (9), 847-857.
(54) Smith, J. S.; Lefkowitz, R. J.; Rajagopal, S. Biased Signalling: From Simple Switches to Allosteric Microprocessors. Nat. Rev. Drug Discov. 2018, 17(4), 243-260.
(55) Szpakowska, M.; Decker, A. M.; Meyrath, M.; Palmer, C. B.; Blough, B. E.; Namjoshi, O. A.; Chevigne, A. The Natural Analgesic Conolidine Targets the Newly Identified Opioid Scavenger ACKR3/CXCR7. Signal Transduct. Target. Ther. 2021, 6 (1), 1-3.
(56) Chakraborty, S.; Uprety, R.; Slocum, S. T.; Irie, T.; Le Rouzic, V.; Li, X.; Wilson, L. L.; Scouller, B.; Alder, A. F.; Kruegel, A. C.; Ansonoff, M.; Varadi, A.; Eans, S. O.; Hunkele, A.; Allaoa, A.; Kalra, S.; Xu, J.; Pan, Y. X.; Pintar, J.; Kivell, B. M.; Pasternak, G. W.; Cameron, M. D.; McLaughlin, J. P.; Sames, D.; Majumdar, S. Oxidative Metabolism as a Modulator of Kratom's Biological Actions. J. Med. Chem. 2021, 64 (22), 16553-16572.
(57) Meyrath, M.; Szpakowska, M.; Zeiner, J.; Massotte, L.; Merz, M. P.; Benkel, T.; Simon, K.; Ohnmacht, J.; Turner, J. D.; Krüger, R.; Seutin, V.; Ollert, M.; Kostenis, E.; Chevigné, A. The Atypical Chemokine Receptor ACKR3/CXCR7 Is a Broad-Spectrum Scavenger for Opioid Peptides. Nat. Commun. 2020, 11 (1), 3033.
(58) Kroeze, W. K.; Sassano, M. F.; Huang, X.-P.; Lansu, K.; McCorvy, J. D.; Giguere, P. M.; Sciaky, N.; Roth, B. L. PRESTO-Tango as an Open-Source Resource for Interrogation of the Druggable Human GPCRome. Nat. Struct. Mol. Biol. 2015, 22 (5), 362-369.
(59) Erharuyi, O.; Falodun, A.; Langer, P. Medicinal Uses, Phytochemistry and Pharmacology of Picralima nitida (Apocynaceae) in Tropical Diseases: A Review. Asian Pac. J. Trop. Med. 2014, 7 (1), 1-8.
(60) Dapaah, G.; Koffuor, G. A.; Mante, P. K.; Ben, I. O. Antitussive, Expectorant and Analgesic Effects of the Ethanol Seed Extract of Picralima nitida (Stapf) Th. & H. Durand. Res. Pharm. Sci. 2016, 11 (2), 100-112.
(61) Duwiejua, M.; Woode, E.; Obiri, D. D. Pseudo-Akuammigine, an Alkaloid from Picralima nitida Seeds, Has Anti-Inflammatory and Analgesic Actions in Rats. J. Ethnopharmacol. 2002, 81 (1), 73-79.
(62) Woode, E.; Obiri, D.; Ansah, C.; Duwiejua, M. Total Alkaloidal Extract of Picralima nitida (Fam. Apocynaceae) Seeds Has Anti-Inflammatory Actions. J. Ghana Sci. Assoc. 2006, 8 (1), 70-78.
(63) Lewin, G.; Le Ménez, P.; Rolland, Y.; Renouard, A.; Giesen-Crouse, E. Akuammine and Dihydroakuammine, Two Indolomonoterpene Alkaloids Displaying Affinity for Opioid Receptors. J. Nat. Prod. 1992, 55 (3), 380-384.
(64) Levy, J.; Le Men, J.; Janot, M. M. Akuammicine, an Alkaloid from the Seeds of Picralima nitida. Bull. Soc. Chim. Fr. 1960, 979-981.
(65) Menzies, J. R. W.; Paterson, S. J.; Duwiejua, M.; Corbett, A. D. Opioid Activity of Alkaloids Extracted from Picralima nitida (Fam. Apocynaceae). Eur. J. Pharmacol. 1998, 350 (1), 101-108.
(66) Zhou, L.; Lovell, K. M.; Frankowski, K. J.; Slauson, S. R.; Phillips, A. M.; Streicher, J. M.; Stahl, E.; Schmid, C. L.; Hodder, P.; Madoux, F.; Cameron, M. D.; Prisinzano, T. E.; Aube, J.; Bohn, L. M. Development of Functionally Selective, Small Molecule Agonists at Kappa Opioid Receptors. J. Biol. Chem. 2013, 288 (51), 36703-36716.
(67) Von Voigtlander, P. F.; Lewis, R. A. U-50,488, a Selective Kappa Opioid Agonist: Comparison to Other Reputed Kappa Agonists. Prog. Neuropsychopharmacol. Biol. Psychiatry 1982, 6 (4), 467-470.
(68) Mafi, A.; Kim, S.-K.; Goddard, W. A. The Atomistic Level Structure for the Activated Human κ-Opioid Receptor Bound to the Full Gi Protein and the MP1104 Agonist. Proc. Natl. Acad. Sci. U.S.A. 2020, 117 (11), 5836-5843.
(69) Reed, B.; Butelman, E. R.; Kreek, M. J. Kappa Opioid Receptor Antagonists as Potential Therapeutics for Mood and Substance Use Disorders. In The Kappa Opioid Receptor; Liu-Chen, L.-Y., Inan, S., Eds.; Springer International Publishing: Cham, 2022, 473-491.
(70) Liu, J. J.; Chiu, Y.-T.; DiMattio, K. M.; Chen, C.; Huang, P.; Gentile, T. A.; Muschamp, J. W.; Cowan, A.; Mann, M.; Liu-Chen, L.-Y. Phosphoproteomic Approach for Agonist-Specific Signaling in Mouse Brains: mTOR Pathway Is Involved in κ Opioid Aversion. Neuropsychopharmacol. 2019, 44 (5), 939-949.
(71) Liu-Chen, L.-Y.; Huang, P. Signaling Underlying Kappa Opioid Receptor-Mediated Behaviors in Rodents. Front. Neurosci. 2022, 16, 964724.
(72) Beck, T. C.; Dix, T. A. Targeting Peripheral κ-Opioid Receptors for the Non-Addictive Treatment of Pain. Future Drug Discov. 2019, 1 (2), FDD17.
(73) Martinez, V.; Abalo, R. Peripherally Acting Opioid Analgesics and Peripherally-Induced Analgesia. Behav. Pharmacol. 2020, 31 (2 & 3), 136-158.
(74) Husbands, S. M.; Neilan, C. L.; Broadbear, J.; Grundt, P.; Breeden, S.; Aceto, M. D.; Woods, J. H.; Lewis, J. W.; R. Traynor, J. BU74, a Complex Oripavine Derivative with Potent Kappa Opioid Receptor Agonism and Delayed Opioid Antagonism. Eur. J. Pharmacol. 2005, 509 (2), 117-125.
(75) Huang, W.; Manglik, A.; Venkatakrishnan, A. J.; Laeremans, T.; Feinberg, E. N.; Sanborn, A. L.; Kato, H. E.; Livingston, K. E.; Thorsen, T. S.; Kling, R.; Granier, S.; Gmeiner, P.; Husbands, S. M.; Traynor, J. R.; Weis, W. I.; Steyaert, J.; Dror, R. O.; Kobilka, B. K. Structural Insights into μ-Opioid Receptor Activation. Nature 2015, 524 (7565), 315-321.
(76) Hong, A. Y.; Vanderwal, C. D. A Synthesis of Alsmaphorazine B Demonstrates the Chemical Feasibility of a New Biogenetic Hypothesis. J. Am. Chem. Soc. 2015, 137(23), 7306-7309.
(77) Paton, K.; Robichon, K.; Templeton, N.; Denny, L.; Al Abadey, A.; Luo, D.; Prisinzano, T.; La Flamme, A.; & Kivell, B. The Salvinorin Analogue, Ethoxymethyl Ether Salvinorin B, Promotes Remyelination in Preclinical Models of Multiple Sclerosis. Front. Neurol., 2021, 12, 782190.
(78) Cassell, R. J.; Mores, K. L.; Zerfas, B. L.; Mahmoud, A. H.; Lill, M. A.; Trader, D. J.; van Rijn, R. M. Rubiscolins Are Naturally Occurring G Protein-Biased Delta Opioid Receptor Peptides. Eur. Neuropsychopharmacol. 2019, 29 (3), 450-456.
(79) Jo, S.; Kim, T.; Iyer, V. G.; Im, W. CHARMM-GUI: A Web-Based Graphical User Interface for CHARMM. J. Comput. Chem. 2008, 29 (11), 1859-1865.
(80) Phillips, J. C.; Braun, R.; Wang, W.; Gumbart, J.; Tajkhorshid, E.; Villa, E.; Chipot, C.; Skeel, R. D.; Kalé, L.; Schulten, K. Scalable Molecular Dynamics with NAMD. J. Comput. Chem. 2005, 26 (16), 1781-1802.
(81) Huang, J.; Rauscher, S.; Nawrocki, G.; Ran, T.; Feig, M.; de Groot, B. L.; Grubmüller, H.; MacKerell, A. D. CHARMM36m: An Improved Force Field for Folded and Intrinsically Disordered Proteins. Nat. Methods 2017, 14 (1), 71-73.
(82) Vanommeslaeghe, K.; Hatcher, E.; Acharya, C.; Kundu, S.; Zhong, S.; Shim, J.; Darian, E.; Guvench, O.; Lopes, P.; Vorobyov, I.; Mackerell Jr., A. D. CHARMM General Force Field: A Force Field for Drug-like Molecules Compatible with the CHARMM All-Atom Additive Biological Force Fields. J. Comput. Chem. 2010, 31 (4), 671-690.
(83) Chio, H.; Guest, E. E.; Hobman, J. L.; Dottorini, T.; Hirst, J. D.; Stekel, D. J. Predicting Bioactivity of Antibiotic Metabolites by Molecular Docking and Dynamics. J. Mol. Graph. Model. 2023, 123, 108508.
(84) de Oliveira, T. A.; Medaglia, L. R.; Maia, E. H. B.; Assis, L. C.; de Carvalho, P. B.; da Silva, A. M.; Taranto, A. G. Evaluation of Docking Machine Learning and Molecular Dynamics Methodologies for DNA-Ligand Systems. Pharmaceuticals 2022, 15 (2), 132.
(85) Humphrey, W.; Dalke, A.; Schulten, K. VMD: Visual Molecular Dynamics. J. Mol. Graph. 1996, 14 (1), 33-38, 27-28.

1. A compound of formula I 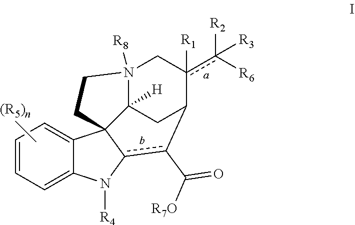
or a pharmaceutically acceptable salt thereof,
wherein
each [symbol] independently represents a single or double bond;
R₁ is absent when [symbol] is a double bond or R₁ is hydrogen, hydroxyl, or acetyloxy when [symbol] is a single bond;
R₂ is hydrogen;
R₃ is absent when [symbol] is a double bond or is hydrogen, hydroxyl, or acetyloxy when [symbol] is a single bond, or R₂ and R₃ together form a carbonyl group when [symbol] is a single bond;
R₄ is hydrogen, substituted or unsubstituted alkyl, or aralkyl;
R₆ and R₇ are alkyl; and
R₅ is selected from hydrogen, hydroxy, alkoxy, hydroxyalkyl, alkoxyalkyl, halide, haloalkyl, nitrile, nitro, amino, amido, acetyl, acetylamino, acetyloxy, substituted or unsubstituted alkyl, substituted or unsubstituted cycloalkyl, substituted or unsubstituted heterocycloalkyl, substituted or unsubstituted aryl, or substituted or unsubstituted heteroaryl, wherein at least one of R₅ is not hydrogen;
n is 1, 2, 3, or 4; and
R₈ is absent or is a substituted or unsubstituted alkyl, an alkenyl group, a cycloalkyl group, or an aralkyl group,
provided the compound of formula I does not include:

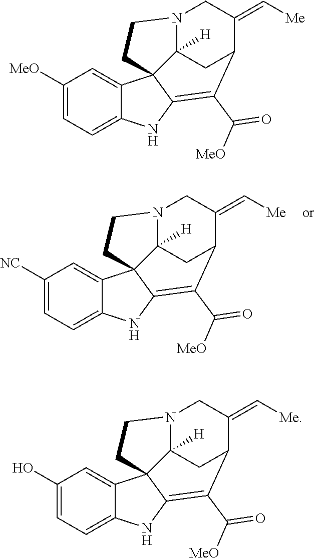

2. The compound of claim 1, wherein n is 1.

3. The compound of claim 1, wherein [symbol] is a double bond and [symbol] is a double bond.

4. The compound of claim 1, wherein R₇ is a C1 to C5 alkyl group.

5. The compound of claim 1, wherein R₄ is hydrogen.

6. The compound of claim 1, wherein R₆ is a C1 to C5 alkyl group.

7. The compound of claim 1, wherein R₈ is absent.

8. The compound of claim 1, wherein the compound is formula II 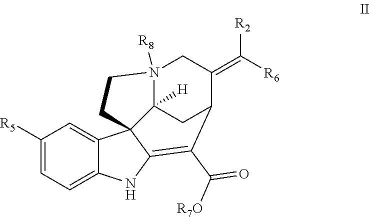
or a pharmaceutically acceptable salt thereof,
wherein
R₂ is hydrogen;
R₆ and R₇ are alkyl; and
R₅ is selected from hydroxy, alkoxy, hydroxyalkyl, alkoxyalkyl, halide, haloalkyl, nitrile, nitro, amino, amido, acetyl, acetylamino, acetyloxy, substituted or unsubstituted alkyl, substituted or unsubstituted cycloalkyl, substituted or unsubstituted heterocycloalkyl, substituted or unsubstituted aryl, or substituted or unsubstituted heteroaryl; and
R₈ is absent or is a substituted or unsubstituted alkyl, an alkenyl group, a cycloalkyl group, or an aralkyl group.

9. The compound of claim 9, wherein R₇ is a C1 to C5 alkyl group.

10. The compound of claim 9, wherein R₈ is absent and R₇ is a methyl group.

11. The compound of claim 9, wherein and R₂ is hydrogen and R₆ is a C1 to C5 alkyl group.

12. The compound of claim 9, wherein and R₂ is hydrogen and R₆ is a methyl group.

13. The compound of claim 9, wherein R₅ is a halide or a substituted or unsubstituted heteroaryl.

14. The compound of claim 1, wherein the compound is formula III 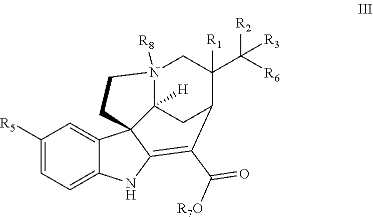
or a pharmaceutically acceptable salt thereof,
wherein
R₁ is hydrogen, hydroxyl, or acetyloxy;
R₂ is hydrogen;
R₃ is hydrogen, hydroxyl, or acetyloxy, or R₂ and R₃ together form a carbonyl group;
R₆ and R₇ are alkyl; and
R₅ is selected from hydrogen, hydroxy, alkoxy, hydroxyalkyl, alkoxyalkyl, halide, haloalkyl, nitrile, nitro, amino, amido, acetyl, acetylamino, acetyloxy, substituted or unsubstituted alkyl, substituted or unsubstituted cycloalkyl, substituted or unsubstituted heterocycloalkyl, substituted or unsubstituted aryl, or substituted or unsubstituted heteroaryl; and
R₈ is absent or is a substituted or unsubstituted alkyl, an alkenyl group, a cycloalkyl group, or an aralkyl group.

15. The compound of claim 1, wherein the compound is formula IV 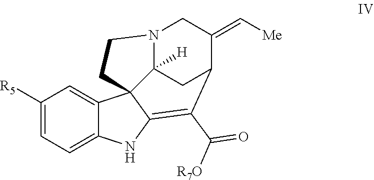
or a pharmaceutically acceptable salt thereof,
wherein
R₇ is alkyl; and
R₅ is selected from halide, substituted or unsubstituted heteroaryl, or substituted or unsubstituted heteroaryl.

16. The compound of claim 1 selected from

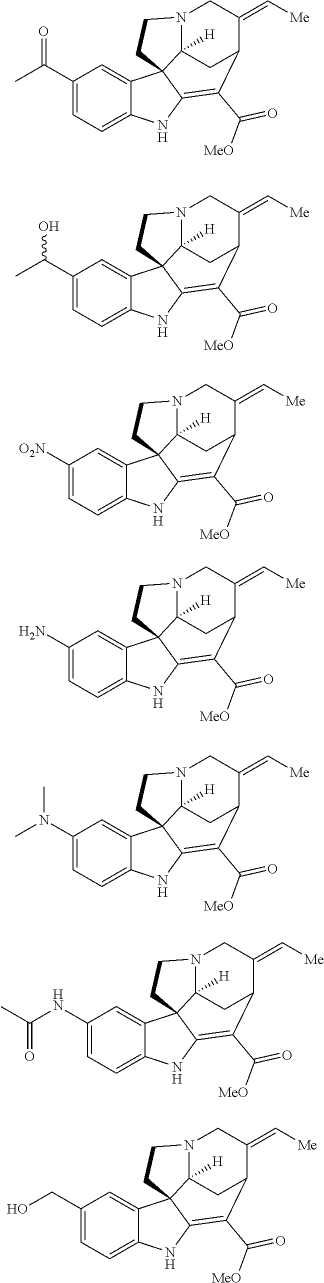

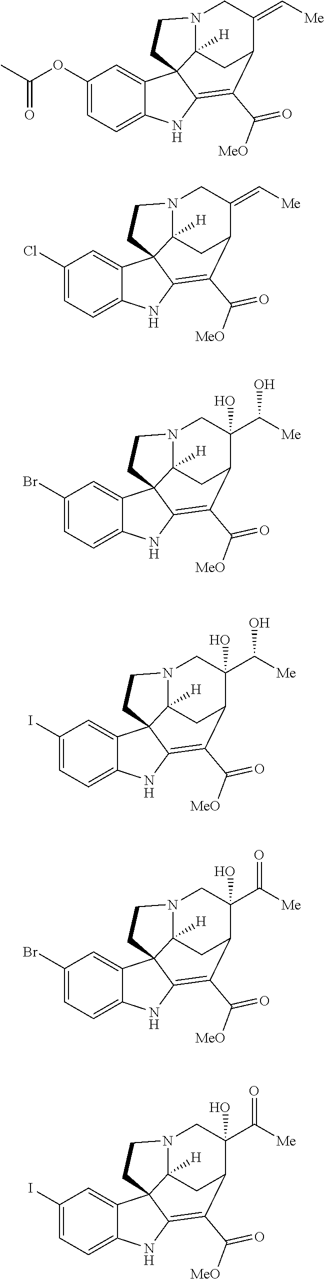

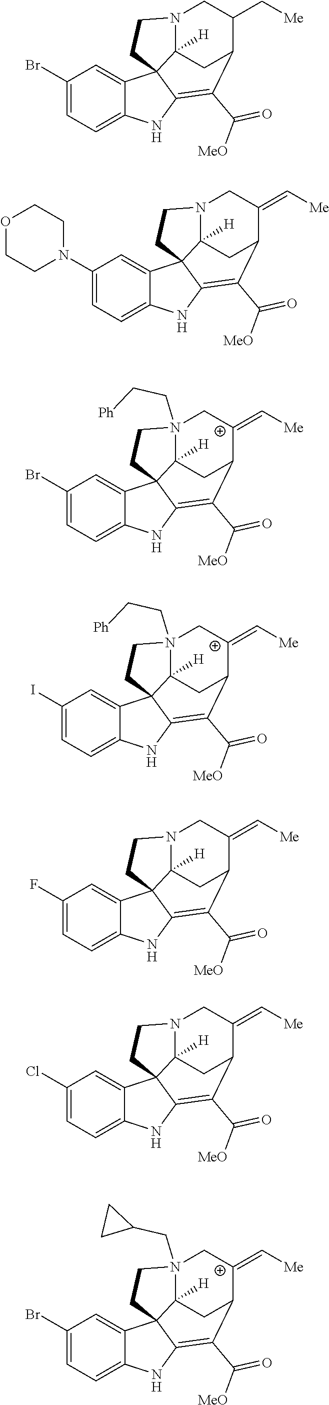

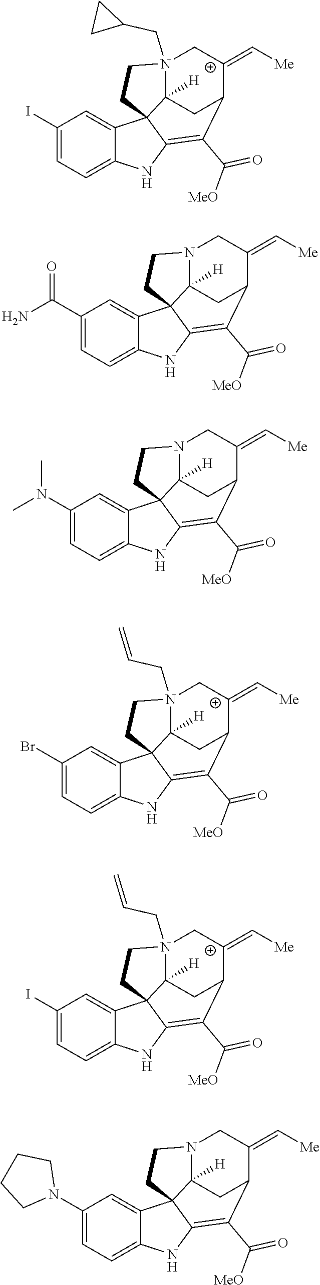

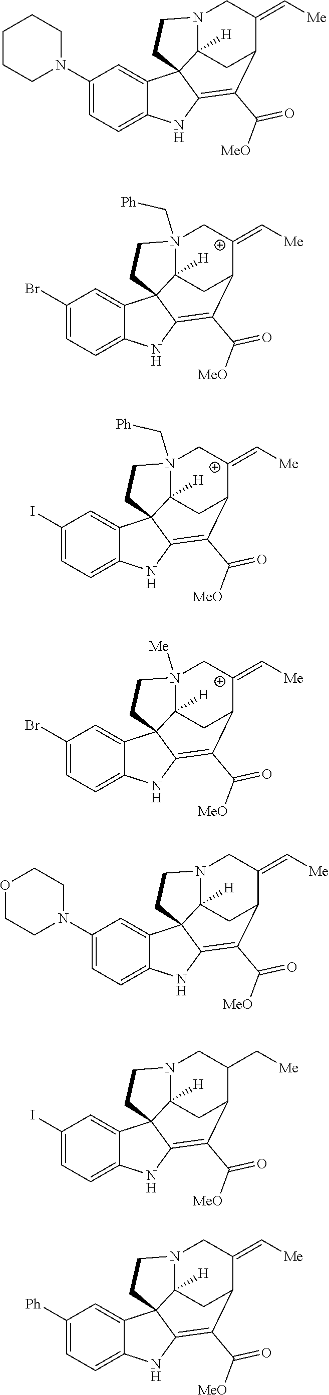

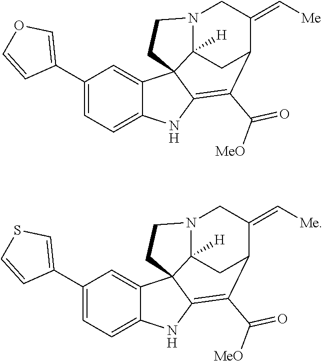

17. The compound of claim 1 selected from

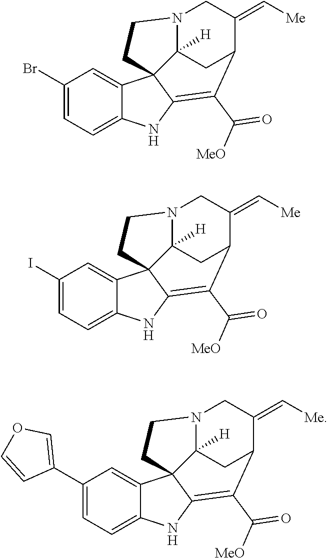

18. A pharmaceutical composition comprising the compound of claim 1 and a pharmaceutically acceptable carrier.

19. A method of treating or preventing pain in a subject comprising administering to the subject a compound of claim 1.

20. The method of claim 19, wherein the subject has chronic pain.
